(19) United States
(12) Patent Application Publication
(10) Pub. No.: US 2022/0331303 A1
(43) Pub. Date: Oct. 20, 2022

(54) USE OF AN OREXIN 2 RECEPTOR AGONIST FOR THE TREATMENT OF EXCESSIVE SLEEPINESS

(71) Applicant: Takeda Pharmaceutical Company Limited, Osaka (JP)

(72) Inventors: Deborah HARTMAN, Cambridge, MA (US); Rebecca EVANS, Cambridge, MA (US); Helene FAESSEL, Cambridge, MA (US); Robert RUBENS, Cambridge, MA (US); Shinichiro TANAKA, Cambridge, MA (US)

(21) Appl. No.: 17/642,337

(22) PCT Filed: Sep. 12, 2020
(86) PCT No.: PCT/IB2020/058482

§ 371 Date: Mar. 11, 2022

Related U.S. Application Data
(60) Provisional application No. 63/031,687, filed on May 29, 2020.
(60) Provisional application No. 62/900,310, filed on Sep. 13, 2019.

(51) Int. Cl. A61K 31/445; A61P 25/26; A61K 45/06

(52) U.S. Cl. CPC A61K 31/445; A61P 25/26; A61K 45/06

(57) ABSTRACT

Described herein are methyl 3-((methylsulfonyl)amino)-2-(((4-phenylcyclohexyl)oxy)methyl)-piperidine-1-carboxylate (Compound (I)), compositions comprising Compound (I), and the use of Compound (I) for the treatment of excessive sleepiness in a subject in need thereof.

### CROSS-REFERENCE TO RELATED APPLICATIONS

This application claims priority to U.S. Provisional Application Nos. 62/900,310 filed Sep. 13, 2019 and 63/031,687 filed May 29, 2020 the entire contents of both of which are incorporated herein by reference in their entireties.

### BACKGROUND OF THE INVENTION

Excessive sleepiness, or excessive daytime sleepiness (EDS), is characterized by persistent sleepiness and often a general lack of energy, even during the day after apparently adequate or even prolonged nighttime sleep. Excessive sleepiness can affect the ability to function in family, social, occupational, or other settings. Current therapies do not adequately address the full extent and spectrum of excessive sleepiness in clinical practice.

The present disclosure satisfies this need and includes a treatment using methyl 3-((methylsulfonyl)amino)-2-(((4-phenylcyclohexyl)oxy)methyl)piperidine-1-carboxylate (Compound (I)), and compositions comprising Compound (I). The present disclosure also includes embodiments wherein Compound (I) is the specific compound methyl (2R,3S)-3-((methylsulfonyl)amino)-2-(((cis-4-phenylcyclohexyl)oxy)methyl)piperidine-1-carboxylate (Compound A).

### SUMMARY OF THE INVENTION

An embodiment of the invention is a method for decreasing or treating excessive sleepiness in a subject in need thereof, comprising administering to the subject an effective amount of Compound (I), or a salt thereof, wherein plasma concentration for Compound (I) is about 50.90 ng/mL or more for about 1 hour or more.

Another embodiment is a method for treating narcolepsy type 2 or idiopathic hypersomnia in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 50.90 ng/mL or more for about 1 hour or more.

Another embodiment is a method for treating shift work disorder, shift work sleep disorder or jet lag syndrome in a subject in need thereof, comprising administering to the subject an effective amount of Compound (I), or a salt thereof, wherein plasma concentration for Compound (I) is about 50.90 ng/mL or more for about 1 hour or more.

Another embodiment is a method for increasing wakefulness in a subject in need thereof, comprising administering to the subject an effective amount of Compound (I), or a salt thereof, wherein plasma concentration for Compound (I) is about 50.90 ng/mL or more for about 1 hour or more.

Another embodiment is a method for increasing sleep latency in maintenance of wakefulness test (MWT) in a subject in need thereof, comprising administering to the subject an effective amount of Compound (I), or a salt thereof, wherein plasma concentration for Compound (I) is about 50.90 ng/mL or more for about 1 hour or more.

Another embodiment is a method for decreasing or improving objective sleepiness or sleepiness measured by EEG in a subject in need thereof, comprising administering to the subject an effective amount of Compound (I), or a salt thereof, wherein plasma concentration for Compound (I) is about 50.90 ng/mL or more for about 1 hour or more.

Another embodiment is a method for improving Karolinska Sleepiness Scale (KSS) rating in a subject in need thereof, comprising administering to the subject an effective amount of Compound (I), or a salt thereof, wherein plasma concentration for Compound (I) is about 50.90 ng/mL or more for about 1 hour or more.

Another embodiment is a method for decreasing or improving subjective sleepiness in a subject in need thereof, comprising administering to the subject an effective amount of Compound (I), or a salt thereof, wherein plasma concentration for Compound (I) is about 50.90 ng/mL or more for about 1 hour or more.

Another embodiment is a method for increasing wakefulness or decreasing excessive sleepiness for about 4 hours or more in a subject in need thereof, comprising administering to the subject an effective amount of Compound (I), or a salt thereof, wherein orexin level in the subject is not compromised or partially compromised; and plasma concentration for Compound (I) is maintained at about 50.90 ng/mL or more.

Another embodiment is a method for improving Epworth Sleepiness Scale (ESS) rating in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 38.21 ng/mL or more for about 1 hour or more.

Another embodiment is a method for treating narcolepsy type 2 in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein the plasma concentration for Compound (I) is about 38.21 ng/mL or more for about 1 hour or more.

Another embodiment is a method for decreasing or treating excessive daytime sleepiness in a subject with obstructive sleep apnea who uses continuous positive airway pressure (CPAP) in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein the plasma concentration for Compound (I) is about 42.08 ng/mL or more for about 1 hour or more.

Another embodiment is a pharmaceutical composition comprising (a) Compound (I), or a salt thereof, and (b) a pharmaceutically acceptable carrier therefor, which provides a plasma concentration for Compound (I) of about 50.90 ng/mL or more for about 1 hour or more.

### BRIEF DESCRIPTION OF THE DRAWINGS

FIG. 1 shows the mean plasma concentration-time profiles of Compound A administered via a single infusion of 9 hours to healthy male subjects. HD: high dose, Compound A 112 mg. LD: low dose, Compound A 44 mg.

FIG. 2 shows LS mean (±SE) of latency to sleep onset versus time. HD: high dose, Compound A 112 mg. LD: low dose, Compound A 44 mg. LS: Least Squares. M: modafinil. P: placebo.

FIG. 3 shows LS Mean (±SE) of Duration of Total Microsleeps (Sec) versus time. HD: high dose, Compound A 112 mg. LD: low dose, Compound A 44 mg. LS: Least Squares. M: modafinil 300 mg. P: placebo.

FIG. 4 shows LS Mean (±SE) of Total Microsleeps versus time. HD: high dose, Compound A 112 mg. LD: low dose, Compound A 44 mg. LS: Least Squares. M: modafinil 300 mg. P: placebo.

FIG. 5 shows LS Mean (±SE) of Total Sleep Time (Min) versus time. HD: high dose, Compound A 112 mg. LD: low dose, Compound A 44 mg. LS: Least Squares. M: modafinil 300 mg. P: placebo.

FIG. 6 shows LS Mean (±SE) of Total Wake Time (Min) versus time. HD: high dose, Compound A 112 mg. LD: low dose, Compound A 44 mg. LS: Least Squares. M: modafinil 300 mg. P: placebo.

FIG. 7 shows a scatterplot of sleep latency versus Compound A plasma concentration. HD: high dose, Compound A 112 mg. LD: low dose, Compound A 44 mg. P: placebo.

FIG. 8 shows a scatter plot of sleep latency versus Compound A plasma concentration by scheduled time.

FIG. 9 shows an overview of the Study Schedule (NT2 Patients).

FIG. 10 shows an overview of Schedule of Study Procedures (NT2 Patients).

FIG. 11A shows Mean and Standard Deviation Plot of Plasma Concentrations of Compound A given as a 9-hour IV infusion on Day 1 in NT2 patients (Cohort C1, C2) (PK Set).

FIG. 11B shows Mean and Standard Deviation Plot of Plasma Concentrations of Compound A given as a 9-hour IV infusion on Day 7 in NT2 patients (Cohort C1, C2) (PK Set).

FIG. 12A shows an average Sleep Latency in MWT in NT2 patients (Cohorts C1, C2).

FIG. 12B shows Change from Baseline by Visit in NT2 patients (Cohorts C1, C2).

FIG. 13 shows Mean and Standard Deviation Plot of Sleep Latency in Each Session in MWT by Visit in NT2 patients (Cohorts C1, C2).

FIG. 14 shows Mean and Standard Deviation Plot of Change from Time-matched Baseline in KSS in NT2 patients (Cohorts C1, C2).

FIG. 15 shows Mean (±SE) Compound A Plasma Concentration-time Profiles for each Compound A Treatment.

FIG. 16A shows a graph of Sleep Latency for Maintenance of Wakefulness Test over time.

FIG. 16B shows a graph of Least Square Mean Differences in Sleep Latency from Placebo over time.

FIG. 17 shows a graph of Least Square Mean Differences in Karolinska Sleepiness Scale from Placebo over time.

### DETAILED DESCRIPTION OF THE INVENTION

The methods, compositions and uses disclosed herein include treating diseases or disorders or symptoms associated with excessive sleepiness in a subject in need thereof, as well as treating subjects suffering from excessive sleepiness who have not been diagnosed with any disease or disorder. Multiple causes have been attributed to excessive sleepiness, which include but are not limited to abnormal sleep quantity or sleep quality, as well as neurological, psychological, cardiac, and pulmonary disorders.

In an embodiment, the methods, compositions and uses of this disclosure may be directed to treating excessive sleepiness caused by reduced levels of orexin, neuropeptide that regulates arousal, wakefulness, and appetite. Excessive sleepiness can also occur in individuals who do not have deficiency of orexin. This disclosure is directed to treating diseases, disorders, and/or symptoms of excessive sleepiness that are not associated with reduced orexin level.

An embodiment of the invention relates to methyl 3-((methylsulfonyl)amino)-2-(((4-phenylcyclohexyl)oxy)methyl)piperidine-1-carboxylate (Compound (I)), or a salt thereof, compositions and kits comprising Compound (I), or a salt thereof, and methods of using Compound (I), or a salt thereof.

Another embodiment is a method for decreasing or treating excessive sleepiness in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 50.90 ng/mL or more for about 1 hour or more.

Further disclosed herein is methyl 3-((methylsulfonyl)amino)-2-(((4-phenyl-cyclohexyl)oxy)methyl)piperidine-1-carboxylate (Compound (I)), or a salt thereof for use in decreasing or treating excessive sleepiness in a subject in need thereof.

Disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for decreasing or treating excessive sleepiness in a subject in need thereof.

Disclosed herein are methods for treating narcolepsy type 2 or idiopathic hypersomnia in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof. In some embodiments, the plasma concentration for Compound (I) is about 50.90 ng/mL or more for about 1 hour or more.

Disclosed herein is methyl 3-((methylsulfonyl)amino)-2-(((4-phenylcyclohexyl)oxy)methyl)piperidine-1-carboxylate (Compound (I)), or a salt thereof for use in treating narcolepsy type 2 or idiopathic hypersomnia in a subject in need thereof. In some embodiments, the plasma concentration for Compound (I) is about 50.90 ng/mL or more for about 1 hour or more.

Disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for treating narcolepsy type 2 or idiopathic hypersomnia in a subject in need thereof. In some embodiments, the plasma concentration for Compound (I) is about 50.90 ng/mL or more for about 1 hour or more.

Disclosed herein are methods for treating shift work disorder, shift work sleep disorder or jet lag syndrome in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 50.90 ng/mL or more for about 1 hour or more.

Further disclosed herein is methyl 3-((methylsulfonyl)amino)-2-(((4-phenyl-cyclohexyl)oxy)methyl)piperidine-1-carboxylate (Compound (I)), or a salt thereof for use in treating shift work disorder, shift work sleep disorder or jet lag syndrome in a subject in need thereof.

Disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for treating shift work disorder, shift work sleep disorder or jet lag syndrome in a subject in need thereof.

Disclosed herein are methods for increasing wakefulness in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 50.90 ng/mL or more for about 1 hour or more.

Further disclosed herein is methyl 3-((methylsulfonyl)amino)-2-(((4-phenyl-cyclohexyl)oxy)methyl)piperidine-1-carboxylate (Compound (I)), or a salt thereof for use in increasing wakefulness in a subject in need thereof.

Further disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for increasing wakefulness in a subject in need thereof.

Disclosed herein are methods for increasing sleep latency in maintenance of wakefulness test (MWT) in a subject in need thereof, comprising administering to the subject an effective amount of methyl 3-((methylsulfonyl)amino)-2-(((4-phenylcyclohexyl)-oxy)methyl)piperidine-1-carboxylate (Compound (I)), or a salt thereof, wherein plasma concentration for Compound (I) is about 50.90 ng/mL or more for about 1 hour or more.

Further disclosed herein is methyl 3-((methylsulfonyl)amino)-2-(((4-phenylcyclohexyl)oxy)methyl)piperidine-1-carboxylate (Compound (I)), or a salt thereof for use in increasing sleep latency in maintenance of wakefulness test (MWT) in a subject in need thereof.

Disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for increasing sleep latency in maintenance of wakefulness test (MWT) in a subject in need thereof.

Disclosed herein are methods for decreasing or improving objective sleepiness or sleepiness measured by electroencephalogram (EEG) in a subject in need thereof, comprising administering to the subject an effective amount of methyl 3-((methylsulfonyl)amino)-2-(((4-phenylcyclohexyl)-oxy)methyl)piperidine-1-carboxylate (Compound (I)), or a salt thereof, wherein plasma concentration for Compound (I) is about 50.90 ng/mL or more for about 1 hour or more.

Further disclosed herein is methyl 3-((methylsulfonyl)amino)-2-(((4-phenyl-cyclohexyl)-oxy)methyl)piperidine-1-carboxylate (Compound (I)), or a salt thereof for use in decreasing or improving objective sleepiness or sleepiness measured by EEG in a subject in need thereof.

Disclosed herein are uses of methyl 3-((methylsulfonyl)amino)-2-(((4-phenyl-cyclohexyl)oxy)methyl)piperidine-1-carboxylate (Compound (I)), or a salt thereof for the manufacture of a medicament for decreasing or improving objective sleepiness or sleepiness measured by electroencephalogram (EEG) in a subject in need thereof.

Disclosed herein are methods for improving Karolinska Sleepiness Scale (KSS) rating in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 50.90 ng/mL or more for about 1 hour or more.

Further disclosed herein is methyl 3-((methylsulfonyl)amino)-2-(((4-phenylcyclohexyl)oxy)methyl)piperidine-1-carboxylate (Compound (I)), or a salt thereof for use in improving Karolinska Sleepiness Scale (KSS) rating in a subject in need thereof.

Disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for improving Karolinska Sleepiness Scale (KSS) rating in a subject in need thereof.

Disclosed herein are methods for decreasing or improving subjective sleepiness in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 50.90 ng/mL or more for about 1 hour or more.

Further disclosed herein is methyl 3-((methylsulfonyl)amino)-2-(((4-phenylcyclohexyl)oxy)methyl)piperidine-1-carboxylate (Compound (I)), or a salt thereof for use in decreasing or improving subjective sleepiness in a subject in need thereof.

Disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for decreasing or improving subjective sleepiness in a subject in need thereof.

Disclosed herein are methods for increasing wakefulness or decreasing excessive sleepiness for about 4 hours or more in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein orexin level in the subject is not compromised or partially compromised; and wherein plasma concentration for Compound (I) is maintained at about 50.90 ng/mL or more.

Disclosed herein is methyl 3-((methylsulfonyl)amino)-2-(((4-phenylcyclohexyl)-oxy)methyl)piperidine-1-carboxylate (Compound (I)), or a salt thereof for use in increasing wakefulness or decreasing excessive sleepiness for about 4 hours or more in a subject in need thereof.

Disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for increasing wakefulness or decreasing excessive sleepiness for about 4 hours or more in a subject in need thereof.

Disclosed herein are methods for treating excessive sleepiness not associated with an orexin deficiency in a subject in need thereof, comprising administering to the subject an effective amount of methyl 3-((methylsulfonyl)amino)-2-(((4-phenylcyclohexyl)-oxy)methyl)piperidine-1-carboxylate (Compound (I)), or a salt thereof, wherein plasma concentration for Compound (I) is about 50.90 ng/mL or more for about 1 hour or more.

Further disclosed herein is methyl 3-((methylsulfonyl)amino)-2-(((4-phenylcyclohexyl)oxy)methyl)piperidine-1-carboxylate (Compound (I)), or a salt thereof for use in treating excessive sleepiness not associated with an orexin deficiency in a subject in need thereof.

Disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for treating excessive sleepiness not associated with an orexin deficiency in a subject in need thereof.

Disclosed herein are methods for improving Epworth Sleepiness Scale (ESS) rating in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 50.90 ng/mL or more for about 1 hour or more. In some embodiments, the plasma concentration for Compound (I) is about 38.21 ng/mL or more for about 1 hour or more. In some embodiments, the subject is suffering from or diagnosed as narcolepsy type 2.

Further disclosed herein is methyl 3-((methylsulfonyl)amino)-2-(((4-phenylcyclohexyl)oxy)methyl)piperidine-1-carboxylate (Compound (I)), or a salt thereof for use in improving Epworth Sleepiness Scale (ESS) rating in a subject in need thereof. In some embodiments, the plasma concentration for Compound (I) is about 50.90 ng/mL or more for about 1 hour or more. In some embodiments, the plasma concentration for Compound (I) is about 38.21 ng/mL or more for about 1 hour or more. In some embodiments, the subject is suffering from or diagnosed as narcolepsy type 2.

Disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for improving Epworth Sleepiness Scale (ESS) rating in a subject in need thereof. In some embodiments, the plasma concentration for Compound (I) is about 50.90 ng/mL or more for about 1 hour or more. In some embodiments, the plasma concentration for Compound (I) is about 38.21 ng/mL or more for about 1 hour or more. In some embodiments, the subject is suffering from or diagnosed as narcolepsy type 2.

Disclosed herein are methods for treating narcolepsy type 2 in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof. In some embodiments, the plasma concentration for Compound (I) is about 50.90 ng/mL or more for about 1 hour or more. In some embodiments, the plasma concentration for Compound (I) is about 38.21 ng/mL or more for about 1 hour or more. In some embodiments, Compound (I) is repeatedly administered for 7 days or more.

Disclosed herein is methyl 3-((methylsulfonyl)amino)-2-(((4-phenylcyclohexyl)oxy)methyl)piperidine-1-carboxylate (Compound (I)), or a salt thereof for use in treating narcolepsy type 2 in a subject in need thereof. In some embodiments, the plasma concentration for Compound (I) is about 50.90 ng/mL or more for about 1 hour or more. In some embodiments, the plasma concentration for Compound (I) is about 38.21 ng/mL or more for about 1 hour or more. In some embodiments, Compound (I) is repeatedly administered for 7 days or more.

Disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for treating narcolepsy type 2 in a subject in need thereof. In some embodiments, the plasma concentration for Compound (I) is about 50.90 ng/mL or more for about 1 hour or more. In some embodiments, the plasma concentration for Compound (I) is about 38.21 ng/mL or more for about 1 hour or more. In some embodiments, Compound (I) is repeatedly administered for 7 days or more.

Disclosed herein are methods for decreasing or treating excessive daytime sleepiness in a subject with obstructive sleep apnea who uses continuous positive airway pressure (CPAP) in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein the plasma concentration for Compound (I) is about 50.90 ng/mL or more for about 1 hour or more. In some embodiments, the plasma concentration for Compound (I) is about 42.08 ng/mL or more for about 1 hour or more. In some embodiments, the plasma concentration for Compound (I) is about 43.98 ng/mL or more for about 1 hour or more.

Disclosed herein is methyl 3-((methylsulfonyl)amino)-2-(((4-phenylcyclohexyl)oxy)methyl)piperidine-1-carboxylate (Compound (I)), or a salt thereof for use in decreasing or treating excessive daytime sleepiness in a subject with obstructive sleep apnea who uses continuous positive airway pressure (CPAP) in need thereof. In some embodiments, the plasma concentration for Compound (I) is about 50.90 ng/mL or more for about 1 hour or more. In some embodiments, the plasma concentration for Compound (I) is about 42.08 ng/mL or more for about 1 hour or more. In some embodiments, the plasma concentration for Compound (I) is about 43.98 ng/mL or more for about 1 hour or more.

Disclosed herein are uses of methyl 3-((methylsulfonyl)amino)-2-(((4-phenylcyclohexyl)oxy)methyl)piperidine-1-carboxylate (Compound (I)), or a salt thereof for the manufacture of a medicament for decreasing or treating excessive daytime sleepiness in a subject with obstructive sleep apnea who uses continuous positive airway pressure (CPAP) in need thereof. In some embodiments, the plasma concentration for Compound (I) is about 50.90 ng/mL or more for about 1 hour or more. In some embodiments, the plasma concentration for Compound (I) is about 42.08 ng/mL or more for about 1 hour or more. In some embodiments, the plasma concentration for Compound (I) is about 43.98 ng/mL or more for about 1 hour or more.

In any of the embodiments disclosed herein, the plasma concentration for Compound (I) is about 38.21 ng/mL or more for about 1 hour or more, and the plasma concentration for Compound (I) is preferably about 50.90 ng/mL or more for about 1 hour or more, and more preferably about 60.54 ng/mL or more for about 1 hour or more. In some embodiments, the plasma concentration for Compound (I) is about 60.54 ng/mL or more for about 4 hours or more. In some embodiments, plasma concentration for Compound (I) is about 150 ng/mL or more for about 4 hours or more. In some embodiments, the plasma concentration for Compound (I) is also about a half of Cmax for administration of Compound (I) or less at about 1 hour prior to sleep time. In some embodiments, the plasma concentration for Compound (I) is further about a quarter of Cmax for administration of Compound (I) or less at about 1 hour prior to sleep time. In some embodiments, the plasma concentration for Compound (I) is also further about a half of 50.90 ng/mL or less at about 1 hour prior to sleep time. In some embodiments, the plasma concentration for Compound (I) is also further about a quarter of 50.90 ng/mL or less at about 1 hour prior to sleep time. In some embodiments, the Cmax for administration of Compound (I) is about 94.66 ng/mL or more. In some embodiments, the AUC∞ for administration of Compound (I) is about 829 ng*h/mL or more. In some embodiments, orexin level in the subject is not compromised or partially compromised. In some embodiments, the subject suffers from the diseases or disorders or symptoms associated with excessive sleepiness. In some embodiments, the subject is sleep-deprived subject, subject with excessive sleepiness, subject with disruptive regular sleep cycle, or subject with a need to decrease sleepiness.

### Methods and Uses

Excessive sleepiness as used herein is also known as excessive daytime sleepiness (EDS) or excessive need for sleep (ENS). The methods and uses disclosed herein may treat diseases or disorders or symptoms associated with excessive sleepiness in a subject in need thereof. In some embodiments, the excessive sleepiness is caused by any one of the following: insufficient quality or quantity of night time sleep; misalignments of the body's circadian pacemaker with the environment (e.g., caused by requirement to remain awake at night for employment such as shift work or personal obligations such as caretaker for sick, young or old family members), such as jet lag, shift work and other circadian rhythm sleep disorders; another underlying sleep disorder, such as narcolepsy (e.g., narcolepsy type 2, probable narcolepsy), sleep apnea (e.g., obstructive sleep apnea, obstructive sleep apnea with use of continuous positive airway pressure), idiopathic hypersomnia, idiopathic excessive sleepiness, and restless legs syndrome; disorders, such as clinical depression or atypical depression; tumors; head trauma; anemia; kidney failure; hypothyroidism; injury to the central nervous system; drug abuse; genetic vitamin deficiency, such as biotin deficiency; and particular classes of prescription and over the counter medication. In some embodiments, the methods and uses herein are used to treat any one of the following: shift work disorder; shift work sleep disorder; and jet lag syndrome. In some embodiments, the methods and uses herein are used to treat any one of the following: narcolepsy type 2, probable narcolepsy, idiopathic hypersomnia, idiopathic excessive sleepiness, hypersomnia, hypersomnolence, sleep apnea syndrome (e.g., obstructive sleep apnea, obstructive sleep apnea with use of continuous positive airway pressure); or disturbance of consciousness such as coma and the like; and narcolepsy syndrome accompanied by narcolepsy-like symptoms; hypersomnolence or hypersomnia syndrome accompanied by daytime hypersomnia (e.g., Parkinson's disease, Guillain-barre syndrome and Kleine Levin syndrome); excessive daytime sleepiness in Parkinson's disease, Prader-Willi Syndrome, depressions (depression, atypical depression, major depressive disorder, treatment resistant depression), ADHD, sleep apnea syndrome (e.g., obstructive sleep apnea, obstructive sleep apnea with use of continuous positive airway pressure) and other disorders of vigilance; residual excessive daytime sleepiness in sleep apnea syndrome (e.g., obstructive sleep apnea, obstructive sleep apnea with use of continuous positive airway pressure); and the like. Narcolepsy (e.g., narcolepsy type 2, probable narcolepsy) may be diagnosed by diagnostic criteria generally used in the field, e.g., The third edition of the International Classification of Sleep Disorders (ICSD-3) and the fifth edition of the Diagnostic and Statistical Manual of Mental Disorders (DSM-5). In some embodiments, the excessive sleepiness is excessive daytime sleepiness or excessive sleepiness during working hours, or excessive sleepiness or reduced quantity of sleep which is caused by requirement to remain awake at night for employment (e.g., shift work) or personal obligations (e.g., caretaker for sick, young or old family members).

In some embodiments, treating excessive sleepiness may comprise reducing or alleviating one or more symptoms of excessive sleepiness. The one or more symptoms of excessive daytime sleepiness may be selected from drowsiness, languor, inertness, fatigue, sluggishness.

In some embodiments, the subject suffers from the diseases or disorders or symptoms associated with excessive sleepiness. In some embodiments, the subject is a sleep-deprived subject, a subject with excessive sleepiness, a subject with disruptive regular sleep cycle, or a subject with a need to decrease sleepiness.

Orexin, or hypocretin, is a neuropeptide that regulates arousal, wakefulness, and appetite. In some embodiments, excessive sleepiness may be associated with orexin deficiency. In some embodiments, the excessive sleepiness is not associated with reduced orexin level. In some embodiments, the orexin level in the subject is not compromised or partially compromised. In some embodiments, the orexin level of the subject in need thereof is that the cerebral spinal fluid (CSF) hypocretin 1 (orexin A) concentration, measured by immunoreactivity, is more than about 200 pg/mL or about 111 to 200 pg/mL or more than about one-third of values obtained in healthy subjects tested using the same assay.

The methods and uses disclosed herein may increase wakefulness and/or decrease excessive sleepiness in a subject in need thereof. In some embodiments, wakefulness and/or decrease of excessive sleepiness is determined by electroencephalogram (EEG) and/or electromyogram (EMG). In some embodiments, wakefulness and/or decrease of sleepiness is determined by using the Maintenance Wakefulness Test (MWT). The MWT may be quantified by EEG. An electroencephalogram (EEG) is a test that detects electrical activity in the brain, for example, by using small, metal discs or electrodes attached to the scalp.

In some embodiments, the method for increasing wakefulness or decreasing excessive sleepiness for at least about 1, 2, 3, 4, 5, 6, 7, 8, 9, 10, 11, or 12 hours or more. In some embodiments, the method for increasing wakefulness or decreasing excessive sleepiness for about 1, 2, 3, 4, 5, 6, 7, 8, 9, 10, 11, or 12 hours or more. In some embodiments, the method for increasing wakefulness or decreasing excessive sleepiness for about 6 hours or more. In some embodiments, the method for increasing wakefulness or decreasing excessive sleepiness for about 8 hours or more.

The methods and uses disclosed herein may increase sleep latency in maintenance of wakefulness test (MWT) in a subject in need thereof. In some embodiments, the sleep latency in MWT increased by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, or 200% or more. In some embodiments, the sleep latency in MWT increased by about 5%10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, or 200% or more. In some embodiments, the sleep latency in MWT increased by at least about 1, 2, 3, 4, 5, 6, 7, 8, 9, 10, 11, 12, 13, 14, 15, 16, 17, 18, 19, or 20 or more minutes. In some embodiments, the sleep latency in MWT increased by about 1, 2, 3, 4, 5, 6, 7, 8, 9, 10, 11, 12, 13, 14, 15, 16, 17, 18, 19, or 20 or more minutes.

The methods and uses disclosed herein may decrease excessive sleepiness or improve Karolinska Sleepiness Scale (KSS) rating in a subject in need thereof. In some embodiments, the KSS rating is improved 1, 2, 3, 4, or 5 or more ratings. In some embodiments, the subject has a KSS rating of 1, 2, 3, 4, or 5 after treatment with Compound (I).

The methods and uses disclosed herein may comprise performing one or more tests to quantify a subject's sleepiness. In some embodiments, the test is selected from the multiple sleep latency test (MSLT), maintenance of wakefulness test (MWT), and the Oxford Sleep Resistance (OSLER) test. In some embodiments, the test is MWT. In some embodiments, the testis the Karolinska Sleepiness Scale (KSS), the Epworth Sleepiness Scale (ESS), the Stanford Sleepiness Scale, Ullanlinna Narcolepsy Scale (UNS), Work Limitations Questionnaire (WLQ), SF-8 (subset of SF-36 questionnaire) or a combination thereof.

Modes of Administration

The methods and uses disclosed herein comprise administering Compound (I) to a subject in need thereof. In some embodiments, Compound (I) is administered orally. In some embodiments, Compound (I) is administered non-orally. In some embodiments, the non-oral administration is intravenous administration, subcutaneous administration, transdermal administration, intradermal administration or transmucosal administration. In some embodiments, the non-oral administration is intravenous administration. In some embodiments, the non-oral administration is subcutaneous administration. In some embodiments, the non-oral administration is transdermal administration. In some embodiments, Compound (I) is administered intravenously. Alternatively, or additionally, Compound (I) may be administered as an infusion. Administering Compound (I) as an infusion may comprise administering Compound (I) through a needle or catheter.

Compound (I) can be administered orally and non-orally such as intramuscular, intraperitoneal, intravenous, intraarterial, intraventricular, intracisternal injection or infusion; subcutaneous injection; or implant; or inhalation spray, intratracheal, nasal, vaginal, rectal, subdermal, transdermal, intradermal, epidural, ocular insert or ocular instillation administration, in a suitable unit dosage form containing a pharmaceutically acceptable conventional nontoxic carrier, adjuvant and vehicle suitable for each administration route.

In some embodiments, Compound (I) is administered as an infusion for at least 10, 20, 30, 40, 50, 60, 70, 80, 90, 100, 120, 140, 160, or 180 or more minutes. Compound (I) may be administered as an infusion for at least 1, 2, 3, 4, 5, 6, 7, 8, 9, 10, 11, 12, 13, 14, 15, 16, 17, 18, 19, or 20 or more hours. Compound (I) may be administered as an infusion for at least 2 hours. In some embodiments, the total time for administering Compound (I) is consecutive (e.g., Compound (I) is administered as an infusion for at least 2 consecutive hours). Alternatively, the total time for administering Compound (I) is intermittent (e.g., Compound (I) is administered as an infusion for 1 hour, then the infusion is stopped for period of time, and the infusion is restarted for another hour).

Alternatively, or additionally, administering Compound (I) may comprise administering an effective amount of Compound (I). In some embodiments, administering Compound (I) may comprise administering a therapeutically effective amount of Compound (I). The effective amount of Compound (I) may be between about 3 mg and about 500 mg. The effective amount of Compound (I) may be between about 5 mg and about 400 mg. The effective amount of Compound (I) may be between about 5 mg and about 300 mg of Compound (I). The effective amount of Compound (I) may be between about 15 mg and about 500 mg. The effective amount of Compound (I) may be between about 15 mg and about 400 mg of Compound (I). The effective amount of Compound (I) may be between about 15 mg and about 300 mg of Compound (I). The effective amount of Compound (I) may be between about 20 mg and about 500 mg of Compound (I). The effective amount of Compound (I) may be between about 20 mg and about 400 mg of Compound (I). The effective amount of Compound (I) may be between about 20 mg and about 300 mg of Compound (I). The effective amount of Compound (I) may be between about 30 mg and about 500 mg of Compound (I). The effective amount of Compound (I) may be between about 30 mg and about 400 mg of Compound (I). The effective amount of Compound (I) may be between about 30 mg and about 300 mg of Compound (I). The effective amount of Compound (I) may be between about 40 mg and about 500 mg of Compound (I). The effective amount of Compound (I) may be between about 40 mg and about 400 mg of Compound (I). The effective amount of Compound (I) may be between about 40 mg and about 300 mg of Compound (I). The effective amount of Compound (I) may be between about 40 mg and about 200 mg of Compound (I).

The effective amount of Compound (I) may be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, or 200 mg. The effective amount of Compound (I) may be at least 3 mg. The effective amount of Compound (I) may be at least 4 mg. The effective amount of Compound (I) may be at least 5 mg. The effective amount of Compound (I) may be at least 6 mg. The effective amount of Compound (I) may be at least 7 mg. The effective amount of Compound (I) may be at least 10 mg. The amount of Compound (I) may be at least 15 mg. The effective amount of Compound (I) may be at least 20 mg. The effective amount of Compound (I) may be at least 30 mg. The effective amount of Compound (I) may be at least 40 mg. The effective amount of Compound (I) may be at least 50 mg. The effective amount of Compound (I) may be at least 60 mg. The effective amount of Compound (I) may be at least 70 mg. The effective amount of Compound (I) may be at least 80 mg. The effective amount of Compound (I) may be at least 90 mg. The effective amount of Compound (I) may be at least 100 mg.

The effective amount of Compound (I) may be less than 550, 500, 450, 400, 350, 300, 290, 280, 275, 270, 260, 250, 240, 230, 225, 220, 210, 200, 175, 150, 125 or 100 mg. The effective amount of Compound (I) may be less than 500 mg. The effective amount of Compound (I) may be less than 450 mg. The effective amount of Compound (I) may be less than 400 mg. The effective amount of Compound (I) may be less than 350 mg. The effective amount of Compound (I) may be less than 300 mg. The effective amount of Compound (I) may be less than 250 mg. The effective amount of Compound (I) may be less than 200 mg. The effective amount of Compound (I) may be less than 150 mg. The effective amount of Compound (I) may be less than 100 mg.

The effective amount of Compound (I) may be between about 5 and about 500 mg. The effective amount of Compound (I) may be between about 5 and about 450 mg. The effective amount of Compound (I) may be between about 5 and about 400 mg. The effective amount of Compound (I) may be between about 5 and about 350 mg. The effective amount of Compound (I) may be between about 5 and about 300 mg. The effective amount of Compound (I) may be between about 5 and about 250 mg. The effective amount of Compound (I) may be between about 5 and about 200 mg. The effective amount of Compound (I) may be between about 5 and about 150 mg. The effective amount of Compound (I) may be between about 5 and about 100 mg.

The effective amount of Compound (I) may be between about 7 and about 500 mg. The effective amount of Compound (I) may be between about 7 and about 450 mg. The effective amount of Compound (I) may be between about 7 and about 400 mg. The effective amount of Compound (I) may be between about 7 and about 350 mg. The effective amount of Compound (I) may be between about 7 and about 300 mg. The effective amount of Compound (I) may be between about 7 and about 250 mg. The effective amount of Compound (I) may be between about 7 and about 200 mg. The effective amount of Compound (I) may be between about 7 and about 150 mg. The effective amount of Compound (I) may be between about 7 and about 100 mg.

The effective amount of Compound (I) may be between about 10 and about 500 mg. The effective amount of Compound (I) may be between about 10 and about 450 mg. The effective amount of Compound (I) may be between about 10 and about 400 mg. The effective amount of Compound (I) may be between about 10 and about 350 mg. The effective amount of Compound (I) may be between about 10 and about 300 mg. The effective amount of Compound (I) may be between about 10 and about 250 mg. The effective amount of Compound (I) may be between about 10 and about 200 mg. The effective amount of Compound (I) may be between about 10 and about 150 mg. The effective amount of Compound (I) may be between about 10 and about 100 mg.

The effective amount of Compound (I) may be between about 20 and about 500 mg. The effective amount of Compound (I) may be between about 20 and about 450 mg. The effective amount of Compound (I) may be between about 20 and about 400 mg. The effective amount of Compound (I) may be between about 20 and about 350 mg. The effective amount of Compound (I) may be between about 20 and about 300 mg. The effective amount of Compound (I) may be between about 20 and about 250 mg. The effective amount of Compound (I) may be between about 20 and about 200 mg. The effective amount of Compound (I) may be between about 20 and about 150 mg. The effective amount of Compound (I) may be between about 20 and about 100 mg.

The effective amount of Compound (I) may be between about 30 and about 500 mg. The effective amount of Compound (I) may be between about 30 and about 450 mg. The effective amount of Compound (I) may be between about 30 and about 400 mg. The effective amount of Compound (I) may be between about 30 and about 350 mg. The effective amount of Compound (I) may be between about 30 and about 300 mg. The effective amount of Compound (I) may be between about 30 and about 250 mg. The effective amount of Compound (I) may be between about 30 and about 200 mg. The effective amount of Compound (I) may be between about 30 and about 150 mg. The effective amount of Compound (I) may be between about 30 and about 100 mg.

The effective amount of Compound (I) may be between about 40 and about 500 mg. The effective amount of Compound (I) may be between about 40 and about 450 mg. The effective amount of Compound (I) may be between about 40 and about 400 mg. The effective amount of Compound (I) may be between about 40 and about 350 mg. The effective amount of Compound (I) may be between about 40 and about 300 mg. The effective amount of Compound (I) may be between about 40 and about 250 mg. The effective amount of Compound (I) may be between about 40 and about 200 mg. The effective amount of Compound (I) may be between about 40 and about 150 mg. The effective amount of Compound (I) may be between about 40 and about 100 mg.

The effective amount of Compound (I) may be between about 50 and about 500 mg. The effective amount of Compound (I) may be between about 50 and about 450 mg. The effective amount of Compound (I) may be between about 50 and about 400 mg. The effective amount of Compound (I) may be between about 50 and about 350 mg. The effective amount of Compound (I) may be between about 50 and about 300 mg. The effective amount of Compound (I) may be between about 50 and about 250 mg. The effective amount of Compound (I) may be between about 50 and about 200 mg. The effective amount of Compound (I) may be between about 50 and about 150 mg. The effective amount of Compound (I) may be between about 50 and about 100 mg.

The effective amount of Compound (I) may be between about 60 and about 500 mg. The effective amount of Compound (I) may be between about 60 and about 450 mg. The effective amount of Compound (I) may be between about 60 and about 400 mg. The effective amount of Compound (I) may be between about 60 and about 350 mg. The effective amount of Compound (I) may be between about 60 and about 300 mg. The effective amount of Compound (I) may be between about 60 and about 250 mg. The effective amount of Compound (I) may be between about 60 and about 200 mg. The effective amount of Compound (I) may be between about 60 and about 150 mg. The effective amount of Compound (I) may be between about 60 and about 100 mg.

The effective amount of Compound (I) may be between about 70 and about 500 mg. The effective amount of Compound (I) may be between about 70 and about 450 mg. The effective amount of Compound (I) may be between about 70 and about 400 mg. The effective amount of Compound (I) may be between about 70 and about 350 mg. The effective amount of Compound (I) may be between about 70 and about 300 mg. The effective amount of Compound (I) may be between about 70 and about 250 mg. The effective amount of Compound (I) may be between about 70 and about 200 mg. The effective amount of Compound (I) may be between about 70 and about 150 mg. The effective amount of Compound (I) may be between about 70 and about 100 mg.

The effective amount of Compound (I) may be between about 80 and about 500 mg. The effective amount of Compound (I) may be between about 80 and about 450 mg. The effective amount of Compound (I) may be between about 80 and about 400 mg. The effective amount of Compound (I) may be between about 80 and about 350 mg. The effective amount of Compound (I) may be between about 80 and about 300 mg. The effective amount of Compound (I) may be between about 80 and about 250 mg. The effective amount of Compound (I) may be between about 80 and about 200 mg. The effective amount of Compound (I) may be between about 80 and about 150 mg. The effective amount of Compound (I) may be between about 80 and about 100 mg.

The effective amount of Compound (I) may be between about 90 and about 500 mg. The effective amount of Compound (I) may be between about 90 and about 450 mg. The effective amount of Compound (I) may be between about 90 and about 400 mg. The effective amount of Compound (I) may be between about 90 and about 350 mg. The effective amount of Compound (I) may be between about 90 and about 300 mg. The effective amount of Compound (I) may be between about 90 and about 250 mg. The effective amount of Compound (I) may be between about 90 and about 200 mg. The effective amount of Compound (I) may be between about 90 and about 150 mg. The effective amount of Compound (I) may be between about 90 and about 100 mg.

The effective amount of Compound (I) may be between about 100 and about 500 mg. The effective amount of Compound (I) may be between about 100 and about 450 mg. The effective amount of Compound (I) may be between about 100 and about 400 mg. The effective amount of Compound (I) may be between about 100 and about 350 mg. The effective amount of Compound (I) may be between about 100 and about 300 mg. The effective amount of Compound (I) may be between about 100 and about 250 mg. The effective amount of Compound (I) may be between about 100 and about 200 mg. The effective amount of Compound (I) may be between about 100 and about 150 mg.

Depending on the subject and the period for administration of Compound (I), the effective amount may change. In some embodiments, the effective amount of Compound (I) may be gradually increased during the administration period of Compound (I) for the purpose of performing the intended or desired effect, or achieving the same or desired plasma concentration for Compound (I). In some embodiments, the effective amount of Compound (I) is gradually increased within the range from about 20 mg and about 500 mg of Compound (I). In some embodiments, the effective amount of Compound (I) is gradually increased within the range from about 20 mg and about 400 mg of Compound (I). In some embodiments, the effective amount of Compound (I) is gradually increased within the range from about 20 mg and about 300 mg of Compound (I). In some embodiments, the effective amount of Compound (I) is gradually increased within the range from about 20 mg and about 200 mg of Compound (I). In some embodiments, the effective amount of Compound (I) is gradually increased within the range from about 20 mg and about 100 mg of Compound (I).

In some embodiments, the effective amount of Compound (I) is gradually increased within the range from about 30 mg and about 500 mg of Compound (I). In some embodiments, the effective amount of Compound (I) is gradually increased within the range from about 30 mg and about 400 mg of Compound (I). In some embodiments, the effective amount of Compound (I) is gradually increased within the range from about 30 mg and about 300 mg of Compound (I). In some embodiments, the effective amount of Compound (I) is gradually increased within the range from about 30 mg and about 200 mg of Compound (I). In some embodiments, the effective amount of Compound (I) is gradually increased within the range from about 30 mg and about 100 mg of Compound (I).

In some embodiments, the effective amount of Compound (I) is gradually increased within the range from about 40 mg and about 500 mg of Compound (I). In some embodiments, the effective amount of Compound (I) is gradually increased within the range from about 40 mg and about 400 mg of Compound (I). In some embodiments, the effective amount of Compound (I) is gradually increased within the range from about 40 mg and about 300 mg of Compound (I). In some embodiments, the effective amount of Compound (I) is gradually increased within the range from about 40 mg and about 200 mg of Compound (I). In some embodiments, the effective amount of Compound (I) is gradually increased within the range from about 40 mg and about 100 mg of Compound (I).

In some embodiments, the effective amount of Compound (I) is gradually increased within the range from about 50 mg and about 500 mg of Compound (I). In some embodiments, the effective amount of Compound (I) is gradually increased within the range from about 50 mg and about 400 mg of Compound (I). In some embodiments, the effective amount of Compound (I) is gradually increased within the range from about 50 mg and about 300 mg of Compound (I). In some embodiments, the effective amount of Compound (I) is gradually increased within the range from about 50 mg and about 200 mg of Compound (I). In some embodiments, the effective amount of Compound (I) is gradually increased within the range from about 50 mg and about 100 mg of Compound (I).

In some embodiments, the effective amount of Compound (I) is gradually increased within the range from about 60 mg and about 500 mg of Compound (I). In some embodiments, the effective amount of Compound (I) is gradually increased within the range from about 60 mg and about 400 mg of Compound (I). In some embodiments, the effective amount of Compound (I) is gradually increased within the range from about 60 mg and about 300 mg of Compound (I). In some embodiments, the effective amount of Compound (I) is gradually increased within the range from about 60 mg and about 200 mg of Compound (I). In some embodiments, the effective amount of Compound (I) is gradually increased within the range from about 60 mg and about 100 mg of Compound (I).

In some embodiments, the effective amount of Compound (I) is gradually increased within the range from about 70 mg and about 500 mg of Compound (I). In some embodiments, the effective amount of Compound (I) is gradually increased within the range from about 70 mg and about 400 mg of Compound (I). In some embodiments, the effective amount of Compound (I) is gradually increased within the range from about 70 mg and about 300 mg of Compound (I). In some embodiments, the effective amount of Compound (I) is gradually increased within the range from about 70 mg and about 200 mg of Compound (I). In some embodiments, the effective amount of Compound (I) is gradually increased within the range from about 70 mg and about 100 mg of Compound (I).

In some embodiments, the effective amount of Compound (I) is gradually increased within the range from about 80 mg and about 500 mg of Compound (I). In some embodiments, the effective amount of Compound (I) is gradually increased within the range from about 80 mg and about 400 mg of Compound (I). In some embodiments, the effective amount of Compound (I) is gradually increased within the range from about 80 mg and about 300 mg of Compound (I). In some embodiments, the effective amount of Compound (I) is gradually increased within the range from about 80 mg about 200 mg of Compound (I). In some embodiments, the effective amount of Compound (I) is gradually increased within the range from about 80 mg and about 100 mg of Compound (I).

In some embodiments, the effective amount of Compound (I) is gradually increased within the range from about 90 mg and about 500 mg of Compound (I). In some embodiments, the effective amount of Compound (I) is gradually increased within the range from about 90 mg and about 400 mg of Compound (I). In some embodiments, the effective amount of Compound (I) is gradually increased within the range from about 90 mg and about 300 mg of Compound (I). In some embodiments, the effective amount of Compound (I) is gradually increased within the range from about 90 mg and about 200 mg of Compound (I). In some embodiments, the effective amount of Compound (I) is gradually increased within the range from about 90 mg and about 100 mg of Compound (I).

In some embodiments, the effective amount of Compound (I) is gradually increased within the range from about 100 mg and about 500 mg of Compound (I). In some embodiments, the effective amount of Compound (I) is gradually increased within the range from about 100 mg and about 400 mg of Compound (I). In some embodiments, the effective amount of Compound (I) is gradually increased within the range from about 100 mg and about 300 mg of Compound (I). In some embodiments, the effective amount of Compound (I) is gradually increased within the range from about 100 mg and about 200 mg of Compound (I).

### PK Parameters

The plasma concentration for Compound (I) may be about 50.90 ng/mL or more for about 1 hours or more.

The plasma concentration for Compound (I) may be about 38.21 ng/mL or more for about 1 hours or more.

The plasma concentration for Compound (I) may be at least 38.21, 42.08, 43.98, 50.76, 50.90, 60.54, 64.43, 74.04, 75.67, 77.96, 85.03, 87.54, 89.17, 89.28, 98.66, 99.99, 102.89, 103.38, 112.29, 117.59, 162.56, 163.57, 187.06, 194.71, 210.06, 210.55, 217.61, 219.04, 225.13, 234.06, 240.51, 253.39, 258.06 or 287.74 ng/mL for at least 1 hours. The plasma concentration for Compound (I) may be at least 38.21, 42.08, 43.98, 50.76, 50.90, 60.54, 64.43, 74.04, 75.67, 77.96, 85.03, 87.54, 89.17, 89.28, 98.66, 99.99, 102.89, 103.38, 112.29, 117.59, 162.56, 163.57, 187.06, 194.71, 210.06, 210.55, 217.61, 219.04, 225.13, 234.06, 240.51, 253.39, 258.06 or 287.74 ng/mL for at least 2 hours. The plasma concentration for Compound (I) may be at least 38.21, 42.08, 43.98, 50.76, 50.90, 60.54, 64.43, 74.04, 75.67, 77.96, 85.03, 87.54, 89.17, 89.28, 98.66, 99.99, 102.89, 103.38, 112.29, 117.59, 162.56, 163.57, 187.06, 194.71, 210.06, 210.55, 217.61, 219.04, 225.13, 234.06, 240.51, 253.39, 258.06 or 287.74 ng/mL for at least 4 hours. The plasma concentration for Compound (I) may be at least 38.21, 42.08, 43.98, 50.76, 50.90, 60.54, 64.43, 74.04, 75.67, 77.96, 85.03, 87.54, 89.17, 89.28, 98.66, 99.99, 102.89, 103.38, 112.29, 117.59, 162.56, 163.57, 187.06, 194.71, 210.06, 210.55, 217.61, 219.04, 225.13, 234.06, 240.51, 253.39, 258.06 or 287.74 ng/mL for at least 6 hours. The plasma concentration for Compound (I) may be at least 38.21, 42.08, 43.98, 50.76, 50.90, 60.54, 64.43, 74.04, 75.67, 77.96, 85.03, 87.54, 89.17, 89.28, 98.66, 99.99, 102.89, 103.38, 112.29, 117.59, 162.56, 163.57, 187.06, 194.71, 210.06, 210.55, 217.61, 219.04, 225.13, 234.06, 240.51, 253.39, 258.06 or 287.74 ng/mL for at least 8 hours. The plasma concentration for Compound (I) may be at least 38.21, 42.08, 43.98, 50.76, 50.90, 60.54, 64.43, 74.04, 75.67, 77.96, 85.03, 87.54, 89.17, 89.28, 98.66, 99.99, 102.89, 103.38, 112.29, 117.59, 162.56, 163.57, 187.06, 194.71, 210.06, 210.55, 217.61, 219.04, 225.13, 234.06, 240.51, 253.39, 258.06 or 287.74 ng/mL for at least 12 hours.

The plasma concentration for Compound (I) may be at least 25, 30, 35, 40, 45, 50, 55, 60, 65, 70, 75, 80, 85, 90, 95, 100, 105, 110, 115, 120, 125, 130, 135, 140, 145, 150, 160, 170, 180, 190, 200, 210, 220, 230, 240, 250, 300, 310, 320, 330, 340 or 350 ng/mL for at least 1 hours. The plasma concentration for Compound (I) may be at least 25, 30, 35, 40, 45, 50, 55, 60, 65, 70, 75, 80, 85, 90, 95, 100, 105, 110, 115, 120, 125, 130, 135, 140, 145, 150, 160, 170, 180, 190, 200, 210, 220, 230, 240, 250, 300, 310, 320, 330, 340 or 350 ng/mL for at least 2 hours. The plasma concentration for Compound (I) may be at least 25, 30, 35, 40, 45, 50, 55, 60, 65, 70, 75, 80, 85, 90, 95, 100, 105, 110, 115, 120, 125, 130, 135, 140, 145, 150, 160, 170, 180, 190, 200, 210, 220, 230, 240, 250, 300, 310, 320, 330, 340 or 350 ng/mL for at least 4 hours. The plasma concentration for Compound (I) may be at least 25, 30, 35, 40, 45, 50, 55, 60, 65, 70, 75, 80, 85, 90, 95, 100, 105, 110, 115, 120, 125, 130, 135, 140, 145, 150, 160, 170, 180, 190, 200, 210, 220, 230, 240, 250, 300, 310, 320, 330, 340 or 350 ng/mL for at least 6 hours. The plasma concentration for Compound (I) may be at least 25, 30, 35, 40, 45, 50, 55, 60, 65, 70, 75, 80, 85, 90, 95, 100, 105, 110, 115, 120, 125, 130, 135, 140, 145, 150, 160, 170, 180, 190, 200, 210, 220, 230, 240, 250, 300, 310, 320, 330, 340 or 350 ng/mL for at least 8 hours. The plasma concentration for Compound (I) may be at least 25, 30, 35, 40, 45, 50, 55, 60, 65, 70, 75, 80, 85, 90, 95, 100, 105, 110, 115, 120, 125, 130, 135, 140, 145, 150, 160, 170, 180, 190, 200, 210, 220, 230, 240, 250, 300, 310, 320, 330, 340 or 350 ng/mL for at least 12 hours.

The plasma concentration for Compound (I) may be between 38.21 and 500 ng/mL for at least 1, 2, 4, 6, 8 or 12 hours. The plasma concentration for Compound (I) may be between 38.21 and 400 ng/mL for at least 1, 2, 4, 6, 8 or 12 hours. The plasma concentration for Compound (I) may be between 38.21 and 300 ng/mL for at least 1, 2, 4, 6, 8 or 12 hours. The plasma concentration for Compound (I) may be between 38.21 and 200 ng/mL for at least 1, 2, 4, 6, 8 or 12 hours.

The plasma concentration for Compound (I) may be between 50.90 and 500 ng/mL for at least 1, 2, 4, 6, 8 or 12 hours. The plasma concentration for Compound (I) may be between 50.90 and 400 ng/mL for at least 1, 2, 4, 6, 8 or 12 hours. The plasma concentration for Compound (I) may be between 50.90 and 300 ng/mL for at least 1, 2, 4, 6, 8 or 12 hours. The plasma concentration for Compound (I) may be between 50.90 and 200 ng/mL for at least 1, 2, 4, 6, 8 or 12 hours.

The plasma concentration for Compound (I) may be between 60.54 and 500 ng/mL for at least 1, 2, 4, 6, 8 or 12 hours. The plasma concentration for Compound (I) may be between 60.54 and 400 ng/mL for at least 1, 2, 4, 6, 8 or 12 hours. The plasma concentration for Compound (I) may be between 60.54 and 300 ng/mL for at least 1, 2, 4, 6, 8 or 12 hours. The plasma concentration for Compound (I) may be between 60.54 and 200 ng/mL for at least 1, 2, 4, 6, 8 or 12 hours.

The plasma concentration for Compound (I) may be between 64.43 and 500 ng/mL for at least 1, 2, 4, 6, 8 or 12 hours. The plasma concentration for Compound (I) may be between 64.43 and 400 ng/mL for at least 1, 2, 4, 6, 8 or 12 hours. The plasma concentration for Compound (I) may be between 64.43 and 300 ng/mL for at least 1, 2, 4, 6, 8 or 12 hours. The plasma concentration for Compound (I) may be between 64.43 and 200 ng/mL for at least 1, 2, 4, 6, 8 or 12 hours.

The plasma concentration for Compound (I) may be between 74.04 and 500 ng/mL for at least 1, 2, 4, 6, 8 or 12 hours. The plasma concentration for Compound (I) may be between 74.04 and 400 ng/mL for at least 1, 2, 4, 6, 8 or 12 hours. The plasma concentration for Compound (I) may be between 74.04 and 300 ng/mL for at least 1, 2, 4, 6, 8 or 12 hours. The plasma concentration for Compound (I) may be between 74.04 and 200 ng/mL for at least 1, 2, 4, 6, 8 or 12 hours.

The plasma concentration for Compound (I) may be between 77.96 and 500 ng/mL for at least 1, 2, 4, 6, 8 or 12 hours. The plasma concentration for Compound (I) may be between 77.96 and 400 ng/mL for at least 1, 2, 4, 6, 8 or 12 hours. The plasma concentration for Compound (I) may be between 77.96 and 300 ng/mL for at least 1, 2, 4, 6, 8 or 12 hours. The plasma concentration for Compound (I) may be between 77.96 and 200 ng/mL for at least 1, 2, 4, 6, 8 or 12 hours.

The plasma concentration for Compound (I) may be between 87.54 and 500 ng/mL for at least 1, 2, 4, 6, 8 or 12 hours. The plasma concentration for Compound (I) may be between 87.54 and 400 ng/mL for at least 1, 2, 4, 6, 8 or 12 hours. The plasma concentration for Compound (I) may be between 87.54 and 300 ng/mL for at least 1, 2, 4, 6, 8 or 12 hours. The plasma concentration for Compound (I) may be between 87.54 and 200 ng/mL for at least 1, 2, 4, 6, 8 or 12 hours.

The plasma concentration for Compound (I) may be between 89.28 and 500 ng/mL for at least 1, 2, 4, 6, 8 or 12 hours. The plasma concentration for Compound (I) may be between 89.28 and 400 ng/mL for at least 1, 2, 4, 6, 8 or 12 hours. The plasma concentration for Compound (I) may be between 89.28 and 300 ng/mL for at least 1, 2, 4, 6, 8 or 12 hours. The plasma concentration for Compound (I) may be between 89.28 and 200 ng/mL for at least 1, 2, 4, 6, 8 or 12 hours.

The plasma concentration for Compound (I) may be between 98.66 and 500 ng/mL for at least 1, 2, 4, 6, 8 or 12 hours. The plasma concentration for Compound (I) may be between 98.66 and 400 ng/mL for at least 1, 2, 4, 6, 8 or 12 hours. The plasma concentration for Compound (I) may be between 98.66 and 300 ng/mL for at least 1, 2, 4, 6, 8 or 12 hours. The plasma concentration for Compound (I) may be between 98.66 and 200 ng/mL for at least 1, 2, 4, 6, 8 or 12 hours.

The plasma concentration for Compound (I) may be between 103.38 and 500 ng/mL for at least 1, 2, 4, 6, 8 or 12 hours. The plasma concentration for Compound (I) may be between 103.38 and 400 ng/mL for at least 1, 2, 4, 6, 8 or 12 hours. The plasma concentration for Compound (I) may be between 103.38 and 300 ng/mL for at least 1, 2, 4, 6, 8 or 12 hours. The plasma concentration for Compound (I) may be between 103.38 and 200 ng/mL for at least 1, 2, 4, 6, 8 or 12 hours.

The plasma concentration for Compound (I) may be between 150 and 500 ng/mL for at least 1, 2, 4, 6, 8 or 12 hours. The plasma concentration for Compound (I) may be between 150 and 400 ng/mL for at least 1, 2, 4, 6, 8 or 12 hours. The plasma concentration for Compound (I) may be between 150 and 300 ng/mL for at least 1, 2, 4, 6, 8 or 12 hours. The plasma concentration for Compound (I) may be between 150 and 200 ng/mL for at least 1, 2, 4, 6, 8 or 12 hours.

The plasma concentration for Compound (I) may be between 162.56 and 500 ng/mL for at least 1, 2, 4, 6, 8 or 12 hours. The plasma concentration for Compound (I) may be between 162.56 and 400 ng/mL for at least 1, 2, 4, 6, 8 or 12 hours. The plasma concentration for Compound (I) may be between 162.56 and 300 ng/mL for at least 1, 2, 4, 6, 8 or 12 hours. The plasma concentration for Compound (I) may be between 162.56 and 200 ng/mL for at least 1, 2, 4, 6, 8 or 12 hours.

In some embodiments, the plasma concentration for Compound (I) represents an average plasma concentration level for a group of treated subjects and the time period of 1 hours or more begins at any time point following administration. In some embodiments, the plasma concentration for Compound (I) of about 50.90 ng/ml or more for a period of about 1 hour or more represents an average plasma concentration level for a group of treated subjects and the time period of 1 hour or more begins at any time point following administration. As long as the average plasma concentration for a group of treated subjects meets a condition, “about 50.90 ng/ml or more for a period of about 1 hour or more”, the plasma concentration for the individually treated subject may deviate from the condition. Such deviation is still within the scope of the appended claims.

In some embodiments, the plasma concentration for Compound (I) represents the plasma concentration level for the individually treated subject and the time period of 1 hour or more begins at any time point following administration to that subject. In some embodiments, the plasma concentration for Compound (I) of about 50.90 ng/ml or more for a period of about 1 hour or more represents the plasma concentration level for the individually treated subject and the time period of 1 hour or more begins at any time point following administration to that subject.

The Cmax for administration of Compound (I) may be about 94.66 ng/mL or more. The Cmax for administration of Compound (I) may be at least 70, 75, 80, 85, 90, 94.66, 95, 100, 105, 106.4, 110, 115, 118.1, 120, 125, 130,135, 140, 145, 150, 160, 170, 180, 190, 200, 210, 220, 228.3, 230, 240, 250, 260, 268.4, 270, 280, 290, 300, 308.5, 310, 320, 330, 340 or 350 ng/mL. The Cmax for administration of Compound (I) may be at least 106.4 ng/mL. The Cmax for administration of Compound (I) may be at least 118.1 ng/mL. The Cmax for administration of Compound (I) may be at least 268.4 ng/mL.

The Cmax for administration of Compound (I) may be about 600 ng/mL or less. The Cmax for administration of Compound (I) may be at most 600, 550, 500, 450 or 400 ng/mL.

In some embodiments, the plasma concentration for Compound (I) is maintained at about 150 ng/mL or more.

In some embodiments, the further plasma concentration for Compound (I) is about a half of Cmax for administration of Compound (I) or less at about 1 hour prior to sleep time. In some embodiments, the further plasma concentration for Compound (I) is about a quarter of Cmax for administration of Compound (I) or less at about 1 hour prior to sleep time.

In some embodiments, the further plasma concentration for Compound (I) is about a half of 50.90 ng/mL or less at about 1 hour prior to sleep time. In some embodiments, the further plasma concentration for Compound (I) is about a quarter of 50.90 ng/mL or less at about 1 hour prior to sleep time.

The AUC∞ for administration of Compound (I) may be about 829 ng*h/mL or more. The AUC∞ for administration of Compound (I) may be at least 600, 650, 700, 750, 800, 829, 850, 900, 950, 963.7, 1000, 1098.4, 1100, 1200, 1300, 1400, 1500, 1600, 1700, 1800, 1900, 2000, 2100, 2183.7, 200, 2300, 2368.7, 2400, 2500, 2553.7, 2600, 2700, 2800, 2900 or 3000 ng*h/mL. The AUC∞ for administration of Compound (I) may be at least 963.7 ng*h/mL. The AUC∞ for administration of Compound (I) may be at least 1098.4 ng*h/mL. The AUC∞ for administration of Compound (I) may be at least 2368.7 ng*h/mL.

The AUC∞ for administration of Compound (I) may be about 5000 ng*h/mL or less. The AUC∞ for administration of Compound (I) may be at most 6000, 5500, 5000, 4500, 4000 or 3500, ng*h/mL.

### Frequency of Administration

Compound (I) may be administered at least 1, 2, 3, 4, 5, 6, 7, 8, 9, 10 or more times per day. In some embodiments, Compound (I) is administered at least once per day. In some embodiments, Compound (I) is administered at least twice per day.

Compound (I) may be administered at least 1, 2, 3, 4, 5, 6, 7, 8, 9, 10, 11, 12, 13, or 14 or more times per week. In some embodiments, Compound (I) is administered at once per week. In some embodiments, Compound (I) is administered at least twice per week. In some embodiments, Compound (I) is administered at least 3 times per week. In some embodiments, Compound (I) is administered at least 4 times per week.

Compound (I) may be administered at least 1, 2, 3, 4, 5, 6, 7, 8, 9, 10, 11, 12, 13, 14, 15, 16, 17, 18, 19, or 20 or more times per month. Compound (I) may be administered at least 4 times per month.

### Combination Therapy

The methods disclosed herein may further comprise administering one or more additional therapies. The kits and compositions disclosed herein may further comprise one or more additional therapies. The one or more additional therapies may be selected from a stimulant, an antidepressant, a central nervous system depressant, a histamine 3 (H3) receptor antagonist, and any one of the concomitant drugs described hereinafter. In some embodiments, the stimulant is modafinil. In some embodiments, the antidepressant is clomipramine. In some embodiments, the central nervous system depressant is sodium oxybate. In some embodiments, the one or more additional therapies is venlafaxine or desvenlafaxine. In some embodiments, the H3 receptor antagonist is pitolisant.

Examples of the concomitant drug include, but are not limited to, the following. A therapeutic drug for narcolepsy (e.g., methylphenidate, amphetamine, pemoline, phenelzine, protriptyline, sodium oxybate, modafinil, caffeine, pitolisant, solriamfertol), antiobesity drug (amphetamine, benzfetamine, bromocriptine, bupropion, diethylpropion, exenatide, fenfluramine, liothyronine, liraglutide, mazindol, methamphetamine, octreotide, octreotide, orlistat, phendimetrazine, phendimetrazine, phenmetrazine, phentermine, Qnexa (registered trade mark), phenylpropanolamine, pramlintide, propylhexedrine, recombinant leptin, sibutramine, topiramate, zimelidine, zonisamide, Lorcaserin, metformin), acetylcholine esterase inhibitor (e.g., donepezil, rivastigmine, galanthamine, zanapezil, idebenone, tacrine), antidementia agent (e.g., memantine), inhibitor of β-amyloid protein production, secretion, accumulation, aggregation and/or deposition, β-secretase inhibitor (e.g., 6-(4-biphenylyl) methoxy-2-[2-(N,N-dimethylamino)ethyl]tetralin, 6-(4-biphenylyl)methoxy-2-(N,N-dimethyl amino)methyl-tetralin, 6-(4-biphenylyl)methoxy-2-(N,N-dipropylamino) methyltetralin, 2-(N,N-dimethylamino)methyl-6-(4′-methoxybiphenyl-4-yl)methoxytetralin, 6-(4-biphenylyl) methoxy-2-[2-(N,N-diethylamino)ethyl]tetralin, 2-[2-(N,N-dimethylamino)ethyl]-6-(4′-methylbiphenyl-4-yl)methoxytetralin, 2-[2-(N,N-dimethylamino)ethyl]-6-(4′-methoxy biphenyl-4-yl)methoxytetralin, 6-(2′,4′-dimethoxybiphenyl-4-yl)methoxy-2-[2-(N,N-dimethylamino)ethyl]tetralin, 6-[4-(1,3-benzodioxol-5-yl)phenyl]methoxy-2-[2-(N,N-dimethylamino)ethyl]tetralin, 6-(3′,4′-dimethoxybiphenyl-4-yl)methoxy-2-[2-(N,N-dimethylamino)ethyl]tetralin, an optically active form thereof, a salt thereof and a hydrate thereof, OM99-2 (WO01/00663)), γ-secretase inhibitor, β-amyloid protein aggregation inhibitor (e.g., PTI-00703, ALZHEMED (NC-531), PPI-368 (National Publication of International Patent Application No. 11-514333), PPI-558 (National Publication of International Patent Application No. 2001-500852), SKF-74652 (Biochem. J. (1999), 340(1), 283-289)), β-amyloid vaccine, β-amyloid-degrading enzyme and the like, brain function enhancer (e.g., aniracetam, nicergoline), therapeutic drug for Parkinson's disease [(e.g., dopamine receptor agonist (e.g., L-DOPA, bromocriptine, pergolide, talipexole, pramipexole, cabergoline, amantadine), monoamine oxidase enzyme (MAO) inhibitor (e.g., deprenyl, selegiline, remacemide, riluzole), anticholinergic agent (e.g., trihexyphenidyl, biperiden), COMT inhibitor (e.g., entacapone)], therapeutic drug for amyotrophic lateral sclerosis (e.g., riluzole etc., neurotrophic factor), therapeutic drug for abnormal behavior accompanying progress of dementia, wandering and the like (e.g., sedative, anti-anxiety drug), apoptosis inhibitor (e.g., CPI-1189, IDN-6556, CEP-1347), neuronal differentiation/regenerate promoter (e.g., leteprinim, xaliproden; SR-57746-A), SB-216763, Y-128, VX-853, prosaptide, 5,6-dimethoxy-2-[2,2,4,6,7-pentamethyl-3-(4-methylphenyl)-2,3-dihydro-1-benzofuran-5-yl]isoindoline, 5,6-dimethoxy-2-[3-(4-isopropylphenyl)-2,2,4,6,7-pentamethyl-2,3-dihydro-1-benzofuran-5-yl]isoindoline, 6-[3-(4-isopropylphenyl)-2,2,4,6,7-pentamethyl-2,3-dihydro-1-benzofuran-5-yl]-6,7-dihydro-5H-[1,3]dioxolo[4,5-f]isoindole and an optically active form, salt or hydrate thereof), non-steroidal antiinflammatory agents (meloxicam, tenoxicam, indomethacin, ibuprofen, celecoxib, rofecoxib, aspirin, indomethacin etc.), steroid drug (dexamethasone, hexestrol, cortisone acetate etc.), disease-modifying anti-rheumatic drug (DMARDs), anti-cytokine drug (e.g., TNF inhibitor, MAP kinase inhibitor), therapeutic agent for incontinence, frequent urination (e.g., flavoxate hydrochloride, oxybutynin hydrochloride, propiverine hydrochloride), phosphodiesterase inhibitor (e.g., sildenafil(citrate)), dopamine agonist (e.g., apomorphine), antiarrhythmic drugs (e.g., mexiletine), sex hormone or a derivative thereof (e.g., progesterone, estradiol, estradiol benzoate), therapeutic agent for osteoporosis (e.g., alfacalcidol, calcitriol, elcatonin, calcitonin salmon, estriol, ipriflavone, pamidronate disodium, alendronate sodium hydrate, incadronate disodium), parathyroid hormone (PTH), calcium receptor antagonists, therapeutic drug for insomnia (e.g., benzodiazepines medicament, non-benzodiazepines medicament, melatonin agonist, orexin receptor antagonists), therapeutic drug for schizophrenia (e.g., typical antipsychotic agents such as haloperidol and the like; atypical antipsychotic agents such as clozapine, olanzapine, risperidone, aripiprazole and the like; medicament acting on metabotropic glutamate receptor or ion channel conjugated-type glutamate receptor; phosphodiesterase inhibitor), benzodiazepines medicament (chlordiazepoxide, diazepam, potassium clorazepate, lorazepam, clonazepam, alprazolam etc.), L-type calcium channel inhibitor (pregabalin etc.), tricyclic or tetracyclic antidepressant (imipramine hydrochloride, amitriptyline hydrochloride, desipramine hydrochloride, clomipramine hydrochloride etc.), selective serotonin reuptake inhibitor (fluvoxamine maleate, fluoxetine hydrochloride, citalopram hydrobromide, sertraline hydrochloride, paroxetine hydrochloride, escitalopram oxalate etc.), serotonin-noradrenaline reuptake inhibitor (venlafaxine hydrochloride, duloxetine hydrochloride, desvenlafaxine hydrochloride etc.), noradrenaline reuptake inhibitor (reboxetine mesylate etc.), mirtazapine, trazodone hydrochloride, nefazodone hydrochloride, bupropion hydrochloride, setiptiline maleate, 5-HT_{1A} agonist, (buspirone hydrochloride, tandospirone citrate, osemozotan hydrocloride etc.), 5-HT_{2A} antagonist, 5-HT_{2A} inverse agonist, 5-HT₃ antagonist (cyamemazine etc.), heart non-selective R inhibitor (propranolol hydrochloride, oxprenolol hydrochloride etc.), histamine H₁ antagonist (hydroxyzine hydrochloride etc.), CRF antagonist, other antianxiety drug (meprobamate etc.), tachykinin antagonist (MK-869, saredutant etc.), medicament that acts on metabotropic glutamate receptor, CCK antagonist, β3 adrenaline antagonist (amibegron hydrochloride etc.), GAT-1 inhibitor (tiagabine hydrochloride etc.), N-type calcium channel inhibitor, carbonic anhydrase II inhibitor, NMDA glycine moiety agonist, NMDA antagonist (memantine etc.), peripheral benzodiazepine receptor agonist, vasopressin antagonist, vasopressin V1b antagonist, vasopressin V1a antagonist, phosphodiesterase inhibitor, opioid antagonist, opioid agonist, uridine, nicotinic acid receptor agonist, thyroid hormone (T3, T4), TSH, TRH, MAO inhibitor (phenelzine sulfate, tranylcypromine sulfate, moclobemide etc.), COMT inhibitor (entacapone etc.), therapeutic drug for bipolar disorder (lithium carbonate, sodium valproate, lamotrigine, riluzole, felbamate etc.), cannabinoid CB1 antagonist (rimonabant etc.), FAAH inhibitor, sodium channel inhibitor, anti-ADHD drug (methylphenidate hydrochloride, methamphetamine hydrochloride etc.), therapeutic drug for alcoholism, therapeutic drug for autism, therapeutic drug for chronic fatigue syndrome, therapeutic drug for spasm, therapeutic drug for fibromyalgia syndrome, therapeutic drug for headache, therapeutic drug for quitting smoking, therapeutic drug for myasthenia gravis, therapeutic drug for cerebral infarction, therapeutic drug for mania, therapeutic drug for hypersomnia, therapeutic drug for pain, therapeutic drug for dysthymia, therapeutic drug for autonomic ataxia, therapeutic drug for male and female sexual dysfunction, therapeutic drug for migraine, therapeutic drug for pathological gambler, therapeutic drug for restless legs syndrome, therapeutic drug for substance addiction, therapeutic drug for alcohol-related syndrome, therapeutic drug for irritable bowel syndrome, therapeutic drug for ALS (riluzole etc., neurotrophic factor etc.), therapeutic drug for lipid abnormality such as cholesterol-lowering drug (statin series (pravastatin sodium, atorvastatin, simvastatin, rosuvastatin etc.), fibrate (clofibrate etc.), squalene synthetase inhibitor), therapeutic drug for abnormal behavior or suppressant of dromomania due to dementia (sedatives, antianxiety drug etc.), antiobesity drug, therapeutic drug for diabetes, therapeutic agent for diabetic complications, therapeutic drug for hypertension, therapeutic drug for hypotension, diuretic, chemotherapeutic agent, immunotherapeutic agent, antithrombotic agent, anti-cancer agent and the like.

Two or more kinds of the above-mentioned concomitant drug may be used in a mixture at an appropriate ratio.

Compound (I) can also be used in combination with biologics (e.g., antibody drug, nucleic acid or nucleic acid derivative, aptamer drug, vaccine preparation), or can be combined with a gene therapy method and the like and applied as a combination therapy, or can also be used in combination with a treatment in psychiatric field without using drugs.

Examples of the treatment method in the psychiatric field without using drug include modified electroconvulsive therapy, deep brain stimulation therapy, repetitive transcranial magnetic stimulation therapy, psychotherapy including cognitive behavioral therapy and the like.

### Pharmaceutical Compositions

Further disclosed herein are pharmaceutical compositions comprising Compound (I). In some embodiments, the pharmaceutical composition comprises (a) methyl 3-((methylsulfonyl)amino)-2-(((4-phenylcyclohexyl)oxy)methyl)piperidine-1-carboxylate (Compound (I)), or a salt thereof, and (b) a pharmaceutically acceptable carrier therefor.

In some embodiments, the pharmaceutical composition provides a plasma concentration for Compound (I) of about 50.90 ng/mL or more. In some embodiments, the pharmaceutical composition provides a Cmax for Compound (I) of about 94.66 ng/mL or more. In some embodiments, the pharmaceutical composition AUC∞ for Compound (I) of about 829 ng*h/mL or more. In some embodiments, the pharmaceutical composition an effective amount of Compound (I). Examples of the effective amount include between about 3 mg and about 500 mg, between about 5 mg and about 300 mg, and between about 5 mg and about 100 mg. The effective amount is preferably between about 5 mg and about 50 mg.

In some embodiments, the compound (I) is methyl (2R,3S)-3-((methylsulfonyl)amino)-2-(((cis-4-phenylcyclohexyl)oxy)methyl)piperidine-1-carboxylate (Compound A).

The pharmaceutically acceptable carrier may be a cyclodextrin. The cyclodextrin may be betadex sulfobutyl ether sodium.

As pharmaceutically acceptable carriers, various organic or inorganic carrier substances conventionally used as preparation materials can be used. These are incorporated as excipient, lubricant, binder and disintegrant for solid preparations; or solvent, solubilizing agent, suspending agent, isotonicity agent, buffer and soothing agent for liquid preparations; and the like; and preparation additives such as preservative, antioxidant, colorant, sweetening agent and the like can be added as necessary.

Examples of the dosage form of the aforementioned pharmaceutical composition include tablet (including sugar-coated tablet, film-coated tablet, orally disintegrating tablet), capsule (including soft capsule, microcapsule), granule, powder, troche, syrup, emulsion, suspension, films (e.g., orally disintegrable films), injection (e.g., subcutaneous injection, intravenous injection, intramuscular injection, intraperitoneal injection, drip infusion), external preparation (e.g., dermal preparation, ointment), suppository (e.g., rectal suppository, vaginal suppository), pellet, nasal preparation, pulmonary preparation (inhalant), eye drop and the like, which can be respectively safely administered orally or non-orally (e.g., topical, rectal, intravenous administration). These preparations may be a release control preparation (e.g., sustained-release microcapsule) such as an immediate-release preparation, a sustained-release preparation and the like.

In some embodiments, the pharmaceutical composition is formulated for oral administration. In some embodiments, the pharmaceutical composition is formulated for non-oral administration. In some embodiments, the pharmaceutical composition is formulated for intravenous administration, subcutaneous administration, transdermal administration, intradermal administration or transmucosal administration. In some embodiments, the pharmaceutical composition is formulated for intravenous administration. In some embodiments, the pharmaceutical composition is formulated for subcutaneous administration. In some embodiments, the pharmaceutical composition is formulated for transdermal administration.

### Optically Active Compound

In some embodiments, Compound (I) is an optically active compound. In some embodiments, Compound (I) is methyl (2R,3S)-3-((methylsulfonyl)amino)-2-(((cis-4-phenylcyclohexyl)oxy)methyl)piperidine-1-carboxylate (Compound A) in any of the methods, the uses and the pharmaceutical compositions disclosed herein. Compound (I) including its salt and its optical active compound may be produced as disclosed in WO2017/135306.

### Kits

Further disclosed herein are kits comprising Compound (I). In some embodiments, the kit comprises (a) a container comprising methyl 3-((methylsulfonyl)amino)-2-(((4-phenylcyclohexyl)oxy)methyl)piperidine-1-carboxylate (Compound (I)), or a salt thereof, and (b) instructions for administering Compound (I).

The kits disclosed herein may further comprise an additional container comprising saline.

The container may be a glass vial. Alternatively, the container may be a syringe.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

### Definitions

Unless defined otherwise, all technical and scientific terms used herein have the meaning commonly understood by a person skilled in the art to which this disclosure belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

As used herein, the singular forms “a”, “an” and “the” are intended to include the plural forms as well, unless the context clearly indicates otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “administration” of an agent to a subject includes any route of introducing or delivering the agent to a subject to perform its intended function. Administration can be carried out by any suitable non-oral route, including, but not limited to, intravenously, intramuscularly, intraperitoneally, subcutaneously, and other suitable routes as described herein. Administration includes self-administration and the administration by another.

As used herein, the term “effective amount” or “therapeutically effective amount” refers to a quantity of Compound (I) sufficient to achieve a desired effect or a desired therapeutic effect. In the context of therapeutic applications, the amount of Compound (I) administered to the subject can depend on the type and severity of the disease or symptom and on the characteristics of the individual, such as general health, age, sex, body weight and tolerance to drugs. The skilled artisan will be able to determine appropriate dosages depending on these and other factors.

As used herein, the term “modulate” refers positively or negatively alter. Exemplary modulations include an about 1%, about 2%, about 5%, about 10%, about 25%, about 50%, about 75%, or about 100% change.

As used herein, the term “increase” refers to alter positively by at least about 5%, including, but not limited to, alter positively by about 5%, by about 10%, by about 25%, by about 30%, by about 50%, by about 75%, or by about 100%.

As used herein, the term “reduce” refers to alter negatively by at least about 5% including, but not limited to, alter negatively by about 5%, by about 10%, by about 25%, by about 30%, by about 50%, by about 75%, or by about 100%.

As used herein, the term “an OX2R agonist” refers to methyl 3-((methylsulfonyl)amino)-2-(((4-phenylcyclohexyl)oxy)methyl)piperidine-1-carboxylate (“Compound (I)”), or a salt thereof. In some embodiments, Compound (I) is methyl (2R,3S)-3-((methylsulfonyl)amino)-2-(((cis-4-phenylcyclohexyl)oxy)methyl)piperidine-1-carboxylate (Compound A).

### EXAMPLES

The following non-limiting examples are put forth so as to provide those of ordinary skill in the art with a complete disclosure and description of how to make and use the compositions, and assay, screening, and therapeutic methods of the invention, and are not intended to limit the scope of what the inventors regard as their invention.

### Example I-1: Human Study of Safety/Tolerability, Pharmacokinetics and Pharmacodynamics of a Single Dose of an Orexin 2 Receptor (OX2R) Agonist in Sleep-Deprived Healthy Adults

Primary Objective

The primary objective of this study was to determine the effect of Compound A (methyl (2R,3S)-3-((methylsulfonyl)amino)-2-(((cis-4-phenylcyclohexyl)oxy)methyl)piperidine-1-carboxylate), 44 mg (low dose [LD]) and 112 mg (high dose [HD]), after a single IV dose (compared to placebo) on promoting wakefulness as measured by sleep latency on the MWT (performed at approximately 2, 4, 6, and 8 hours post-dosing starting at approximately 1:00 AM and then at approximate times of 3:00, 5:00, and 7:00 AM) in sleep-deprived healthy volunteers.

Secondary Objectives

The secondary objectives of the study were as follows:
1. To assess the safety/tolerability and pharmacokinetic (PK) parameters of a single IV infusion of Compound A in sleep-deprived healthy volunteers.
2. To determine the effect of a single dose of modafinil (300 mg) on promoting wakefulness as measured by sleep latency on the MWT to confirm assay sensitivity.
3. To evaluate the effect of Compound A on a measure of sleepiness, as compared to placebo.

Exploratory Objectives

Exploratory objectives in sleep-deprived healthy volunteers were as follows:
1. To compare the effects of Compound A (LD and HD) and modafinil (300 mg) on promoting wakefulness as measured by MWT.
2. To determine PK-PD relationships for selected PD measures.
3. To evaluate PK-BP relationships in BP change after Compound A administration.
4. To compare various polysomnography (PSG) parameters between Day −1 (predose) and Day 2 (recovery sleep).
5. To determine whether there is any difference in the PSG parameters on Day 2 (recovery sleep) between Compound A and placebo or modafinil.
6. To evaluate the continuous electroencephalogram (EEG) obtained during the MWTs for power spectra changes for Compound A (LD and HD) versus placebo, Compound A (LD and HD) versus modafinil, and modafinil versus placebo.

Overall Study Design and Plan

This was a phase 1b, single-center, randomized, double-blind, double-dummy, placebo- and active-controlled, 4-period Williams design crossover study to evaluate the PK, PD, and safety of Compound A in sleep-deprived healthy volunteers. Compound A was administered as a 9-hour IV infusion.

Healthy adult male subjects aged 18 to 40 years, inclusive, who satisfied the inclusion and exclusion criteria were enrolled in the trial. On Day 1 of treatment period 1, eligible subjects were equally randomized to 4 treatment sequence groups (sequence 1 to 4) which defined the order of administration for Compound A LD, Compound A HD (both delivered as a 9-hour IV infusion), modafinil 300 mg, and placebo.

A summary of the study drug assignment and sequence in presented in Table 1.

TABLE 1
Summary of Study Drug Assignment and Treatment
Sequence (Double Dummy for Placebo)

| Sequence | N | Period 1 | Period 2 | Period 3 | Period 4 |
|---|---|---|---|---|---|
| 1 | 5 | Compound | Placebo | Compound | Modafinil |
| | | A 44 mg | | A 112 mg | 300 mg |
| | | (LD) | | (HD) | |
| 2 | 5 | Compound | Compound | Modafinil | Placebo |
| | | A 112 mg | A 44 mg | 300 mg | |
| | | (HD) | (LD) | | |
| 3 | 5 | Modafinil | Compound | Placebo | Compound |
| | | 300 mg | A 112 mg | | A 44 mg |
| | | | (HD) | | (LD) |
| 4 | 5 | Placebo | Modafinil | Compound | Compound |
| | | | 300 mg | A 44 mg | A 112 mg |
| | | | | (LD) | (HD) |

HD: high dose, Compound A, 112 mg; LD: low dose, Compound A, 44 mg.

At the screening visit, subjects completed medical examinations, an electrocardiogram (ECG), and clinical laboratory tests. After the screening visit, eligible participants wore an actigraph from Day −6 until Day −1. After validation that actigraphy results showed a normal sleep-wake cycle and subjects had a negative drug screen, subjects underwent an 8-hour nocturnal polysomnography (NPSG) to exclude any pre-existing sleep disorders. Actigraphy results were also collected for the 5-night period before Day −1 (Day −6 to Day −1) for every treatment period to ensure that sleep fell within normal nocturnal times as defined in the following: the subject had regular sleep-wake habits (e.g., routinely spent 6.5-8 hours sleeping nightly, not oversleeping by more than 3 hours [total sleep not more than 11 hours]) as determined by investigator interviews and confirmed in 5-day actigraphy records and whom regularly fell asleep between 9:30 PM and 12:00 AM.

During the day before dosing on Day 1, participants were administered the KSS at scheduled timepoints. A practice MWT session was performed on Day 1 treatment period 1 (only once during the entire study) to familiarize subjects with the procedures. Study drug was administered in the clinic in the evening on Day 1 of each treatment period. Subjects underwent the MWT and KSS at specified timepoints after the start of the infusion. Subjects were required to stay awake between the MWT tests. Following completion of the IV infusion on Day 2, subjects underwent an additional MWT test. When the final MWT test and cognitive testing were completed, subjects were allowed to sleep (recovery sleep) for approximately 6 hours. PSG recording was collected during this time. Subjects were discharged from the unit after completion on Day 2 with continuing actigraphy upon discharge (to begin on Day −6 before the next treatment period). The interval between each subsequent treatment period was at least 7 days to ensure that the subject's circadian rhythm had returned to a normal cycle. Subject's vital signs, including BP, were monitored during the dosing and testing period. Blood samples for determination of Compound A plasma concentrations were collected at specified timepoints on Days 1 and 2 of each treatment period. Subjects completed the Columbia-Suicide Severity Rating Scale (C-SSRS) during screening, before study drug administration, and before discharge on Day 2 of each treatment period.

This was a 4-period crossover study. Each subject checked in to the clinical site in the afternoon or early evening on Day −1 of a treatment period and left after completion of all study-related procedures on Day 2. At the discretion of the investigator, subjects could be allowed to remain at the clinical site longer.

The study ended when the last subject completed the last planned or follow-up visit/interaction associated with a planned visit (this could be a phone contact), discontinued from the study, or was lost to follow-up (i.e., the investigator was unable to contact the subject).

An overview of the study schedule is provided in Table 2.

TABLE 2
Overview of Study Schedule

| | | Treatment Period | | Time Interval |
|---|---|---|---|---|
| Screening | Check-in and | Dose/Sample | | Between Subsequent |
| Period | Baseline | Collection | Sample Collection | Treatment Period |
| Days −28 | Day −1 | Day 1 Sleep | Day 2 Sleep | Minimum of 7 days |
| to −2 | NPSG/BP | deprivation/study | deprivation/study | with actigraphy |
| | measurement ^{a} | drug administration/ | drug continued | for 5 days |
| | | study assessment | infusion/study | |
| | | | assessment/ | |
| | | | recovery sleep | |

X-------------------Confinement-------------------X

BP: blood pressure;
NPSG: nocturnal polysomnography
^{a} NPSG and time-matched BP only on Day −1 of treatment period 1. For periods 2, 3, and 4, subjects will be admitted to the unit and allowed to sleep normally without NPSG or time-matched BP measurement. The time interval from the end of 1 period to the start of the next period will be at least 7 days.

Diet and Fluids

On Day 1, study drug was administered beginning at approximately 9:45 PM for tablet (modafinil or placebo) and approximately 10:45 PM for IV infusion (Compound A or placebo) for each treatment period. The modafinil or matching placebo were given with 240 mL of water for oral (PO) administration. Subjects were no food or drink (NPO) for 2 hours after the PO modafinil/placebo administration.

Since study drug administration did not begin until evening, subjects had a standard breakfast, lunch, and dinner on Day 1. Snacks were given until approximately 8:30 PM on the day of dosing (Day 1). No other food was consumed until breakfast on Day 2. Subjects fasted from all food and drink except water between meals and snacks. The caloric content and composition of meals was the same for all subjects in each treatment period. On Day 2, breakfast was provided after the fourth MWT, KSS, and cognitive testing were completed. After the postdose procedures were completed, subsequent meals and snacks were unrestricted in caloric content, composition, and timing.

Subjects refrained from consuming mustard greens (i.e., kale, broccoli, watercress, collard greens, kohlrabi, Brussel sprouts, and mustard) and charbroiled meat 7 days before administration of the first dose of study drug, throughout the study (including the washout interval between treatment periods), and until the follow-up visit.

Activity

Subjects avoided unaccustomed strenuous physical activity (i.e., weight lifting, running, bicycling, etc.) from the screening visit until administration of the initial dose of study drug, throughout the study (including washout intervals between treatment periods), and until the follow-up visit.

On Day 1 of each period, starting at approximately 8:30 PM, the subject's activities were restricted so that they were not overstimulated or upset. Cell phone use was restricted from approximately 8:30 PM until after recovery sleep the next day. Activities permitted from approximately 8:30 PM until the start of recovery sleep were board games, puzzles, adult coloring books, drawing, reading, listening to music (not on cell phones), watching television or nonviolent movies, and walking and talking with other participants/staff. Subjects could have brought in small craft projects they were interested in or working on.

Endpoints and Criteria for Evaluation

Primary endpoint: Latency for each MWT, defined as time to sleep onset.

Secondary endpoints:
PK parameters calculated from plasma concentrations of Compound A.
Scores for sleepiness on the Karolinska Sleepiness Scale (KSS).

Safety endpoints: adverse events (AEs), physical examinations, vital signs, 12-lead electrocardiograms (ECGs), clinical laboratory evaluations (hematology, blood chemistry, and urinalysis), drug-liking visual analogue scale, and Profile of Mood States.

Study Population

Inclusion Criteria:
Be a nonsmoker who has not used tobacco- or nicotine-containing products (example, nicotine patch) for at least 6 months before study drug administration of the initial dose of study drug.
Have regular sleep-wake habits (example, routinely spending 6.5 to 8 hours sleeping nightly, not oversleeping by more than 3 hours on weekends, that is, total sleep not more than 11 hours) as determined by investigator interviews and confirmed in 5-day actigraphy records and whom regularly fall asleep between 9:30 PM and 12:00 AM.
Be willing to have actigraphy monitoring during the week before randomization and in each interval.

Exclusion Criteria:
Has a positive alcohol or drug screen.
Has a history of alcohol consumption exceeding 2 standard drinks per day on average (1 glass is approximately equivalent to: beer [354 milliliter per (mL/)12 ounces], wine (118 mL/4 ounces), or distilled spirits (29.5 mL/1 ounce)] per day).
Has excessive sleepiness defined by a self-reported Epworth Sleepiness Scale score at screening greater than 10; irregular work hours; or routine night-shift work within 1 month before randomization.
Currently experiencing or having a history of any known/suspected sleep disorder, any disorder associated with excessive daytime somnolence (EDS), or any diagnosis interfering with assessment of sleepiness.
Abnormal findings on the initial polysomnography (PSG) conducted on Day −1 (check-in), as specified in the study manual.
Traveled across 2 or more time zones 2 weeks or less before screening.
Caffeine consumption of more than 400 milligram per day (mg/day) for 2 weeks before screening (1 serving of coffee is approximately equivalent to 120 mg of caffeine).

### Example I-2: Human Study of Safety/Tolerability of an Orexin 2 Receptor (OX2R) Agonist in Sleep-Deprived Healthy Adults

Safety Conclusions

Incidence of AEs in the Compound A treatments was generally higher than in the placebo treatment, but most AEs were mild and no SAEs were observed.
No clinically significant changes from baseline in clinical laboratory evaluations, vital signs, physical examinations, or electrocardiograms were reported for any subject during the study. Drug-related TEAEs of liver function test increased were reported in 1 subject in Compound A 112 mg treatment and 1 subject in modafinil treatment; the events were mild and moderate in intensity, respectively.
Changes in DBP appeared to increase with increasing concentration of Compound A. There were no apparent trends in the change from time-matched baseline in HR or SBP when compared to Compound A plasma concentration. The effect on BP and pulse were similar between Compound A 112 mg and modafinil.
Overall single dose administration of Compound A 44 mg or Compound A 112 mg in a slow infusion over 9 hours in healthy volunteers was well-tolerated with no major safety concerns.

Brief Summary of AEs

There were no deaths or SAEs and no subject was discontinued from study drug due to an AE.

Overall, 18 subjects (90.0%) experienced TEAEs in placebo, Compound A (44 and 112 mg), and modafinil (300 mg) treatments. The majority of subjects experienced TEAEs that were considered related (15 subjects [75.0%]) to study drug and were of mild intensity (15 subjects [75.0%]).

### Example I-3: Compound a Single Dose PK in Healthy Subjects

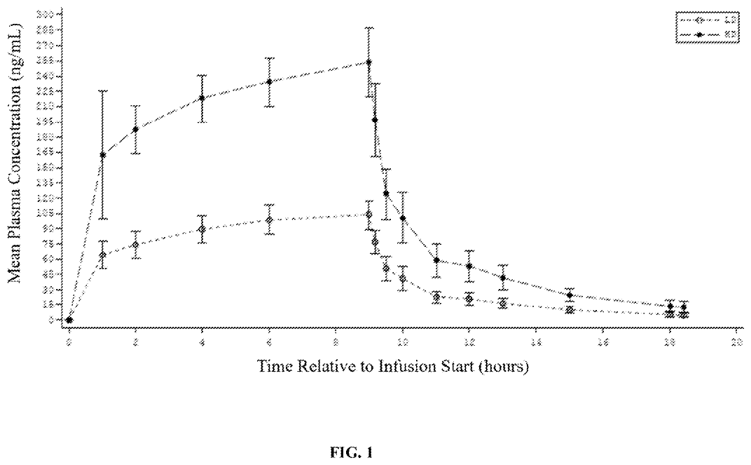

Plasma concentrations were tabulated, and descriptive statistics were calculated for each Compound A dose level and are listed in Tables 3-6 for Compound A. The mean plasma concentration versus time plots for Compound A are shown in FIG. 1.

TABLE 3
Descriptive Statistics of Compound A (44 mg) at Post Infusion Start
Pharmacokinetic Set
Regimen: Compound A 44 mg
Analyte: Compound A (ng/mL)

| | | Plasma Concentrations (ng/mL) at Each | | | | |
|---|---|---|---|---|---|---|
| | | Scheduled Collection Time | | | | |
| Summary | | Post Infusion Start (hours) | | | | |
| Statistics | Predose | 1 | 2 | 4 | 6 | 9 |
| N | 18 | 18 | 18 | 18 | 18 | 18 |
| Mean | 0.00 | 64.43 | 74.04 | 89.28 | 98.66 | 103.38 |
| SD | 0.000 | 13.531 | 13.498 | 13.609 | 13.632 | 14.214 |
| % CV | | 21.0 | 18.2 | 15.2 | 13.8 | 13.7 |
| Median | 0.00 | 65.55 | 72.60 | 88.25 | 97.40 | 101.00 |
| Min | 0 | 41 | 50.4 | 65 | 74.2 | 75.5 |
| Max | 0 | 95.6 | 100 | 113 | 127 | 132 |
| GM | | 63.09 | 72.88 | 88.29 | 97.76 | 102.46 |

TABLE 4
Descriptive Statistics of Compound A (44 mg) at Post Infusion End
Pharmacokinetic Set
Regimen: Compound A 44 mg
Analyte: Compound A (ng/mL)

| | Plasma Concentrations (ng/mL) at Each Scheduled | | | | | | | | |
|---|---|---|---|---|---|---|---|---|---|
| | Collection Time | | | | | | | | |
| Summary | Post Infusion End (hours) | | | | | | | | |
| Statistics | 0.17 | 0.5 | 1 | 2 | 3 | 4 | 6 | 9 | Discharge |
| N | 18 | 18 | 18 | 18 | 18 | 18 | 18 | 18 | 18 |
| Mean | 76.86 | 50.78 | 40.73 | 22.23 | 20.67 | 16.28 | 10.32 | 5.56 | 4.95 |
| SD | 11.913 | 11.796 | 12.227 | 6.228 | 6.231 | 4.688 | 2.997 | 2.651 | 2.015 |
| % CV | 15.5 | 23.2 | 30.0 | 28.0 | 30.2 | 28.8 | 29.1 | 47.7 | 40.7 |
| Median | 74.80 | 49.10 | 38.15 | 21.00 | 20.35 | 17.25 | 10.75 | 5.06 | 4.78 |
| Min | 62.4 | 31.6 | 21.9 | 12 | 11.1 | 7.94 | 4.92 | 2.13 | 1.98 |
| Max | 110 | 72.6 | 62.4 | 36.9 | 34.3 | 23.7 | 16.7 | 11.9 | 10.8 |
| GM | 76.07 | 49.49 | 39.05 | 21.44 | 19.74 | 15.56 | 9.85 | 5.06 | 4.60 |

TABLE 5
Descriptive Statistics of Compound A (112 mg) at Post Infusion Start
Pharmacokinetic Set
Regimen: Compound A 112 mg
Analyte: Compound A (ng/mL)

| | Plasma Concentrations (ng/mL) at Each Scheduled | | | | | |
|---|---|---|---|---|---|---|
| | Collection Time | | | | | |
| Summary | Post Infusion Start (hours) | | | | | |
| Statistics | Predose | 1 | 2 | 4 | 6 | 9 |
| N | 18 | 18 | 18 | 18 | 18 | 18 |
| Mean | 0.00 | 162.56 | 187.06 | 217.61 | 234.06 | 253.39 |
| SD | 0.000 | 62.568 | 23.493 | 99.902 | 24.004 | 34.346 |
| % CV | | 38.5 | 12.6 | 10.5 | 10.3 | 13.6 |
| Median | 0.00 | 151.00 | 179.50 | 210.00 | 229.50 | 252.50 |
| Min | 0 | 115 | 156 | 190 | 200 | 159 |
| Max | 0 | 397 | 233 | 273 | 283 | 319 |
| GM | | 155.56 | 185.71 | 216.56 | 232.92 | 250.96 |

TABLE 6
Descriptive Statistics of Compound A (112 mg) at Post Infusion End
Pharmacokinetic Set
Regimen: Compound A 112 mg
Analyte: Compound A (ng/mL)

| | Plasma Concentrations (ng/mL) at Each Scheduled | | | | | | | | |
|---|---|---|---|---|---|---|---|---|---|
| | Collection Time | | | | | | | | |
| Summary | Post Infusion End (hours) | | | | | | | | |
| Statistics | 0.17 | 0.5 | 1 | 2 | 3 | 4 | 6 | 9 | Discharge |
| N | 18 | 18 | 18 | 18 | 18 | 18 | 18 | 18 | 18 |
| Mean | 196.83 | 124.21 | 100.48 | 58.92 | 53.01 | 41.98 | 24.24 | 13.78 | 13.01 |
| SD | 35.384 | 24.742 | 24.484 | 16.068 | 14.923 | 12.427 | 6.110 | 4.968 | 5.117 |
| % CV | 18.0 | 19.9 | 24.4 | 27.3 | 28.2 | 29.6 | 25.2 | 36.0 | 39.3 |
| Median | 197.50 | 121.50 | 96.90 | 61.40 | 54.00 | 44.70 | 23.40 | 11.95 | 11.50 |
| Min | 128 | 69.8 | 53.1 | 30 | 28.1 | 26.4 | 17.1 | 7.01 | 6.29 |
| Max | 258 | 160 | 140 | 94.4 | 78.2 | 73.1 | 36.9 | 24.1 | 23.8 |
| GM | 193.73 | 121.68 | 97.47 | 56.70 | 50.80 | 40.36 | 23.57 | 13.02 | 12.09 |

The Compound A mean plasma concentration-time profiles appeared similar in the LD (44 mg) and HD (112 mg) groups. The mean plasma concentrations of Compound A increased gradually during IV infusion, reaching more than 80% of the observed maximum concentration by 4 hours after start of infusion. Following a single 9-hour IV infusion to healthy subjects, mean Compound A plasma concentrations were quantifiable up to the last sampling time point, subject discharge, which occurred approximately 9.5 hours after the end of infusion (18.5 hours postdose) in both the dose groups and increased approximately dose-proportionately with Compound A dose. A 2.55-fold increase in dose resulted in a 2.52-, 2.45-, 2.45- and 2.46-fold increase in Cₘₐₓ, Cₑₒᵢ, AUCₗₐₛₜ, and AUC_{∞}, respectively. After the IV infusion ended, Compound A plasma concentrations declined rapidly in a biphasic manner with a mean terminal half-life of approximately 3 hours.

Descriptive statistics for plasma PK parameter estimates of Compound A are summarized in Table 7.

TABLE 7
Summary of Compound A Plasma PK Parameter Estimates Following Administration of
a Single 9-hour IV Infusion of 44 or 112 mg Compound A in Healthy Male Subjects

| | | tₘₐₓ | Cₘₐₓ | Cₑₒᵢ | AUCₗₐₛₜ | AUC_{∞} | t_{1/2z} | CL | V_{z} | Vₛₛ |
|---|---|---|---|---|---|---|---|---|---|---|
| Dose | | (h) | (ng/mL) | (ng/mL) | (h*ng/mL) | (h*ng/mL) | (h) | (L/h) | (L) | (L) |
| 44 mg | N | 18 | 18 | 18 | 18 | 18 | 18 | 18 | 18 | 18 |
| [LD] | Mean | 8.154 | 106.4 | 103.4 | 940.4 | 963.7 | 3.134 | 46.4 | 207.4 | 106.7 |
| | SD | 1.4247 | 11.74 | 14.21 | 127.26 | 134.70 | 0.7064 | 5.98 | 41.51 | 18.90 |
| | Min | 5.92 | 90.7 | 75.5 | 770 | 779 | 2.29 | 34.4 | 140 | 81.1 |
| | Median | 9.000 | 102.0 | 101.0 | 887.5 | 911.0 | 3.020 | 48.3 | 211.0 | 99.2 |
| | Max | 9.03 | 132 | 132 | 1233 | 1280 | 4.71 | 56.5 | 298 | 149 |
| | CV % | 17.5 | 11.0 | 13.7 | 13.5 | 14.0 | 22.5 | 12.9 | 20.0 | 17.7 |
| | GM | | 105.8 | 102.5 | 932.7 | 955.3 | | | | |
| 112 mg | N | 18 | 18 | 18 | 18 | 18 | 18 | 18 | 18 | 18 |
| [HD] | Mean | 7.880 | 268.4 | 253.4 | 2303.7 | 2368.7 | 3.252 | 47.6 | 223.1 | 112.5 |
| | SD | 2.1430 | 40.10 | 34.35 | 174.23 | 185.04 | 0.8597 | 3.56 | 61.20 | 25.33 |
| | Min | 1.07 | 222 | 159 | 2065 | 2109 | 2.19 | 39.1 | 145 | 57.9 |
| | Median | 9.000 | 260.00 | 252.5 | 2297.5 | 2377.5 | 3.165 | 47.1 | 216.5 | 111.5 |
| | Max | 9.02 | 397 | 319 | 2732 | 2867 | 4.87 | 53.1 | 329 | 162 |
| | CV % | 27.2 | 14.9 | 13.6 | 7.6 | 7.8 | 26.4 | 7.5 | 27.4 | 22.5 |
| | GM | | 266.0 | 251.0 | 2297.6 | 2362.1 | | | | |

AUCₗₐₛₜ: area under the plasma concentration-time curve from 0 to time of the last quantifiable concentration; AUC_{∞}: area under the first moment concentration-time curve from time 0 to infinity; Cₑₒᵢ: plasma concentration at the end of infusion; CL: total clearance after intravenous administration; Cₘₐₓ: maximum observed plasma concentration; GM: geometric mean; HD: high dose, Compound A 112 mg; IV: intravenous; LD: low dose, Compound A 44 mg; PK: pharmacokinetic; t_{1/2Z}: terminal disposition phase half-life; tₘₐₓ: time of first occurrence of Cₘₐₓ; Vₛₛ: volume of distribution at steady state after intravenous administration; V_{z}: volume of distribution during the terminal disposition phase after intravenous administration.

### Example I-4: Sleep Latency in Maintenance of Wakefulness Test (MWT)

### Primary Efficacy Endpoint—MWT

Statistical analysis of the average latency to sleep onset is provided in Table 8.

TABLE 8
Statistical Analysis of Sleep Latency on the MWT during Infusion - Latency to
Sleep Onset
Pharmacodynamic Set
Parameter: Latency to Sleep Onset (min)

| | | Test | | Ref (Placebo) | | LS Mean Difference | | |
|---|---|---|---|---|---|---|---|---|
| | | LS Mean | | LS Mean | | Estimate | | |
| Test (N) | Ref (N) | (SE) | 95% CI | (SE) | 95% CI | (SE) | 95% CI | p-value |

Average Overall

| | | | | | | | | |
|---|---|---|---|---|---|---|---|---|
| HD | P | 38.82 | (34.92, | 8.61 | (4.95, | 30.21 | (24.85, | <.001*** |
| | | (1.955) | 42.72) | (1.836) | 12.28) | (2.682) | 35.56) | |
| LD | | 25.40 | (21.50, | | | 16.79 | (11.44, | <.001*** |
| | | (1.955) | 29.31) | | | (2.682) | 22.15) | |
| M | | 30.87 | (27.10, | | | 22.62 | (17.00, | <.001*** |
| | | (1.888) | 34.64) | | | (2.634) | 27.52) | |

2 HOURS POST INFUSION START

| | | | | | | | | |
|---|---|---|---|---|---|---|---|---|
| HD (18) | P (20) | 40.02 | (35.14, | 15.68 | (11.07, | 24.35 | (17.64, | <.001*** |
| | | (2.445) | 44.90) | (2.305) | 20.28) | (3.360) | 31.06) | |
| LD (18) | | 35.74 | (30.86, | | | 20.06 | (13.35, | <.001*** |
| | | (2.445) | 40.62) | | | (3.360) | 26.77) | |
| M (19) | | 35.57 | (30.84, | | | 19.89 | (13.30, | <.001*** |
| | | (2.368) | 40.30) | | | (3.304) | 26.49) | |

4 HOURS POST INFUSION START

| | | | | | | | | |
|---|---|---|---|---|---|---|---|---|
| HD (18) | P (20) | 40.05 | (35.42, | 9.10 | (4.74, | 30.95 | (24.59, | <.001*** |
| | | (2.320) | 44.68) | (2.186) | 13.46) | (3.187) | 37.31) | |
| ED (18) | | 32.04 | (27.41, | | | 22.94 | (16.58, | <.001*** |
| | | (2.320) | 36.67) | | | (3.187) | 29.30) | |
| M (19) | | 35.60 | (31.11, | | | 26.50 | (20.24, | <.001*** |
| | | (2.246) | 40.08) | | | (3.134) | 32.75) | |

6 HOURS POST INFUSION START

| | | | | | | | | |
|---|---|---|---|---|---|---|---|---|
| HD (18) | P (20) | 38.36 | (32.89, | 6.15 | (0.99, | 32.21 | (24.68, | <.001*** |
| | | (2.742) | 43.83) | (2.589) | 11.31) | (3.771) | 39.73) | |
| LD (18) | | 20.71 | (15.24, | | | 14.56 | (7.04, | <.001** |
| | | (2.742) | 26.18) | | | (3.771) | 22.08) | |
| M(19) | | 31.89 | (26.58, | | | 25.74 | (18.33, | <.001*** |
| | | (2.659) | 37.19) | | | (3.711) | 33.14) | |

8 HOURS POST INFUSION START

| | | | | | | | | |
|---|---|---|---|---|---|---|---|---|
| HD (18) | P (20) | 36.86 | (32.31, | 3.53 | (-0.76, | 33.33 | (27.08, | <.001*** |
| | | (2.279) | 41.41) | (2.146) | 7.81) | (3.131) | 39.58) | |
| LD (18) | | 13.13 | (8.58, | | | 9.60 | (3.35, | 0.003* |
| | | (2.279) | 17.68) | | | (3.131) | 15.85) | |
| M(19) | | 20.44 | (16.03, | | | 16.91 | (10.77, | <.001*** |
| | | (2.206) | 24.84) | | | (3.078) | 23.06) | |

Note:
*,**,***indicate significance at 0.05, 0.001, and 0.0001 level, respectively.
Note:
Sleep latency was analyzed using a linear mixed effects model with fixed effects for sequence, period, treatment, time (as categorical variable) and treatment-by-time interaction, and a random effect for subject.
LD: Compound A 44 mg; HD: Compound A 112 mg; M: Modafinil 300 mg; P: Placebo.
Program Source\T BIOMWT1.sas (PRAHS SAS V9.4)

As shown in Table 9, the LS Mean difference (95% CI) from placebo in the average sleep latency from 4 MWT sessions post modafinil 300 mg dose was 22.26 (17.00, 27.52) min. It is statistically significant at level of 0.05. Statistical significance was also observed for the difference in the LS means of sleep latency from placebo in each of the 4 MWT sessions (Table 8). Assay sensitivity for a clinically meaningful PD effect was established in this study.

The LS Mean differences (95% CI) from placebo in average sleep latency from 4 MWT sessions post infusion start were 16.79 (11.44, 22.15) and 30.21 (24.85, 35.56) minutes for Compound A 44 mg and Compound A 112 mg, respectively. Both differences were statistically significant. Both treatments were also statistically significantly different from placebo at 2, 4, 6, and 8 hours post infusion start, respectively.

TABLE 9
Sleep Latency on the MWT Post Infusion Start

| | | Test | LS Mean Difference | | |
|---|---|---|---|---|---|
| Test | N | LS Mean (SE) | Estimate (SE) | 95% CI | p-value |

Average Overall

| | | | | | |
|---|---|---|---|---|---|
| Placebo | 20 | 8.61 (1.836) | | | |
| Compound A 44 mg | 18 | 25.40 (1.955) | 16.79 (2.682) | (11.44, 22.15) | <0.001*** |
| Compound A 112 mg | 18 | 38.82 (1.955) | 30.21 (2.682) | (24.85, 35.56) | <0.001*** |
| Modafinil 300 mg | 19 | 30.87 (1.888) | 22.26 (2.634) | (17.00, 27.52) | <0.001*** |

CI: confidence interval; LS: least squares; MWT: maintenance of wakefulness test; SE: standard error.
***indicates significance at the 0.0001 level.

Latency to sleep onset on MWT post end of infusion data is summarized in Table 10. As the table shows, modafinil 300 mg still has a 16 minutes delay to sleep onset 1 hour post end of infusion relative to placebo (p-value<0.001). However, the effect of the Compound A on sleep latency has diminished 1 hour post end of infusion.

TABLE 10
Latency to Sleep Onset on the MWT After the End of Infusion

| | | Test | LS Mean Difference | | |
|---|---|---|---|---|---|
| Test | N | LS Mean (SE) | Estimate (SE) | 95% CI | p-value |
| Placebo | 20 | 4.65 (2.109) | | | |
| Compound A 44 mg | 18 | 2.49 (2.241) | −2.16 (2.752) | (−7.68, 3.36) | 0.436 |
| Compound A 112 mg | 18 | 2.36 (2.241) | −2.29 (2.752) | (−7.81, 3.23) | 0.409 |
| Modafinil 300 mg | 19 | 21.42 (2.168) | 16.77 (2.692) | (11.37, 22.18) | <.001*** |

CI: confidence interval; MWT: maintenance of wakefulness test; LS: least squares; SE: standard error.
***indicates significance at the 0.0001 level.

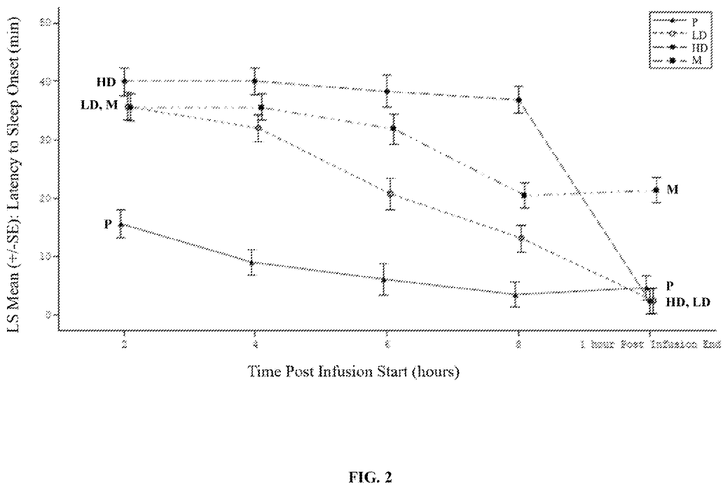

A graphical representation of the latency to sleep onset data of LS mean (±SE) of latency to sleep onset vs time is presented in FIG. 2.

Additional MT Endpoints

Statistical analyses of additional MWT endpoints are provided in Tables 11-16. These results are not adjusted for multiple comparisons.

TABLE 11
Statistical Analysis of Additional MWT Endpoints during Infusion -
Duration Total Microsleeps
Pharmacodynamic Set
Parameter: Duration Total Microsleeps (sec)

| | | Test | | Ref (Placebo) | | LS Mean Difference | | |
|---|---|---|---|---|---|---|---|---|
| | | LS Mean | | LS Mean | | Estimate | | |
| Test (N) | Ref (N) | (SE) | 95% CI | (SE) | 95% CI | (SE) | 95% CI | p-value |

Average Overall

| | | | | | | | | |
|---|---|---|---|---|---|---|---|---|
| HD | P | 1.40 | (−3.54, | 9.08 | (4.42, | −7.68 | (−14.5, | 0.027* |
| | | (2.476) | 6.34) | (2.331) | 13.73) | (3.400) | −0.89) | |
| LD | | 8.89 | (3.95, | | | −0.18 | (−6.97, | 0.957 |
| | | (2.476) | 13.83) | | | (3.400) | 6.61) | |
| M | | 9.00 | (4.21, | | | −0.08 | (−6.75, | 0.982 |
| | | (2.396) | 13.78) | | | (3.342) | 6.60) | |

2 HOURS POST INFUSION START

| | | | | | | | | |
|---|---|---|---|---|---|---|---|---|
| HD (18) | P (20) | −0.02 | (−5.09, | 8.80 | (4.02, | −8.82 | (−15.8, | 0.014* |
| | | (2.540) | 5.05) | (2.393) | 13.58) | (3.490) | −1.85) | |
| ED (18) | | 1.61 | (−3.46, | | | −7.19 | (−14.2, | 0.043* |
| | | (2.540) | 6.68) | | | (3.490) | −0.22) | |
| M (19) | | 5.75 | (0.84, | | | −3.05 | (−9.90, | 0.377 |
| | | (2.459) | 10.66) | | | (3.431) | 3.80) | |

4 HOURS POST INFUSION START

| | | | | | | | | |
|---|---|---|---|---|---|---|---|---|
| HD (18) | P (20) | −0.02 | (−7.69, | 12.30 | (5.04, | −12.3 | (−22.9, | 0.023* |
| | | (3.846) | 7.66) | (3.637) | 19.56) | (5.293) | −1.75) | |
| LD (18) | | 10.39 | (2.72, | | | −1.91 | (−12.5, | 0.720 |
| | | (3.846) | 18.06) | | | (5.293) | 8.65) | |
| M (19) | | 7.91 | (0.45, | | | −4.39 | (−14.8, | 0.402 |
| | | (3.734) | 15.36) | | | (5.213) | 6.01) | |

6 HOURS POST INFUSION START

| | | | | | | | | |
|---|---|---|---|---|---|---|---|---|
| HD (18) | P (20) | 0.43 | (−6.26, | 7.40 | (1.08, | −6.97 | (−16.2, | 0.135 |
| | | (3.351) | 7.11) | (3.166) | 13.72) | (4.610) | 2.23) | |
| LD (18) | | 11.84 | (5.15, | | | 4.44 | (−4.76, | 0.339 |
| | | (3.351) | 18.52) | | | (4.610) | 13.63) | |
| M (19) | | 9.70 | (3.21, | | | 2.30 | (−6.76, | 0.615 |
| | | (3.251) | 16.18) | | | (4.538) | 11.35) | |

8 HOURS POST INFUSION START

| | | | | | | | | |
|---|---|---|---|---|---|---|---|---|
| HD (18) | P (20) | 5.21 | (−2.97, | 7.80 | (0.07, | −2.59 | (−13.9, | 0.647 |
| | | (4.097) | 13.38) | (3.876) | 15.53) | (5.640) | 8.66) | |
| ED (18) | | 11.72 | (3.55, | | | 3.92 | (−7.33, | 0.489 |
| | | (4.097) | 19.90) | | | (5.640) | 15.18) | |
| M (19) | | 12.64 | (4.70, | | | 4.84 | (−6.24, | 0.386 |
| | | (3.979) | 20.58) | | | (5.555) | 15.92) | |

Note:
*,**,***indicate significance at 0.05, 0.001, and 0.0001 level, respectively.
Note:
MWT parameters were analyzed using a linear mixed effects model with fixed effects for sequence, period, treatment, time (as categorical variable) and treatment-by-time interaction, and a random effect for subject.
Note:
Stage N3 Duration and Stage REM Duration were not included in statistical analyses due to the lack of variability in measurements.
LD: Compound A 44 mg; HD: Compound A 112 mg; M: Modafinil 300 mg; P: Placebo.
Program Source\T_BIOMWT2.sas (PRAHS SAS V9.4)

TABLE 12
Statistical Analysis of Additional MWT Endpoints during
Infusion - Stage N1 Duration
Pharmacodynamic Set
Parameter: Stage N1 Duration (min)

| | | Test | | Ref (Placebo) | | LS Mean Difference | | |
|---|---|---|---|---|---|---|---|---|
| | | LS Mean | | LS Mean | | Estimate | | |
| Test (N) | Ref (N) | (SE) | 95% CI | (SE) | 95% CI | (SE) | 95% CI | p-value |

Average Overall

| | | | | | | | | |
|---|---|---|---|---|---|---|---|---|
| HD | P | 0.04 | (−0.31, | 1.28 | (0.94, | −1.23 | (−1.72, | <.001*** |
| | | (0.177) | 0.40) | (0.166) | 1.61) | (0.243) | −0.75) | |
| LD | | 1.01 | (0.65, | | | −0.27 | (−0.75, | 0.271 |
| | | (0.177) | 1.36) | | | (0.243) | 0.22) | |
| M | | 0.79 | (0.44, | | | −0.49 | (−0.97, | 0.044* |
| | | (0.171) | 1.13) | | | (0.239) | −0.01) | |

2 HOURS POST INFUSION START

| | | | | | | | | |
|---|---|---|---|---|---|---|---|---|
| HD (18) | P (20) | −0.04 | (−0.51, | 1.15 | (0.70, | −1.19 | (−1.84, | <.001** |
| | | (0.238) | 0.44) | (0.225) | 1.60) | (0.327) | −0.53) | |
| LD (18) | | 0.13 | (−0.35, | | | −1.02 | (−1.67, | 0.003* |
| | | (0.238) | 0.61) | | | (0.327) | −0.36) | |
| M (19) | | 0.47 | (0.01, | | | −0.68 | (−1.32, | 0.039* |
| | | (0.231) | 0.93) | | | (0.322) | −0.04) | |

4 HOURS POST INFUSION START

| | | | | | | | | |
|---|---|---|---|---|---|---|---|---|
| HD (18) | P (20) | −0.04 | (−0.58, | 1.65 | (1.14, | −1.69 | (−2.44, | <.001*** |
| | | (0.273) | 0.51) | (0.258) | 2.16) | (0.375) | −0.94) | |
| LD (18) | | 0.52 | (−0.02, | | | −1.13 | (−1.88, | 0.004* |
| | | (0.273) | 1.06) | | | (0.375) | −0.38) | |
| M (19) | | 0.68 | (0.15, | | | −0.97 | (−1.71, | 0.011* |
| | | (0.265) | 1.21) | | | (0.370) | −0.23) | |

6 HOURS POST INFUSION START

| | | | | | | | | |
|---|---|---|---|---|---|---|---|---|
| HD (18) | P (20) | 0.07 | (−0.57, | 1.15 | (0.54, | −1.08 | (−1.97, | 0.018* |
| | | (0.324) | 0.72) | (0.307) | 1.76) | (0.446) | −0.19) | |
| LD (18) | | 1.80 | (1.15, | | | 0.65 | (−0.24, | 0.151 |
| | | (0.324) | 2.44) | | | (0.446) | 1.54) | |
| M (19) | | 0.84 | (0.21, | | | −0.31 | (−1.19, | 0.480 |
| | | (0.315) | 1.47) | | | (0.439) | 0.56) | |

8 HOURS POST INFUSION START

| | | | | | | | | |
|---|---|---|---|---|---|---|---|---|
| HD (18) | P (20) | 0.18 | (−0.37, | 1.15 | (0.63, | −0.97 | (−1.72, | 0.013* |
| | | (0.275) | 0.73) | (0.260) | 1.67) | (0.378) | −0.21) | |
| LD (18) | | 1.57 | (1.03, | | | 0.42 | (−0.33, | 0.265 |
| | | (0.275) | 2.12) | | | (0.378) | 1.18) | |
| M (19) | | 1.15 | (0.62, | | | 0.00 | (−0.74, | 0.992 |
| | | (0.267) | 1.69) | | | (0.372) | 0.75) | |

Note:
*,**,***indicate significance at 0.05, 0.001, and 0.0001 level, respectively.
Note:
MWT parameters were analyzed using a linear mixed effects model with fixed effects for sequence, period, treatment, time (as categorical variable) and treatment-by-time interaction, and a random effect for subject.
Note:
Stage N3 Duration and Stage REM Duration were not included in statistical analyses due to the lack of variability in measurements. LD: Compound A 44 mg; HD: Compound A 112 mg; M: Modafinil 300 mg; P: Placebo.
Program Source\T_BIOMWT2.sas (PRAHS SAS V9.4)

TABLE 13
Statistical Analysis of Additional MWT Endpoints
during Infusion - Stage N2 Duration
Pharmacodynamic Set
Parameter: Stage N2 Duration (min)

| | | Test | | Ref (Placebo) | | LS Mean Difference | | |
|---|---|---|---|---|---|---|---|---|
| | | LS Mean | | LS Mean | | Estimate | | |
| Test (N) | Ref (N) | (SE) | 95% CI | (SE) | 95% CI | (SE) | 95% CI | p-value |

Average Overall

| | | | | | | | | |
|---|---|---|---|---|---|---|---|---|
| HD | P | 0.09 | (−0.80, | 0.90 | (0.07, | −0.81 | (−2.03, | 0.184 |
| | | (0.441) | 0.97) | (0.418) | 1.73) | (0.608) | 0.40) | |
| LD | | 1.78 | (0.90, | | | 0.88 | (−0.33, | 0.153 |
| | | (0.441) | 2.66) | | | (0.608) | 2.09) | |
| M | | 0.41 | (−0.44, | | | −0.49 | (−1.68, | 0.419 |
| | | (0.429) | 1.27) | | | (0.599) | 0.71) | |

2 HOURS POST INFUSION START

| | | | | | | | | |
|---|---|---|---|---|---|---|---|---|
| HD (18) | P (20) | 0.00 | (−0.37, | 0.85 | (0.50, | −0.85 | (−1.36, | 0.001* |
| | | (0.186) | 0.37) | (0.175) | 1.20) | (0.255) | −0.34) | |
| LD (18) | | 0.24 | (−0.13, | | | −0.61 | (−1.12, | 0.019* |
| | | (0.186) | 0.61) | | | (0.255) | −0.10) | |
| M (19) | | 0.36 | (0.00, | | | −0.49 | (−0.99, | 0.055 |
| | | (0.180) | 0.72) | | | (0.251) | 0.01) | |

4 HOURS POST INFUSION START

| | | | | | | | | |
|---|---|---|---|---|---|---|---|---|
| HD (18) | P (20) | 0.00 | (−0.43, | 1.15 | (0.74, | −1.15 | (−1.74, | <.001** |
| | | (0.216) | 0.43) | (0.204) | 1.56) | (0.297) | −0.56) | |
| LD (18) | | 0.51 | (0.08, | | | −0.64 | (−1.23, | 0.036* |
| | | (0.216) | 0.95) | | | (0.297) | −0.04) | |
| M (19) | | 0.20 | (−0.21, | | | −0.95 | (−1.53, | 0.002* |
| | | (0.209) | 0.62) | | | (0.292) | −0.36) | |

6 HOURS POST INFUSION START

| | | | | | | | | |
|---|---|---|---|---|---|---|---|---|
| HD (18) | P (20) | 0.06 | (−1.39, | 0.50 | (−0.87, | −0.44 | (−2.44, | 0.659 |
| | | (0.726) | 1.50) | (0.688) | 1.87) | (1.000) | 1.55) | |
| LD (18) | | 2.63 | (1.18, | | | 2.13 | (0.13, | 0.037* |
| | | (0.726) | 4.07) | | | (1.000) | 4.12) | |
| M (19) | | 0.20 | (−1.21, | | | −0.30 | (−2.26, | 0.764 |
| | | (0.706) | 1.61) | | | (0.986) | 1.67) | |

8 HOURS POST INFUSION START

| | | | | | | | | |
|---|---|---|---|---|---|---|---|---|
| HD (18) | P (20) | 0.28 | (−1.70, | 1.10 | (−0.78, | −0.82 | (−3.55, | 0.550 |
| | | (0.992) | 2.26) | (0.941) | 2.98) | (1.367) | 1.91) | |
| LD (18) | | 3.74 | (1.76, | | | 2.64 | (−0.09, | 0.058 |
| | | (0.992) | 5.71) | | | (1.367) | 5.36) | |
| M (19) | | 0.89 | (−1.04, | | | −0.21 | (−2.90, | 0.875 |
| | | (0.965) | 2.81) | | | (1.348) | 2.47) | |

Note:
*,**,***indicate significance at 0.05, 0.001, and 0.0001 level, respectively.
Note:
MWT parameters were analyzed using a linear mixed effects model with fixed effects for sequence, period, treatment, time (as categorical variable) and treatment-by-time interaction, and a random effect for subject.
Note:
Stage N3 Duration and Stage REM Duration were not included in statistical analyses due to the lack of variability in measurements. LD: Compound A 44 mg; HD: Compound A 112 mg; M: Modafinil 300 mg; P: Placebo.
Program Source\T BIOMWT2.sas (PRAHS SAS V9 4)

TABLE 14
Statistical Analysis of Additional MWT Endpoints during
Infusion - Total Microsleeps
Pharmacodynamic Set
Parameter: Total Microsleeps

| | | Test | | Ref (Placebo) | | LS Mean Difference | | |
|---|---|---|---|---|---|---|---|---|
| | | LS Mean | | LS Mean | | Estimate | | |
| Test (N) | Ref (N) | (SE) | 95% CI | (SE) | 95% CI | (SE) | 95% CI | p-value |

Average Overall

| | | | | | | | | |
|---|---|---|---|---|---|---|---|---|
| HD | P | 0.31 | (−0.55, | 1.68 | (0.87, | ÷1.37 | (−2.55, | 0.023* |
| | | (0.429) | 1.16) | (0.404) | 2.48) | (0.590) | −0.19) | |
| LD | | 1.75 | (0.89, | | | 0.08 | (−1.10, | 0.899 |
| | | (0.429) | 2.61) | | | (0.590) | 1.25) | |
| M | | 1.60 | (0.77, | | | −0.08 | (−1.23, | 0.894 |
| | | (0.416) | 2.43) | | | (0.580) | 1.08) | |

2 HOURS POST INFUSION START

| | | | | | | | | |
|---|---|---|---|---|---|---|---|---|
| HD (18) | P (20) | 0.00 | (−0.87, | 1.80 | (0.98, | −1.80 | (−2.99, | 0.004* |
| | | (0.435) | 0.87) | (0.410) | 2.62) | (0.598) | −0.61) | |
| LD (18) | | 0.38 | (−0.49, | | | −1.42 | (−2.62, | 0.020* |
| | | (0.435) | 1.24) | | | (0.598) | −0.23) | |
| M (19) | | 1.06 | (0.22, | | | −0.74 | (−1.91, | 0.211 |
| | | (0.421) | 1.90) | | | (0.588) | 0.43) | |

4 HOURS POST INFUSION START

| | | | | | | | | |
|---|---|---|---|---|---|---|---|---|
| HD (18) | P (20) | 0.00 | (−1.40, | 2.35 | (1.02, | −2.35 | (−4.28, | 0.018* |
| | | (0.704) | 1.40) | (0.666) | 3.68) | (0.969) | −0.42) | |
| LD (18) | | 2.21 | (0.80, | | | −0.14 | (−2.07, | 0.884 |
| | | (0.704) | 3.61) | | | (0.969) | 1.79) | |
| M (19) | | 1.27 | (−0.10, | | | −1.08 | (−2.99, | 0.261 |
| | | (0.684) | 2.63) | | | (0.954) | 0.82) | |

6 HOURS POST INFUSION START

| | | | | | | | | |
|---|---|---|---|---|---|---|---|---|
| HD (18) | P (20) | 0.11 | (−1.08, | 1.25 | (0.13, | −1.14 | (−2.78, | 0.170 |
| | | (0.596) | 1.30) | (0.564) | 2.37) | (0.821) | 0.50) | |
| LD (18) | | 2.32 | (1.13, | | | 1.07 | (−0.57, | 0.197 |
| | | (0.596) | 3.51) | | | (0.821) | 2.71) | |
| M (19) | | 1.85 | (0.69, | | | 0.60 | (−1.01, | 0.462 |
| | | (0.579) | 3.00) | | | (0.808) | 2.21) | |

8 HOURS POST INFUSION START

| | | | | | | | | |
|---|---|---|---|---|---|---|---|---|
| HD (18) | P (20) | 1.11 | (−0.21, | 1.30 | (0.05, | −0.19 | (−2.00, | 0.836 |
| | | (0.661) | 2.43) | (0.625) | 2.55) | (0.910) | 1.63) | |
| LD (18) | | 2.10 | (0.78, | | | 0.80 | (−1.02, | 0.384 |
| | | (0.661) | 3.42) | | | (0.910) | 2.61) | |
| M (19) | | 2.22 | (0.94, | | | 0.92 | (−0.87, | 0.310 |
| | | (0.642) | 3.50) | | | (0.896) | 2.70) | |

Note:
*,**,***indicate significance at 0.05, 0.001, and 0.0001 level, respectively.
Note:
MWT parameters were analyzed using a linear mixed effects model with fixed effects for sequence, period, treatment, time (as categorical variable) and treatment-by-time interaction, and a random effect for subject.
Note:
Stage N3 Duration and Stage REM Duration were not included in statistical analyses due to the lack of variability in measurements. LD: Compound A 44 mg; HD: Compound A 112 mg; M: Modafinil 300 mg; P: Placebo.
Program Source\T_BIOMWT2.sas (PRAHS SAS V9.4)

TABLE 15
Statistical Analysis of Additional MWT Endpoints during
Infusion - Total Sleep Time
Pharmacodynamic Set
Parameter: Total Sleep Time (min)

| | | Test | | Ref (Placebo) | | LS Mean Difference | | |
|---|---|---|---|---|---|---|---|---|
| | | LS Mean | | LS Mean | | Estimate | | |
| Test (N) | Ref (N) | (SE) | 95% CI | (SE) | 95% CI | (SE) | 95% CI | p-value |

Average Overall

| | | | | | | | | |
|---|---|---|---|---|---|---|---|---|
| HD | P | 0.20 | (−0.81, | 2.76 | (1.80, | −2.56 | (−3.94, | <.001** |
| | | (0.505) | 1.20) | (0.477) | 3.71) | (0.694) | −1.17) | |
| LD | | 3.02 | (2.01, | | | 0.26 | (−1.13, | 0.709 |
| | | (0.505) | 4.02) | | | (0.694) | 1.65) | |
| M | | 1.36 | (0.38, | | | −1.40 | (−2.76, | 0.045* |
| | | (0.490) | 2.34) | | | (0.684) | −0.03) | |

2 HOURS POST INFUSION START

| | | | | | | | | |
|---|---|---|---|---|---|---|---|---|
| HD (18) | P (20) | 0.02 | (−0.75, | 2.73 | (2.00, | −2.71 | (−3.76, | <.001*** |
| | | (0.384) | 0.78) | (0.362) | 3.45) | (0.527) | −1.66) | |
| LD (18) | | 0.47 | (−0.30, | | | −2.26 | (−3.31, | <.001*** |
| | | (0.384) | 1.23) | | | (0.527) | −1.20) | |
| M (19) | | 0.88 | (0.14, | | | −1.85 | (−2.88, | <.001** |
| | | (0.372) | 1.62) | | | (0.519) | −0.81) | |

4 HOURS POST INFUSION START

| | | | | | | | | |
|---|---|---|---|---|---|---|---|---|
| HD (18) | P (20) | −0.01 | (−0.83, | 3.15 | (2.37, | −3.16 | (−4.29, | <.001*** |
| | | (0.413) | 0.81) | (0.389) | 3.93) | (0.567) | −2.03) | |
| LD (18) | | 1.27 | (0.45, | | | −1.88 | (−3.01, | 0.001* |
| | | (0.413) | 2.10) | | | (0.567) | −0.74) | |
| M (19) | | 0.96 | (0.16, | | | −2.19 | (−3.31, | <.001** |
| | | (0.400) | 1.75) | | | (0.558) | −1.08) | |

6 HOURS POST INFUSION START

| | | | | | | | | |
|---|---|---|---|---|---|---|---|---|
| HD (18) | P (20) | 0.21 | (−1.55, | 2.23 | (0.56, | −2.01 | (−4.44, | 0.103 |
| | | (0.884) | 1.97) | (0.837) | 3.89) | (1.217) | 0.41) | |
| LD (18) | | 4.69 | (2.93, | | | 2.47 | (0.04, | 0.047* |
| | | (0.884) | 6.45) | | | (1.217) | 4.89) | |
| M (19) | | 1.25 | (−0.47, | | | −0.98 | (−3.37, | 0.417 |
| | | (0.859) | 2.96) | | | (1.200) | 1.41) | |

8 HOURS POST INFUSION START

| | | | | | | | | |
|---|---|---|---|---|---|---|---|---|
| HD (18) | P (20) | 0.57 | (−1.53, | 2.93 | (0.93, | −2.35 | (−5.25, | 0.110 |
| | | (1.054) | 2.67) | (0.999) | 4.92) | (1.452) | 0.54) | |
| LD (18) | | 5.63 | (3.53, | | | 2.71 | (−0.19, | 0.066 |
| | | (1.054) | 7.74) | | | (1.452) | 5.61) | |
| M (19) | | 2.35 | (0.31, | | | −0.57 | (−3.43, | 0.690 |
| | | (1.025) | 4.40) | | | (1.432) | 2.28) | |

Note:
*,**,***indicate significance at 0.05, 0.001, and 0.0001 level, respectively.
Note:
MWT parameters were analyzed using a linear mixed effects model with fixed effects for sequence, period, treatment, time (as categorical variable) and treatment-by-time interaction, and a random effect for subject.
Note:
Stage N3 Duration and Stage REM Duration were not included in statistical analyses due to the lack of variability in measurements. LD: Compound A 44 mg; HD: Compound A 112 mg; M: Modafinil 300 mg; P: Placebo.
Program Source\T_BIOMWT2.sas (PRAHS SAS V9.4)

TABLE 16
Statistical Analysis of Additional MWT Endpoints during
Infusion - Total Wake Time
Pharmacodynamic Set
Parameter: Total Wake Time (min)

| | | Test | | Ref (Placebo) | | LS Mean Difference | | |
|---|---|---|---|---|---|---|---|---|
| | | LS Mean | | LS Mean | | Estimate | | |
| Test (N) | Ref (N) | (SE) | 95% CI | (SE) | 95% CI | (SE) | 95% CI | p-value |

Average Overall

| | | | | | | | | |
|---|---|---|---|---|---|---|---|---|
| HD | P | 39.05 | (35.13, | 9.46 | (5.78, | 29.59 | (24.21, | <..001*** |
| | | (1.965) | 42.97) | (1.845) | 13.15) | (2.696) | 34.97) | |
| LD | | 25.83 | (21.90, | | | 16.36 | (10.98, | <.001*** |
| | | (1.965) | 29.75) | | | (2.696) | 21.75) | |
| M | | 31.69 | (27.90, | | | 22.23 | (16.94, | <.001*** |
| | | (1.898) | 35.48) | | | (2.647) | 27.51) | |

2 HOURS POST INFUSION START

| | | | | | | | | |
|---|---|---|---|---|---|---|---|---|
| HD (18) | P (20) | 39.89 | (34.84, | 17.73 | (12.96, | 22.17 | (15.22, | <.001*** |
| | | (2.529) | 44.94) | (2.385) | 22.49) | (3.476) | 29.11) | |
| LD (18) | | 35.73 | (30.68, | | | 18.00 | (11.06, | <.001*** |
| | | (2.529) | 40.78) | | | (3.476) | 24.95) | |
| M (19) | | 34.87 | (29.98, | | | 17.15 | (10.32, | <.001*** |
| | | (2.450) | 39.77) | | | (3.419) | 23.98) | |

4 HOURS POST INFUSION START

| | | | | | | | | |
|---|---|---|---|---|---|---|---|---|
| HD (18) | P (20) | 40.08 | (35.41, | 10.05 | (5.65, | 30.03 | (23.62, | <.001*** |
| | | (2.340) | 44.76) | (2.205) | 14.45) | (3.215) | 36.45) | |
| LD (18) | | 32.42 | (27.75, | | | 22.37 | (15.96, | <.001*** |
| | | (2.340) | 37.09) | | | (3.215) | 28.79) | |
| M (19) | | 36.45 | (31.93, | | | 26.40 | (20.09, | <.001*** |
| | | (2.265) | 40.97) | | | (3.161) | 32.71) | |

6 HOURS POST INFUSION START

| | | | | | | | | |
|---|---|---|---|---|---|---|---|---|
| HD (18) | P (20) | 38.86 | (33.45, | 6.43 | (1.32, | 32.44 | (25.00, | <.001*** |
| | | (2.712) | 44.27) | (2.559) | 11.53) | (3.729) | 39.88) | |
| LD (18) | | 21.78 | (16.37, | | | 15.36 | (7.92, | <.001** |
| | | (2.712) | 27.19) | | | (3.729) | 22.80) | |
| M (19) | | 33.11 | (27.87, | | | 26.69 | (19.37, | <.001*** |
| | | (2.629) | 38.36) | | | (3.669) | 34.01) | |

8 HOURS POST INFUSION START

| | | | | | | | | |
|---|---|---|---|---|---|---|---|---|
| HD (18) | P (20) | 37.36 | (32.71, | 3.65 | (-0.74, | 33.71 | (27.31, | <.001*** |
| | | (2.332) | 42.02) | (2.197) | 8.04) | (3.204) | 40.11) | |
| LD (18) | | 13.37 | (8.71, | | | 9.72 | (3.32, | 0.003* |
| | | (2.332) | 18.02) | | | (3.204) | 16.12) | |
| M (19) | | 22.32 | (17.81, | | | 18.67 | (12.38, | <.001*** |
| | | (2.257) | 26.83) | | | (3.150) | 24.96) | |

Note:
*,**,***indicate significance at 0.05, 0.001, and 0.0001 level, respectively.
Note:
MWT parameters were analyzed using a linear mixed effects model with fixed effects for sequence, period, treatment, time (as categorical variable) and treatment-by-time interaction, and a random effect for subject.
Note:
Stage N3 Duration and Stage REM Duration were not included in statistical analyses due to the lack of variability in measurements. LD: Compound A 44 mg; HD: Compound A 112 mg; M: Modafinil 300 mg; P: Placebo.
Program Source\T_BIOMWT2.sas (PRAHS SAS V9.4)

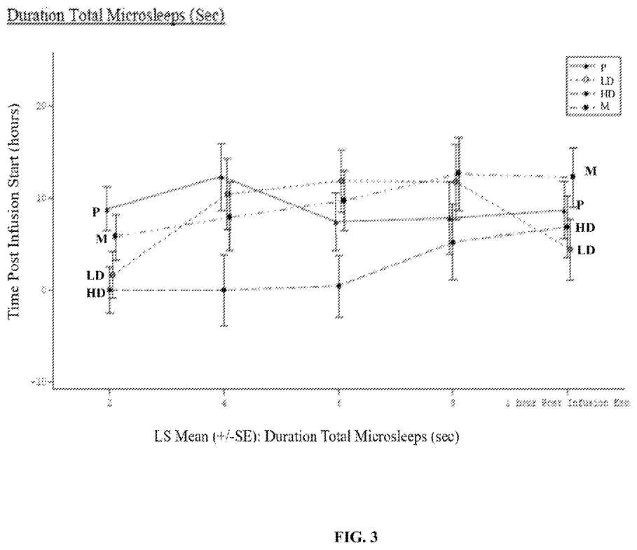

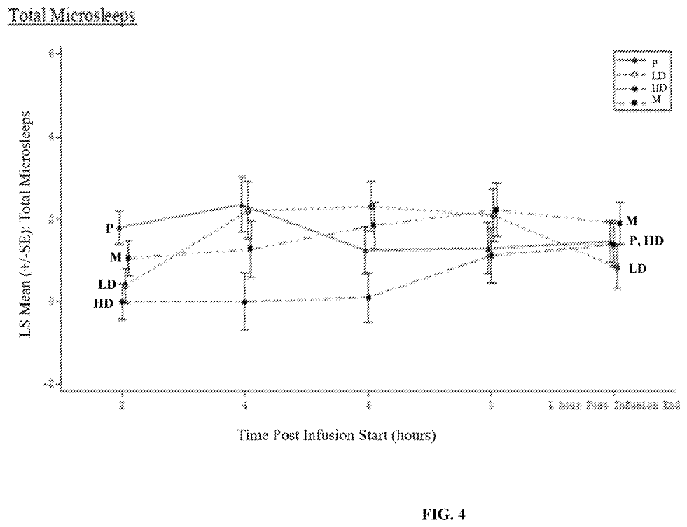

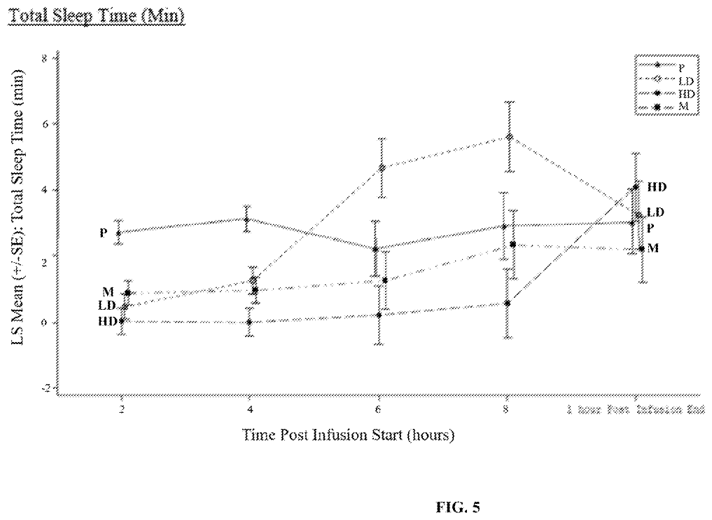

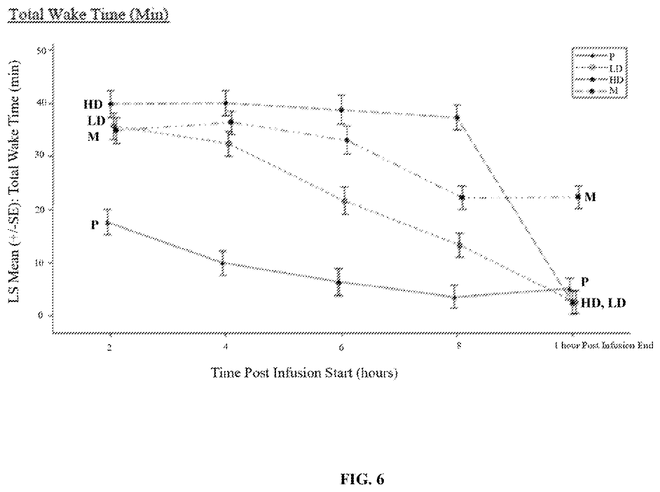

Plots of the LS mean (±SE) of additional MWT endpoints are presented in FIGS. 3-6. FIG. 3 shows LS Mean (±SE) of Duration of Total Microsleeps (Sec) versus time. FIG. 4 shows LS Mean (±SE) of Total Microsleeps versus time. FIG. 5 shows LS Mean (±SE) of Total Sleep Time (Min) versus time. FIG. 6 shows LS Mean (±SE) of Total Wake Time (Min) versus time.

The average total wake time results parallel the latency to sleep results on the MWT. For all 3 active treatments, the average total wake time postdose was significantly longer than that of the placebo. For Compound A 44 mg, the LS mean difference in average total wake time was 16.36 min (95% CI 10.98, 21.75). For the Compound A 112 mg, the LS mean difference in average total wake time was 29.59 min (95% CI 24.21, 34.97). For the reference compound, modafinil 300 mg, the LS mean difference in average total wake time was 22.23 min (95% CI 16.94, 27.51).

Total sleep time in the Compound A 44 mg was not significantly different than placebo. For Compound A 112 mg, total sleep time was statistically significantly shorter when compared to placebo (LS mean difference −2.56 min [95% CI −3.94, −1.17]). For modafinil 300 mg, total sleep time was also statistically significantly shorter when compared to placebo (LS mean difference −1.40 min [95% CI −2.76, −0.03]).

The average overall of total microsleep duration was not different for Compound A 44 mg and modafinil 300 mg than placebo; however, Compound A 112 mg did show a statistically significant difference when compared to placebo for both (LS mean difference −7.68 sec [95% CI −14.5, −0.89] microsleep duration and total number of microsleeps (LS mean difference −1.37 [95% CI −2.55, −0.19]).

Drug Dose, Drug Concentration, and Relationships to Response

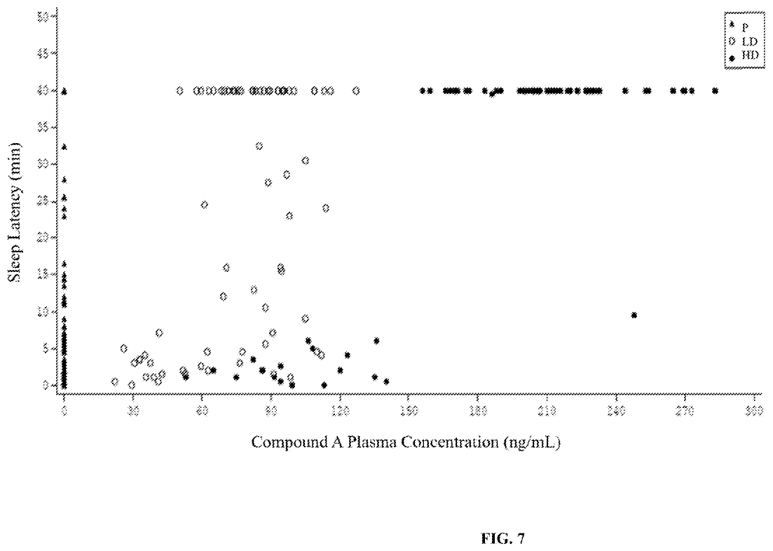

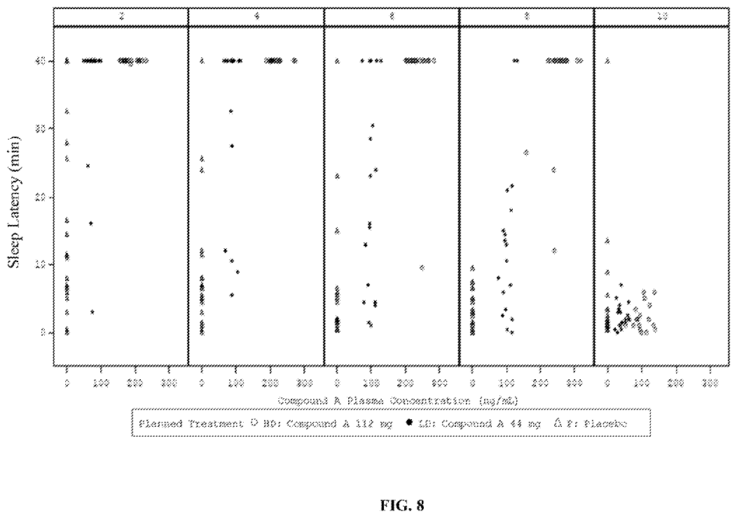

Consistent with the observations that most subjects receiving Compound A 112 mg stayed awake throughout the MWT sessions while subjects receiving Compound A 44 mg progressively tended to fall asleep, FIG. 7 showed that higher plasma concentrations of Compound A generally resulted in greater maintenance of wakefulness. However, Compound A wake-promoting effects were noticeably variable across the concentration range associated with the lowest dose of 44 mg, whereas a steep concentration-effect relationship was observed at the highest dose of 112 mg. It can be seen from plotting latency in sleep onset versus Compound A concentration by scheduled MWT session that the concentration-effect relationship is time-dependent in FIG. 8. The magnitude of the wake-promoting effects appeared to diminish over time at concentrations of Compound A less than 200 ng/mL, while wakefulness could be maintained with higher Compound A concentrations up to the last MWT session (8-hour after start of infusion). At the 10-hour timepoint however, which was one hour after the end of the Compound A infusion, the ability to remain awake declined to that of placebo due to low residual drug concentrations.

### Example I-5: Sleepiness/Alertness as Assessed by Karolinska Sleepiness Scale (KSS)

Secondary Efficacy Endpoint—KSS

The secondary efficacy endpoint was sleepiness/alertness as assessed by KSS at 14, 10, 6, and 2 hours predose (approximately between 7:45 AM to 7:45 PM); 2, 2.75, 4.75, 6.75, and 8.75 hours post infusion start (approximately between mid-night to 7 AM in the morning); and at 1.75 hours post infusion end. A higher value indicates more sleepiness, a lower value indicates more alertness. Prior to infusion, there were no statistically significant differences at any time point of observation in KSS score for Compound A 44 and 112 mg treatments when compared to placebo. After the start of infusion, statistically significant differences in KSS score were observed at all time points during the infusion for Compound A 112 mg and modafinil 300 mg; however, Compound A 44 mg treatment was statistically significantly different only at 2.75 and 4.75 hours post infusion start. On the average, for modafinil and Compound A 44 and 112 mg, there were about 1.6 to 3.7 points increase in the KSS postdose/the infusion start, relative to that prior to dosing during the daytime. However, for placebo, the increase in the average KSS is 4.9. The placebo-adjusted LS mean (95% CI) of change in KSS scores from prior to infusion to post were −2.53 (−3.43, −1.63), −1.22 (−2.13, −0.301) and −3.26 (−4.17, −2.34) for modafinil 300 mg, Compound A 44 and 112 mg, respectively, which was significantly different than the placebo change in the KSS.

After infusion end (1.75 hours post infusion end), only modafinil 300 mg has a KSS score lower than placebo (7.16 vs. 8.15, p<0.05).

Summary of sleepy/alert as assessed by KSS at 14, 10, 6, and 2 hours predose; 2, 2.75, 4.75, 6.75, and 8.75 hours post infusion start; and at 1.75 hours post infusion end is presented in Tables 17 and 18. Statistical analysis of sleepiness as assessed by KSS is presented in Tables 19-21.

Table 22 shows the difference between average KSS score post and prior to infusion start.

TABLE 17
Summary of Sleepiness assessed by the Karolinska Sleepiness Scale (KSS)
Pharmacodynamic Set

| | | | Compound A | Compound A | Modafinil |
|---|---|---|---|---|---|
| | | Placebo | 44 mg | 112 mg | 300 mg |
| Time Point | Statistic | (N = 20) | (N = 18) | (N = 18) | (N = 19) |
| 14 HOURS PREDOSE | N | 20 | 18 | 18 | 19 |
| | Mean (SD) | 3.1 (1.43) | 2.8 (1.15) | 3.3 (1.74) | 2.8 (1.08) |
| | Median | 3.0 | 3.0 | 3.0 | 3.0 |
| | Min, Max | 1, 6 | 1, 6 | 1, 7 | 1, 5 |
| 10 HOURS PREDOSE | N | 20 | 18 | 18 | 19 |
| | Mean (SD) | 2.5 (1.10) | 2.8 (1.40) | 2.5 (1.47) | 2.3 (1.11) |
| | Median | 2.5 | 3.0 | 2.0 | 2.0 |
| | Min, Max | 1, 5 | 1, 7 | 1, 7 | 1, 5 |
| 6 HOURS PREDOSE | N | 20 | 18 | 18 | 19 |
| | Mean (SD) | 2.8 (1.32) | 2.8 (1.59) | 2.5 (0.92) | 2.6 (1.35) |
| | Median | 2.5 | 2.5 | 3.0 | 2.0 |
| | Min, Max | 1, 5 | 1, 7 | 1, 4 | 1, 7 |
| 2 HOURS PREDOSE | N | 20 | 18 | 18 | 19 |
| | Mean (SD) | 3.9 (2.02) | 4.0 (2.30) | 3.4 (1.76) | 3.3 (1.20) |
| | Median | 3.5 | 3.5 | 3.0 | 3.0 |
| | Min, Max | 1, 8 | 1, 9 | 1, 6 | 1, 6 |
| 2.75 HOURS POST | N | 20 | 18 | 18 | 19 |
| INFUSION START | Mean (SD) | 6.9 (1.81) | 4.8 (1.92) | 3.2 (2.02) | 3.7 (1.63) |
| | Median | 7.0 | 4.5 | 2.0 | 4.0 |
| | Min, Max | 3, 9 | 1, 8 | 1, 8 | 1, 6 |

LD: Compound A 44 mg; HD: Compound A 112 mg; M: Modafinil 300 mg; P: Placebo.
Note:
KSS is 9-item Likert-type rating scale for assessing subjective sleepiness: 1 = very alert, 3 = alert, 5 = neither alert nor sleepy, 7 = sleepy (but not fighting sleep), 9 = very sleepy (fighting sleep)
Program Source\T_KSS.sas (PRAHS SAS V9.4)

TABLE 18
Summary of Sleepiness assessed by the Karolinska Sleepiness Scale (KSS)
Pharmacodynamic Set

| | | | Compound A | Compound A | Modafinil |
|---|---|---|---|---|---|
| | | Placebo | 44 mg | 112 mg | 300 mg |
| Time Point | Statistic | (N = 20) | (N = 18) | (N = 18) | (N = 19) |
| 4.75 HOURS POST | N | 20 | 18 | 18 | 19 |
| INFUSION START | Mean (SD) | 8.2 (0.99) | 6.4 (2.12) | 3.8 (1.82) | 4.3 (1.89) |
| | Median | 8.5 | 6.5 | 3.0 | 4.0 |
| | Min, Max | 6, 9 | 2, 9 | 1, 9 | 1, 7 |
| 6.75 HOURS POST | N | 20 | 18 | 18 | 19 |
| INFUSION START | Mean (SD) | 8.5 (0.69) | 7.8 (0.94) | 5.2 (1.72) | 5.8 (2.12) |
| | Median | 9.0 | 8.0 | 5.0 | 6.0 |
| | Min, Max | 7, 9 | 6, 9 | 2, 9 | 2, 8 |
| 8.75 HOURS POST | N | 19 | 18 | 18 | 19 |
| INFUSION START | Mean (SD) | 8.3 (0.95) | 8.1 (1.00) | 6.1 (1.95) | 6.7 (1.92) |
| | Median | 9.0 | 8.0 | 6.0 | 7.0 |
| | Min, Max | 6, 9 | 5, 9 | 3, 9 | 3, 9 |
| 1.75 HOURS POST | N | 20 | 18 | 18 | 19 |
| INFUSION END | Mean (SD) | 8.2 (0.88) | 8.4 (0.86) | 8.0 (1.57) | 7.2 (1.80) |
| | Median | 8.0 | 9.0 | 8.5 | 8.0 |
| | Min, Max | 6, 9 | 6, 9 | 3, 9 | 3, 9 |

LD: Compound A 44 mg; HD: Compound A 112 mg; M: Modafinil 300 mg; P: Placebo.
Note:
KSS is 9-item Likert-type rating scale for assessing subjective sleepiness: 1 = very alert, 3 = alert, 5 = neither alert nor sleepy, 7 = sleepy (but not fighting sleep), 9 = very sleepy (fighting sleep)
Program Source\T_KSS.sas (PRAHS SAS V9.4)

TABLE 19
Summary of Sleepiness assessed by the Karolinska Sleepiness
Scale (KSS) - Sleepy/Alert
Pharmacodynamic Set
Parameter: Sleepy/Alert

| | | Test | | Ref (Placebo) | | LS Mean Difference | | |
|---|---|---|---|---|---|---|---|---|
| | | LS Mean | | LS Mean | | Estimate | | |
| Test (N) | Ref(N) | (SE) | 95% CI | (SE) | 95% CI | (SE) | 95% CI | p-value |

14 HOURS PREDOSE

| | | | | | | | | |
|---|---|---|---|---|---|---|---|---|
| HD (18) | P (20) | 3.29 | (2.62, | 3.05 | (2.41, | 0.24 | (−0.69, | 0.605 |
| | | (0.339) | 3.97) | (0.320) | 3.69) | (0.466) | 1.17) | |
| LD (18) | | 2.85 | (2.17, | | | −0.20 | (−1.13, | 0.664 |
| | | (0.339) | 3.52) | | | (0.466) | 0.73) | |
| M (19) | | 2.79 | (2.14, | | | −0.26 | (−1.17, | 0.574 |
| | | (0.328) | 3.45) | | | (0.458) | 0.66) | |

10 HOURS PREDOSE

| | | | | | | | | |
|---|---|---|---|---|---|---|---|---|
| HD (18) | P (20) | 2.51 | (1.92, | 2.50 | (1.94, | 0.01 | (−0.81, | 0.972 |
| | | (0.300) | 3.11) | (0.283) | 3.06) | (0.412) | 0.84) | |
| LD (18) | | 2.79 | (2.19, | | | 0.29 | (−0.53, | 0.483 |
| | | (0.300) | 3.39) | | | (0.412) | 1.11) | |
| M (19) | | 2.32 | (1.74, | | | −0.18 | (−0.99, | 0.654 |
| | | (0.291) | 2.90) | | | (0.406) | 0.63) | |

6 HOURS PREDOSE

| | | | | | | | | |
|---|---|---|---|---|---|---|---|---|
| HD (18) | P (20) | 2.51 | (1.89, | 2.80 | (2.21, | −0.29 | (−1.14, | 0.508 |
| | | (0.312) | 3.14) | (0.294) | 3.39) | (0.429) | 0.57) | |
| LD (18) | | 2.79 | (2.17, | | | −0.01 | (−0.86, | 0.984 |
| | | (0.312) | 3.41) | | | (0.429) | 0.85) | |
| M (19) | | 2.58 | (1.98, | | | −0.22 | (−1.06, | 0.605 |
| | | (0.302) | 3.18) | | | (0.422) | 0.62) | |

2 HOURS PREDOSE

| | | | | | | | | |
|---|---|---|---|---|---|---|---|---|
| HD (18) | P (20) | 3.46 | (2.61, | 3.90 | (3.09, | −0.44 | (−1.61, | 0.455 |
| | | (0.426) | 4.31) | (0.403) | 4.71) | (0.587) | 0.73) | |
| LD (18) | | 4.01 | (3.16, | | | 0.11 | (−1.06, | 0.847 |
| | | (0.426) | 4.87) | | | (0.587) | 1.29) | |
| M (19) | | 3.32 | (2.49, | | | −0.58 | (−1.74, | 0.317 |
| | | (0.414) | 4.14) | | | (0.578) | 0.57) | |

Note:
*,**,***indicate significance at 0.05, 0.001, and 0.0001 level, respectively.
Note:
KSS is 9-item Likert-type rating scale for assessing subjective sleepiness: 1 = very alert, 3 = alert, 5 = neither alert nor sleepy, 7 = sleepy (but not fighting sleep), 9 = very sleepy (fighting sleep)
Note:
Sleepiness was analyzed using a linear mixed effects model with fixed effects for sequence, period, treatment, time (as categorical variable) and treatment-by-time interaction, and a random effect for subject.
LD: Compound A 44 mg; HD: Compound A 112 mg; M: Modafinil 300 mg; P: Placebo.
Program Source\T_BIOKSS.sas (PRAHS SAS V9.4)

TABLE 20
Summary of Sleepiness assessed by the Karolinska Sleepiness
Scale (KSS) - Sleepy/Alert
Pharmacodynamic Set
Parameter: Sleepy/Alert

| | | Test | | Ref (Placebo) | | LS Mean Difference | | |
|---|---|---|---|---|---|---|---|---|
| | | LS Mean | | LS Mean | | Estimate | | |
| Test (N) | Ref (N) | (SE) | 95% CI | (SE) | 95% CI | (SE) | 95% CI | p-value |

2.75 HOURS POST INFUSION START

| | | | | | | | | |
|---|---|---|---|---|---|---|---|---|
| HD (18) | P (20) | 3.24 | (2.38, | 6.85 | (6.04, | −3.61 | (−4.79, | <.001*** |
| | | (0.429) | 4.09) | (0.405) | 7.66) | (0.590) | −2.44) | |
| LD (18) | | 4.85 | (3.99, | | | −2.00 | (−3.18, | 0.001* |
| | | (0.429) | 5.70) | | | (0.590) | −0.83) | |
| M (19) | | 3.69 | (2.86, | | | −3.16 | (−4.32, | <.001*** |
| | | (0.416) | 4.52) | | | (0.581) | −2.01) | |

4.75 HOURS POST INFUSION START

| | | | | | | | | |
|---|---|---|---|---|---|---|---|---|
| HD (18) | P (20) | 3.85 | (3.05, | 8.15 | (7.40, | −4.30 | (−5.39, | <.001*** |
| | | (0.397) | 4.64) | (0.376) | 8.90) | (0.547) | −3.21) | |
| LD (18) | | 6.46 | (5.66, | | | −1.69 | (−2.78, | 0.003* |
| | | (0.397) | 7.25) | | | (0.547) | −0.60) | |
| M (19) | | 4.32 | (3.55, | | | −3.83 | (−4.91, | <.001*** |
| | | (0.386) | 5.09) | | | (0.538) | −2.76) | |

6.75 HOURS POST INFUSION START

| | | | | | | | | |
|---|---|---|---|---|---|---|---|---|
| HD (18) | P (20) | 5.18 | (4.51, | 8.50 | (7.87, | −3.32 | (−4.24, | <.001*** |
| | | (0.336) | 5.85) | (0.317) | 9.13) | (0.462) | −2.40) | |
| LD (18) | | 7.79 | (7.12, | | | −0.71 | (−1.63, | 0.130 |
| | | (0.336) | 8.46) | | | (0.462) | 0.21) | |
| M (19) | | 5.79 | (5.14, | | | −2.71 | (−3.62, | <.001*** |
| | | (0.326) | 6.44) | | | (0.455) | −1.80) | |

8.75 HOURS POST INFUSION START

| | | | | | | | | |
|---|---|---|---|---|---|---|---|---|
| HD (18) | P (19) | 6.07 | (5.39, | 8.33 | (7.68, | −2.26 | (−3.20, | <.001*** |
| | | (0.340) | 6.75) | (0.325) | 8.98) | (0.471) | −1.32) | |
| LD (18) | | 8.07 | (7.39, | | | −0.26 | (−1.20, | 0.577 |
| | | (0.340) | 8.75) | | | (0.471) | 0.68) | |
| M (19) | | 6.69 | (6.03, | | | −1.65 | (−2.57, | <.001** |
| | | (0.330) | 7.35) | | | (0.464) | −0.72) | |

1.75 HOURS POST INFUSION END

| | | | | | | | | |
|---|---|---|---|---|---|---|---|---|
| HD (18) | P (20) | 8.01 | (7.39, | 8.15 | (7.56, | −0.14 | (−0.99, | 0.753 |
| | | (0.312) | 8.64) | (0.294) | 8.74) | (0.429) | 0.72) | |
| LD (18) | | 8.46 | (7.84, | | | 0.31 | (−0.55, | 0.475 |
| | | (0.312) | 9.08) | | | (0.429) | 1.16) | |
| M (19) | | 7.16 | (6.56, | | | −0.99 | (−1.83, | 0.022* |
| | | (0.302) | 7.76) | | | (0.422) | −0.15) | |

Note:
*,**,***indicate significance at 0.05, 0.001, and 0.0001 level, respectively.
Note:
KSS is 9-item Likert-type rating scale for assessing subjective sleepiness: 1 = very alert, 3 = alert, 5 = neither alert nor sleepy, 7 = sleepy (but not fighting sleep), 9 = very sleepy (fighting sleep)
Note:
Sleepiness was analyzed using a linear mixed effects model with fixed effects for sequence, period, treatment, time (as categorical variable) and treatment-by-time interaction, and a random effect for subject.
LD: Compound A 44 mg; HD: Compound A 112 mg; M: Modafinil 300 mg; P: Placebo.
Program Source\T_BIOKSS.sas (PRAHS SAS V9.4)

TABLE 21
Summary of Sleepiness assessed by the Karolinska
Sleepiness Scale (KSS) - Sleepy/Alert
Pharmacodynamic Set
Parameter: Sleepy/Alert

| | | Test | | Ref (Placebo) | | LS Mean Difference | | |
|---|---|---|---|---|---|---|---|---|
| | | LS Mean | | LS Mean | | Estimate | | |
| Test (N) | Ref (N) | (SE) | 95% CI | (SE) | 95% CI | (SE) | 95% CI | p-value |

AVERAGE PRIOR TO INFUSION START (14, 10, 6, 2 HOURS PREDOSE)

| | | | | | |
|---|---|---|---|---|---|
| HD | P | 2.95 | (2.39, | 3.06 | (2.54, |
| | | (0.277) | 3.50) | (0.261) | 3.58) |
| LD | | 3.11 | (2.56, | | |
| | | (0.277) | 3.66) | | |
| M | | 2.75 | (2.22, | | |
| | | (0.268) | 3.29) | | |

AVERAGE POST INFUSION START (2.75, 4.75, 6.75 AND 8.75 HOURS)

| | | | | | |
|---|---|---|---|---|---|
| HD | P | 4.58 | (4.01, | 7.96 | (7.42, |
| | | (0.286) | 5.15) | (0.269) | 8.50) |
| LD | | 6.79 | ( 6.22, | | |
| | | (0.286) | 7.36) | | |
| M | | 5.12 | (4.57, | | |
| | | (0.276) | 5.67) | | |

DELTA: AVERAGE POST - AVERAGE PRIOR

| | | | | | | | | |
|---|---|---|---|---|---|---|---|---|
| HD | P | 1.64 | (0.98, | 4.90 | (4.27, | −3.26 | (−4.17, | <.001*** |
| | | (0.332) | 2.30) | (0.315) | 5.52) | (0.458) | −2.34) | |
| LD | | 3.68 | (3.02, | | | −1.22 | (−2.13, | 0.010* |
| | | (0.332) | 4.34) | | | (0.458) | −0.30) | |
| M | | 2.37 | (1.72, | | | −2.53 | (−3.43, | <.001*** |
| | | (0.323) | 3.01) | | | (0.451) | −1.63) | |

Note:
*,**,***indicate significance at 0.05, 0.001, and 0.0001 level, respectively.
Note:
KSS is 9-item Likert-type rating scale for assessing subjective sleepiness: 1 = very alert, 3 = alert, 5 = neither alert nor sleepy, 7 = sleepy (but not fighting sleep), 9 = very sleepy (fighting sleep)
Note:
Sleepiness was analyzed using a linear mixed effects model with fixed effects for sequence, period, treatment, time (as categorical variable) and treatment-by-time interaction, and a random effect for subject.
LD: Compound A 44 mg; HD: Compound A 112 mg; M: Modafinil 300 mg; P: Placebo.
Program Source\T_BIOKSS.sas (PRAHS SAS V9.4)

TABLE 22
Difference Between Average KSS Score Post and Prior to Infusion Start

| | | Test | LS Mean Difference | | |
|---|---|---|---|---|---|
| Test | N | LS Mean (SE) | Estimate (SE) | 95% CI | p-value |
| Placebo | 20 | 4.90 (0.315) | | (4.27, 5.52) | |
| Compound A 44 mg | 18 | 3.68 (0.332) | −1.22 (0.458) | (−2.13. −0.30) | 0.010* |
| Compound A 112 mg | 18 | 1.64 (0.332) | −3.26 (0.458) | (−4.17, −2.34) | <0.001*** |
| Modafinil 300 mg | 19 | 2.37 (0.323) | −2.53 (0.451) | (−3.43. −1.63) | <0.001*** |

CI: confidence interval; KSS: Karolinska Sleepiness Scale; LS: least squares; SE: standard error.
*,***indicate significance at the 0.05 and 0.0001 level, respectively.

### Example I-6: Additional Exploratory PD Endpoints

PSG

The PSGs assess recovery sleep during the day after the end of the sleep deprivation period. Statistical analysis of PSG parameters is provided in Tables 23-29. There were no corrections for multiple comparisons. The PSGs assess recovery sleep during the day after the end of the sleep deprivation period.

TABLE 23
Statistical Analysis of PSG Parameters
Pharmacodynamic Set

| | | Test | | Ref (Placebo) | | LS Mean Difference | | |
|---|---|---|---|---|---|---|---|---|
| | | LS Mean | | LS Mean | | Estimate | | |
| Test (N) | Ref(N) | (SE) | 95% CI | (SE) | 95% CI | (SE) | 95% CI | p-value |

Awakenings

| | | | | | | | | |
|---|---|---|---|---|---|---|---|---|
| HD (18) | P (20) | 5.71 | (4.43, | 4.49 | (3.29, | 1.22 | (−0.34, | 0.122 |
| | | (0.635) | 6.98) | (0.598) | 5.69) | (0.774) | 2.77) | |
| LD (18) | | 4.74 | (3.47, | | | 0.25 | (−1.31, | 0.749 |
| | | (0.635) | 6.01) | | | (0.774) | 1.80) | |
| M (19) | | 5.38 | (4.15, | | | 0.89 | (−0.63, | 0.245 |
| | | (0.614) | 6.61) | | | (0.757) | 2.41) | |

N1 Latency (min)

| | | | | | | | | |
|---|---|---|---|---|---|---|---|---|
| HD (18) | P (20) | 2.89 | (0.59, | 5.37 | (3.21, | −2.47 | (−5.53, | 0.11 |
| | | (1.151) | 5.19) | (1.078) | 7.52) | (1.521) | 0.58) | |
| LD (18) | | 3.60 | (1.30, | | | −1.76 | (−4.82, | 0.252 |
| | | (1.151) | 5.90) | | | (1.521) | 1.29) | |
| M (19) | | 6.07 | (3.85, | | | 0.71 | (−2.28, | 0.637 |
| | | (1.110) | 8.29) | | | (1.490) | 3.70) | |

N2 Latency (min)

| | | | | | | | | |
|---|---|---|---|---|---|---|---|---|
| HD (18) | P (20) | 4.72 | (2.90, | 6.64 | (4.94, | −1.93 | (−4.28, | 0.107 |
| | | (0.909) | 6.53) | (0.852) | 8.35) | (1.172) | 0.43) | |
| LD (18) | | 3.91 | (2.09, | | | −2.74 | (−5.09, | 0.024* |
| | | (0.909) | 5.72) | | | (1.172) | −0.38) | |
| M (19) | | 8.62 | (6.87, | | | 1.98 | (−0.33, | 0.091 |
| | | (0.877) | 10.38) | | | (1.148) | 4.29) | |

N3 Latency (min)

| | | | | | | | | |
|---|---|---|---|---|---|---|---|---|
| HD (18) | P (20) | 15.83 | (5.63, | 27.27 | (17.72, | −11.45 | (−24.87, | 0.093 |
| | | (5.098) | 26.02) | (4.779) | 36.83) | (6.689) | 1.98) | |
| LD (18) | | 21.94 | (11.75, | | | −5.33 | (−18.75, | 0.429 |
| | | (5.098) | 32.14) | | | (6.689) | 8.09) | |
| M (19) | | 38.00 | (28.17, | | | 10.73 | (−2.43, | 0.108 |
| | | (4.917) | 47.83) | | | (6.553) | 23.88) | |

Per. Slp (min)

| | | | | | | | | |
|---|---|---|---|---|---|---|---|---|
| HD (18) | P (20) | 4.50 | (−4.39, | 6.87 | (−1.45, | −2.37 | (−14.60, | 0.699 |
| | | (4.450) | 13.39) | (4.169) | 15.20) | (6.094) | 9.86) | |
| LD (18) | | 5.96 | (−2.93, | | | −0.91 | (−13.14, | 0.882 |
| | | (4.450) | 14.85) | | | (6.094) | 11.32) | |
| M (19) | | 26.19 | (17.62, | | | 19.32 | (7.32, | 0.002* |
| | | (4.290) | 34.77) | | | (5.980) | 31.32) | |

Note:
*,**,***indicate significance at 0.05, 0.001, and 0.0001 level, respectively.
Note:
PSG parameters were analyzed using a linear mixed effects model with fixed effects for sequence, period, and treatment, and a random effect for subject nested in sequence. Baseline PSG was included as a fixed-effect continuous variable in the model.
LD: Compound A 44 mg; HD: Compound A 112 mg; M: Modafinil 300 mg; P: Placebo.
Program Source\T_BIOPSG.sas (PRAHS SAS V9.4)

TABLE 24
Statistical Analysis of PSG Parameters
Pharmacodynamic Set

| | | Test | | Ref (Placebo) | | LS Mean Difference | | |
|---|---|---|---|---|---|---|---|---|
| | | LS Mean | | LS Mean | | Estimate | | |
| Test (N) | Ref (N) | (SE) | 95% CI | (SE) | 95% CI | (SE) | 95% CI | p-value |

REM Latency (min)

| | | | | | | | | |
|---|---|---|---|---|---|---|---|---|
| HD (18) | P (20) | 84.57 | (62.59, | 54.93 | (34.02, | 29.64 | (5.44, | 0.017* |
| | | (10.91) | 106.56) | (10.35) | 75.85) | (12.05) | 53.84) | |
| LD (18) | | 75.10 | (53.11, | | | 20.16 | (−4.03, | 0.100 |
| | | (10.91) | 97.08) | | | (12.05) | 44.36) | |
| M (19) | | 71.67 | (50.25, | | | 16.73 | (−6.92, | 0.162 |
| | | (10.61) | 93.08) | | | (11.77) | 40.38) | |

Sleep Efficiency (%)

| | | | | | | | | |
|---|---|---|---|---|---|---|---|---|
| HD (18) | P (20) | 84.65 | (78.91, | 86.72 | (81.30, | −2.06 | (−8.93, | 0.549 |
| | | (2.863) | 90.40) | (2.698) | 92.14) | (3.421) | 4.81) | |
| LD (18) | | 84.95 | (79.21, | | | −1.77 | (−8.64, | 0.608 |
| | | (2.863) | 90.70) | | | (3.421) | 5.10) | |
| M (19) | | 68.28 | (62.71, | | | −18.44 | (−25.16, | <.001*** |
| | | (2.773) | 73.85) | | | (3.347) | −11.72) | |

Sleep Onset (min)

| | | | | | | | | |
|---|---|---|---|---|---|---|---|---|
| HD (18) | P (20) | 2.94 | (1.29, | 3.99 | (2.44, | −1.04 | (−3.21, | 0.339 |
| | | (0.826) | 4.60) | (0.774) | 5.54) | (1.081) | 1.13) | |
| LD (18) | | 2.71 | (1.05, | | | −1.28 | (−3.45, | 0.241 |
| | | (0.826) | 4.36) | | | (1.081) | 0.89) | |
| M (19) | | 5.99 | (4.40, | | | 2.00 | (−0.12, | 0.064 |
| | | (0.797) | 7.59) | | | (1.058) | 4.13) | |

Spontaneous Arousal

| | | | | | | | | |
|---|---|---|---|---|---|---|---|---|
| HD (18) | P (20) | 41.46 | (33.50, | 45.07 | (37.54, | −3.61 | (−12.85, | 0.437 |
| | | (3.963) | 49.42) | (3.745) | 52.61) | (4.604) | 5.63) | |
| LD (18) | | 40.21 | (32.25, | | | −4.86 | (−14.10, | 0.296 |
| | | (3.963) | 48.17) | | | (4.604) | 4.38) | |
| M (19) | | 42.14 | (34.41, | | | −2.93 | (−11.97, | 0.518 |
| | | (3.847) | 49.88) | | | (4.502) | 6.11) | |

Spontaneous Arousal Index

| | | | | | | | | |
|---|---|---|---|---|---|---|---|---|
| HD (18) | P (20) | 8.26 | (6.46, | 8.93 | (7.23, | −0.67 | (−2.93, | 0.553 |
| | | (0.902) | 10.07) | (0.848) | 10.63) | (1.124) | 1.58) | |
| LD (18) | | 8.58 | (6.77, | | | −0.36 | (−2.61, | 0.751 |
| | | (0.902) | 10.38) | | | (1.124) | 1.90) | |
| M (19) | | 11.91 | (10.16, | | | 2.98 | (0.77, | 0.009* |
| | | (0.873) | 13.66) | | | (1.100) | 5.19) | |

Note:
*,**,***indicate significance at 0.05, 0.001, and 0.0001 level, respectively.
Note:
PSG parameters were analyzed using a linear mixed effects model with fixed effects for sequence, period, and treatment, and a random effect for subject nested in sequence. Baseline PSG was included as a fixed-effect continuous variable in the model.
LD: Compound A 44 mg; HD: Compound A 112 mg; M: Modafinil 300 mg; P: Placebo.
Program Source\T_BIOPSG.sas (PRAHS SAS V9.4)

TABLE 25
Statistical Analysis of PSG Parameters
Pharmacodynamic Set

| | | Test | | Ref (Placebo) | | LS Mean Difference | | |
|---|---|---|---|---|---|---|---|---|
| | | LS Mean | | LS Mean | | Estimate | | |
| Test (N) | Ref (N) | (SE) | 95% CI | (SE) | 95% CI | (SE) | 95% CI | p-value |

Stage N1 %

| | | | | | | | | |
|---|---|---|---|---|---|---|---|---|
| HD (18) | P (20) | 6.08 | (3.91, | 6.08 | (4.02, | 0.00 | (−2.52, | 1.000 |
| | | (1.083) | 8.26) | (1.023) | 8.14) | (1.257) | 2.52) | |
| LD (18) | | 5.69 | (3.52, | | | −0.39 | (−2.91, | 0.759 |
| | | (1.083) | 7.87) | | | (1.257) | 2.13) | |
| M (19) | | 10.14 | (8.02, | | | 4.05 | (1.58, | 0.002* |
| | | (1.051) | 12.25) | | | (1.229) | 6.52) | |

Stage N1 Duration (min)

| | | | | | | | | |
|---|---|---|---|---|---|---|---|---|
| HD (18) | P (20) | 18.05 | (13.75, | 18.41 | (14.28, | −0.35 | (−4.68, | 0.870 |
| | | (2.126) | 22.36) | (2.030) | 22.53) | (2.156) | 3.98) | |
| LD (18) | | 16.29 | (11.98, | | | −2.12 | (−6.45, | 0.330 |
| | | (2.126) | 20.59) | | | (2.156) | 2.21) | |
| M (19) | | 19.12 | (14.91, | | | 0.72 | (−3.51, | 0.735 |
| | | (2.076) | 23.34) | | | (2.107) | 4.95) | |

Stage N2 %

| | | | | | | | | |
|---|---|---|---|---|---|---|---|---|
| HD (18) | P (20) | 48.93 | (45.03, | 46.70 | (43.02, | 2.22 | (−2.46, | 0.346 |
| | | (1.942) | 52.82) | (1.835) | 50.39) | (2.335) | 6.91) | |
| LD (18) | | 46.04 | (42.15, | | | −0.66 | (−5.35, | 0.778 |
| | | (1.942) | 49.94) | | | (2.335) | 4.03) | |
| M (19) | | 50.94 | (47.17, | | | 4.24 | (−0.35, | 0.069 |
| | | (1.882) | 54.72) | | | (2.284) | 8.83) | |

Stage N2 Duration (min)

| | | | | | | | | |
|---|---|---|---|---|---|---|---|---|
| HD (18) | P (20) | 147.83 | (132.04, | 144.07 | (129.12, | 3.76 | (−15.07, | 0.690 |
| | | (7.868) | 163.62) | (7.441) | 159.03) | (9.380) | 22.59) | |
| LD (18) | | 134.21 | (118.42, | | | −9.86 | (−28.69, | 0.298 |
| | | (7.868) | 150.00) | | | (9.380) | 8.97) | |
| M (19) | | 111.09 | (95.74, | | | −32.99 | (−51.40, | <.001** |
| | | (7.639) | 126.43) | | | (9.170) | −14.57) | |

Stage N3 %

| | | | | | | | | |
|---|---|---|---|---|---|---|---|---|
| HD (18) | P (20) | 29.36 | (25.89, | 25.83 | (22.55, | 3.53 | (−0.49, | 0.084 |
| | | (1.726) | 32.82) | (1.632) | 29.11) | (2.000) | 7.54) | |
| LD (18) | | 27.65 | (24.18, | | | 1.82 | (−2.19, | 0.367 |
| | | (1.726) | 31.12) | | | (2.000) | 5.84) | |
| M (19) | | 21.61 | (18.24, | | | −4.22 | (−8.15, | 0.036* |
| | | (1.674) | 24.98) | | | (1.956) | −0.29) | |

Note:
*,**,***indicate significance at 0.05, 0.001, and 0.0001 level, respectively.
Note:
PSG parameters were analyzed using a linear mixed effects model with fixed effects for sequence, period, and treatment, and a random effect for subject nested in sequence. Baseline PSG was included as a fixed-effect continuous variable in the model.
LD: Compound A 44 mg; HD: Compound A 112 mg; M: Modafinil 300 mg; P: Placebo.
Program Source\T_BIOPSG.sas (PRAHS SAS V9.4)

TABLE 26
Statistical Analysis of PSG Parameters
Pharmacodynamic Set

| | | Test | | Ref (Placebo) | | LS Mean Difference | | |
|---|---|---|---|---|---|---|---|---|
| | | LS Mean | | LS Mean | | Estimate | | |
| Test (N) | Ref (N) | (SE) | 95% CI | (SE) | 95% CI | (SE) | 95% CI | p-value |

Stage N3 Duration (min)

| | | | | | | | | |
|---|---|---|---|---|---|---|---|---|
| HD (18) | P (20) | 87.23 | (74.76, | 81.39 | (69.51, | 5.85 | (−7.77, | 0.393 |
| | | (6.189) | 99.71) | (5.876) | 93.26) | (6.780) | 19.46) | |
| LD (18) | | 81.05 | (68.57, | | | −0.34 | (−13.95, | 0.960 |
| | | (6.189) | 93.52) | | | (6.780) | 13.28) | |
| M (19) | | 51.61 | (39.47, | | | −29.78 | (−43.09, | <.001*** |
| | | (6.016) | 63.75) | | | (6.629) | −16.46) | |

Stage REM %

| | | | | | | | | |
|---|---|---|---|---|---|---|---|---|
| HD (18) | P (20) | 15.72 | (12.26, | 21.37 | (18.08, | −5.66 | (−9.36, | 0.003* |
| | | (1.714) | 19.18) | (1.631) | 24.67) | (1.841) | −1.96) | |
| LD (18) | | 20.70 | (17.24, | | | −0.68 | (−4.38, | 0.714 |
| | | (1.714) | 24.16) | | | (1.841) | 3.02 | |
| M (19) | | 17.46 | (14.08, | | | −3.92 | (−7.53, | 0.034* |
| | | (1.670) | 20.83) | | | (1.799) | −0.30) | |

Stage REM Duration (min)

| | | | | | | | | |
|---|---|---|---|---|---|---|---|---|
| HD (18) | P (20) | 46.83 | (35.72, | 66.56 | (56.02, | −19.73 | (−32.29, | 0.003* |
| | | (5.522) | 57.94) | (5.230) | 77.11) | (6.252) | −7.18) | |
| LD (18) | | 60.76 | (49.65, | | | −5.80 | (−18.35, | 0.358 |
| | | (5.522) | 71.87) | | | (6.252) | 6.75) | |
| M (19) | | 41.38 | (30.58, | | | −25.18 | (−37.46, | <.001** |
| | | (5.372) | 52.18) | | | (6.112) | −12.91) | |

Stage Wake %

| | | | | | | | | |
|---|---|---|---|---|---|---|---|---|
| HD (18) | P (20) | 11.14 | (6.30, | 10.30 | (5.74, | 0.84 | (−5.05, | 0.776 |
| | | (2.412) | 15.98) | (2.270) | 14.86) | (2.933) | 6.73 | |
| LD (18) | | 10.65 | (5.63, | | | 0.35 | (−5.70, | 0.907 |
| | | (2.509) | 15.68) | | | (3.013) | 6.41 | |
| M (19) | | 23.01 | (18.32, | | | 12.71 | (6.94, | <.001*** |
| | | (2.336) | 27.70) | | | (2.870) | 18.48) | |

Stage Wake Duration (min)

| | | | | | | | | |
|---|---|---|---|---|---|---|---|---|
| HD (18) | P (20) | 36.46 | (22.51, | 35.53 | (22.39, | 0.93 | (−16.12, | 0.913 |
| | | (6.961) | 50.41) | (6.552) | 48.68) | (8.493) | 17.97) | |
| LD (18) | | 35.55 | (21.60, | | | 0.02 | (−17.03, | 0.999 |
| | | (6.961) | 49.50) | | | (8.493) | 17.06) | |
| M (19) | | 60.92 | (47.41, | | | 25.39 | (8.70, | 0.004* |
| | | (6.737) | 74.43) | | | (8.313) | 42.08) | |

Note:
*,**,***indicate significance at 0.05, 0.001, and 0.0001 level, respectively.
Note:
PSG parameters were analyzed using a linear mixed effects model with fixed effects for sequence, period, and treatment, and a random effect for subject nested in sequence. Baseline PSG was included as a fixed-effect continuous variable in the model.
LD: Compound A 44 mg; HD: Compound A 112 mg; M: Modafinil 300 mg; P: Placebo.
Program Source\T_BIOPSG.sas (PRAHS SAS V9.4)

TABLE 27
Statistical Analysis of PSG Parameters
Pharmacodynamic Set

| | | Test | | Ref (Placebo) | | LS Mean Difference | | |
|---|---|---|---|---|---|---|---|---|
| | | LS Mean | | LS Mean | | Estimate | | |
| Test (N) | Ref (N) | (SE) | 95% CI | (SE) | 95% CI | (SE) | 95% CI | p-value |

Total Arousal

| | | | | | | | | |
|---|---|---|---|---|---|---|---|---|
| HD (18) | P (20) | 46.54 | (35.23, | 50.41 | (39.66, | −3.87 | (−16.41, | 0.538 |
| | | (5.618) | 57.85) | (5.327) | 61.15) | (6.248) | 8.67) | |
| LD (18) | | 47.56 | (36.25, | | | −2.85 | (−15.39, | 0.650 |
| | | (5.618) | 58.86) | | | (6.248) | 9.69) | |
| M (19) | | 51.11 | (40.09, | | | 0.70 | (−11.56, | 0.909 |
| | | (5.467) | 62.13) | | | (6.108) | 12.96) | |

Total Arousal Index

| | | | | | | | | |
|---|---|---|---|---|---|---|---|---|
| HD (18) | P (20) | 9.18 | (6.66, | 10.00 | (7.60, | −0.81 | (−3.72, | 0.577 |
| | | (1.257) | 11.71) | (1.189) | 12.39) | (1.449) | 2.10) | |
| LD (18) | | 10.07 | (7.55, | | | 0.08 | (−2.83, | 0.957 |
| | | (1.257) | 12.60) | | | (1.449) | 2.99) | |
| M (19) | | 14.28 | (11.82, | | | 4.28 | (1.43, | 0.004* |
| | | (1.222) | 16.73) | | | (1.417) | 7.12) | |

Total Leg Movements

| | | | | | | | | |
|---|---|---|---|---|---|---|---|---|
| HD (18) | P (20) | 16.94 | (8.31, | 18.94 | (10.79, | −2.01 | (−12.25, | 0.696 |
| | | (4.301) | 25.57) | (4.057) | 27.09) | (5.103) | 8.24) | |
| LD (18) | | 12.59 | (3.96, | | | −6.35 | (−16.59, | 0.219 |
| | | (4.301) | 21.22) | | | (5.103) | 3.89) | |
| M (19) | | 23.91 | (15.54, | | | 4.97 | (−5.05, | 0.324 |
| | | (4.167) | 32.28) | | | (4.989) | 14.99) | |

Total Leg Movements N1

| | | | | | | | | |
|---|---|---|---|---|---|---|---|---|
| HD (18) | P (20) | 1.41 | (0.33, | 1.26 | (0.24, | 0.15 | (−1.15, | 0.818 |
| | | (0.540) | 2.49) | (0.509) | 2.28) | (0.646) | 1.45) | |
| LD (18) | | 1.60 | (0.52, | | | 0.34 | (−0.96, | 0.605 |
| | | (0.540) | 2.68) | | | (0.646) | 1.63) | |
| M (19) | | 2.19 | (1.14, | | | 0.93 | (−0.34, | 0.148 |
| | | (0.523) | 3.24) | | | (0.632) | 2.20) | |

Total Leg Movements N2

| | | | | | | | | |
|---|---|---|---|---|---|---|---|---|
| HD (18) | P (20) | 7.59 | (2.30, | 8.54 | (3.56, | −0.95 | (−7.56, | 0.774 |
| | | (2.643) | 12.89) | (2.485) | 13.52) | (3.293) | 5.66) | |
| LD (18) | | 6.04 | (0.75, | | | −2.51 | (−9.11, | 0.450 |
| | | (2.643) | 11.33) | | | (3.293) | 4.10) | |
| M (19) | | 10.10 | (4.98, | | | 1.56 | (−4.91, | 0.630 |
| | | (2.555) | 15.22) | | | (3.223) | 8.03) | |

Note:
*,**,***indicate significance at 0.05, 0.001, and 0.0001 level, respectively.
Note:
PSG parameters were analyzed using a linear mixed effects model with fixed effects for sequence, period, and treatment, and a random effect for subject nested in sequence. Baseline PSG was included as a fixed-effect continuous variable in the model.
LD: Compound A 44 mg; HD: Compound A 112 mg; M: Modafinil 300 mg; P: Placebo.
Program Source\T_BIOPSG.sas (PRAHS SAS V9.4)

TABLE 28
Statistical Analysis of PSG Parameters
Pharmacodynamic Set

| | | Test | | Ref (Placebo) | | LS Mean Difference | | |
|---|---|---|---|---|---|---|---|---|
| | | LS Mean | | LS Mean | | Estimate | | |
| Test (N) | Ref (N) | (SE) | 95% CI | (SE) | 95% CI | (SE) | 95% CI | p-value |

Total Leg Movements N3

| | | | | | | | | |
|---|---|---|---|---|---|---|---|---|
| HD (18) | P (20) | 2.72 | (0.29, | 1.62 | (−0.68, | 1.09 | (−1.58, | 0.415 |
| | | (1.204) | 5.14) | (1.142) | 3.93) | (1.333) | 3.77) | |
| LD (18) | | 0.65 | (−1.77, | | | −0.97 | (−3.64, | 0.471 |
| | | (1.204) | 3.08) | | | (1.333) | 1.71) | |
| M (19) | | 2.61 | (0.25, | | | 0.99 | (−1.63, | 0.451 |
| | | (1.171) | 4.97) | | | (1.302) | 3.60) | |

Total Leg Movements REM

| | | | | | | | | |
|---|---|---|---|---|---|---|---|---|
| HD (18) | P (20) | 4.18 | (1.39, | 5.75 | (3.11, | −1.57 | (−4.76, | 0.329 |
| | | (1.390) | 6.98) | (1.314) | 8.40) | (1.592) | 1.63) | |
| LD (18) | | 3.44 | (0.65, | | | −2.31 | (−5.51, | 0.153 |
| | | (1.390) | 6.24) | | | (1.592) | 0.88) | |
| M (19) | | 5.75 | (3.04, | | | 0.00 | (−3.12, | 1.000 |
| | | (1.349) | 8.47) | | | (1.556) | 3.12) | |

Total Leg Movements with Awakening

| | | | | | | | | |
|---|---|---|---|---|---|---|---|---|
| HD (18) | P (20) | 0.84 | (−1.23, | 1.75 | (−0.20, | −0.91 | (−3.61, | 0.504 |
| | | (1.038) | 2.92) | (0.973) | 3.70) | (1.347) | 1.80) | |
| LD (18) | | 0.68 | (−1.40, | | | −1.07 | (−3.78, | 0.429 |
| | | (1.038) | 2.75) | | | (1.347) | 1.63) | |
| M (19) | | 3.14 | (1.14, | | | 1.39 | (−1.26, | 0.296 |
| | | (1.002) | 5.15) | | | (1.319) | 4.04) | |

Total Leg Movements with Microarous

| | | | | | | | | |
|---|---|---|---|---|---|---|---|---|
| HD (18) | P (20) | 2.14 | (0.84, | 2.01 | (0.79, | 0.13 | (−1.55, | 0.876 |
| | | (0.650) | 3.44) | (0.610) | 3.23) | (0.839) | 1.82) | |
| LD (18) | | 2.35 | (E05, | | | 0.34 | (−1.34, | 0.684 |
| | | (0.650) | 3.66) | | | (0.839) | 2.03) | |
| M (19) | | 3.44 | (2.19, | | | 1.43 | (−0.22, | 0.087 |
| | | (0.628) | 4.70) | | | (0.822) | 3.08) | |

Total Leg Movements with Resp Event

| | | | | | | | | |
|---|---|---|---|---|---|---|---|---|
| HD (18) | P (20) | 0.06 | (−0.13, | 0.05 | (−0.13, | 0.01 | (−0.24, | 0.952 |
| | | (0.095) | 0.25) | (0.089) | 0.23) | (0.123) | 0.25) | |
| LD (18) | | 0.16 | (−0.03, | | | 0.11 | (−0.14, | 0.388 |
| | | (0.095) | 0.35) | | | (0.123) | 0.35) | |
| M (19) | | 0.04 | (−0.14, | | | −0.01 | (−0.25, | 0.965 |
| | | (0.091) | 0.23) | | | (0.121) | 0.24) | |

Note:
*,**,***indicate significance at 0.05, 0.001, and 0.0001 level, respectively.
Note:
PSG parameters were analyzed using a linear mixed effects model with fixed effects for sequence, period, and treatment, and a random effect for subject nested in sequence. Baseline PSG was included as a fixed-effect continuous variable in the model.
LD: Compound A 44 mg; HD: Compound A 112 mg; M: Modafinil 300 mg; P: Placebo.
Program Source\T_BIOPSG.sas (PRAHS SAS V9.4)

TABLE 29
Statistical Analysis of PSG Parameters
Pharmacodynamic Set

| | | Test | | Ref (Placebo) | | LS Mean Difference | | |
|---|---|---|---|---|---|---|---|---|
| | | LS Mean | | LS Mean | | Estimate | | |
| Test (N) | Ref (N) | (SE) | 95% CI | (SE) | 95% CI | (SE) | 95% CI | p-value |

Total Leg Movements without Arousal

| | | | | | | | | |
|---|---|---|---|---|---|---|---|---|
| HD (18) | P (20) | 14.77 | (6.71, | 16.92 | (9.31, | −2.15 | (−11.79, | 0.655 |
| | | (4.017) | 22.82) | (3.787) | 24.53) | (4.799) | 7.48) | |
| LD (18) | | 10.21 | (2.15, | | | −6.71 | (−16.34, | 0.168 |
| | | (4.017) | 18.27) | | | (4.799) | 2.92) | |
| M (19) | | 20.42 | (12.61, | | | 3.50 | (−5.92, | 0.459 |
| | | (3.891) | 28.24) | | | (4.693) | 12.92) | |

Total Recording Time (min)

| | | | | | | | | |
|---|---|---|---|---|---|---|---|---|
| HD (18) | P (20) | 351.06 | (329.77, | 356.91 | (336.68, | −5.85 | (−29.35, | 0.619 |
| | | (10.56) | 372.34) | (10.01) | 377.13) | (11.70) | 17.65) | |
| LD (18) | | 342.50 | (321.22, | | | −14.40 | (−37.91, | 0.224 |
| | | (10.56) | 363.78) | | | (11.70) | 9.10) | |
| M (19) | | 321.98 | (301.26, | | | −34.92 | (−57.90, | 0.004* |
| | | (10.26) | 342.70) | | | (11.44) | −11.95) | |

Total Sleep Time (min)

| | | | | | | | | |
|---|---|---|---|---|---|---|---|---|
| HD (18) | P (20) | 298.98 | (271.50, | 309.47 | (283.38, | −10.49 | (−41.18, | 0.496 |
| | | (13.65) | 326.47) | (12.93) | 335.56) | (15.28) | 20.21) | |
| LD (18) | | 291.34 | (263.86, | | | −18.13 | (−48.82, | 0.241 |
| | | (13.65) | 318.83) | | | (15.28) | 12.57) | |
| M (19) | | 224.54 | (197.79, | | | −84.93 | (−115.0, | <.001*** |
| | | (13.27) | 251.29) | | | (14.94) | −54.91) | |

Wake After Persistent Sleep (min)

| | | | | | | | | |
|---|---|---|---|---|---|---|---|---|
| HD (18) | P (20) | 35.95 | (23.33, | 34.63 | (22.69, | 1.33 | (−13.52, | 0.858 |
| | | (6.290) | 48.58) | (5.936) | 46.56) | (7.395) | 16.17) | |
| LD (18) | | 33.78 | (21.15, | | | −0.85 | (−15.69, | 0.909 |
| | | (6.290) | 46.40) | | | (7.395) | 13.99) | |
| M (19) | | 47.63 | (35.38, | | | 13.00 | (−1.52, | 0.078 |
| | | (6.099) | 59.88) | | | (7.235) | 27.53) | |

Wake After Sleep Onset (min)

| | | | | | | | | |
|---|---|---|---|---|---|---|---|---|
| HD (18) | P (20) | 36.46 | (22.51, | 35.53 | (22.39, | 0.93 | (−16.12, | 0.913 |
| | | (6.961) | 50.41) | (6.552) | 48.68) | (8.493) | 17.97) | |
| LD (18) | | 35.55 | (21.60, | | | 0.02 | (−17.03, | 0.999 |
| | | (6.961) | 49.50) | | | (8.493) | 17.06) | |
| M (19) | | 60.92 | (47.41, | | | 25.39 | (8.70, | 0.004* |
| | | (6.737) | 74.43) | | | (8.313) | 42.08) | |

Note:
*,**,***indicate significance at 0.05, 0.001, and 0.0001 level, respectively.
Note:
PSG parameters were analyzed using a linear mixed effects model with fixed effects for sequence, period, and treatment, and a random effect for subject nested in sequence. Baseline PSG was included as a fixed-effect continuous variable in the model.
LD: Compound A 44 mg; HD: Compound A 112 mg; M: Modafinil 300 mg; P: Placebo.
Program Source\T_BIOPSG.sas (PRAHS SAS V9.4)

In the modafinil 300 mg treatment, there were multiple differences from the placebo group, consistent with effects on time to obtained persistent sleep (per. slp), less overall sleep, much lower sleep efficiency and more arousals, as noted in the following PSG parameters that were significantly different from placebo: time to per. slp (LS mean difference 19.32 min [95% CI 7.32, 31.32]), sleep efficiency (LS mean difference −18.44% [95% CI −25.16, −11.72]), spontaneous arousal index (LS mean difference 2.98 [95% CI 0.77, 5.19]), stage N1% (LS mean difference 4.05 [95% CI 1.58, 6.52]), stage N2 duration (LS mean difference −32.99 min [95% CI −51.40, −14.57]), stage N3% (LS mean difference −4.22 [95% CI −8.15, −0.29]), stage N3 duration (LS mean difference −29.78 [95% CI −43.09, −16.46]), stage REM % (LS mean difference −3.92 [95% CI −7.53, −0.30]), stage REM duration (LS mean difference −25.18 min [95% CI −37.46, −12.91]), stage wake % (LS mean difference 12.71 [95% CI 6.94, 18.48]), stage wake duration (LS mean difference 25.39 min [95% CI 8.70, 42.08]), total arousal index (LS mean difference 4.28 [95% CI 1.43, 7.12]), total recording time (LS mean difference −34.92 min [95% CI −57.90, −11.95]), total sleep time (LS mean difference −84.93 min [95% CI −115.0, −54.91]), and wake after sleep onset (LS mean difference 25.39 min [95% CI 8.70, 42.08]).

No statistically significant differences were observed in most sleep parameters for Compound A 44 mg, except for N2 latency (LS mean difference −2.74 min [95% CI −5.09, −0.38].

In the Compound A 112 mg treatment, the REM latency (LS mean difference 29.64 min [95% CI 5.44, 53.84]), stage REM % (LS mean difference −5.66 [95% CI −9.36, −1.96]), and stage REM duration (LS mean difference −19.73 min [95% CI −32.29, −7.18]), were statistically significantly different when compared to placebo. Stage REM % and stage REM duration changes were similar to that seen with modafinil.

However, sleep efficiency, arousals, and total sleep in both Compound A groups were not different from placebo, in contrast with the effects seen on recovery sleep with modafinil.

### Example I-7: PK/PD Conclusions

PD Conclusions
Mean MWT sleep latency post infusion start was 25.40, 38.82, and 30.87 minutes in the Compound A 44 mg, Compound A 112 mg, and modafinil treatments, respectively, compared to 8.61 minutes in the placebo treatment.
Mean increase in KSS from daytime to nighttime was lower for Compound A 44 and 112 mg and modafinil compared to placebo.
For subjects on 112 mg Compound A, the sleep latency on the MWT during infusion stayed at 40 minutes across several sessions, however, for subjects on 44 mg of Compound A, the sleep latency on the MWT decreased over the 4 sessions during infusion.

PK Conclusions
Compound A systemic exposure increased approximately dose proportionately between 44 and 112 mg dose levels.
After the IV infusion ended, Compound A plasma concentrations declined rapidly and in a biphasic manner with a mean terminal disposition phase t_{1/2z} of approximately 3 hours.

### CONCLUSIONS

Assay sensitivity was demonstrated, as modafinil 300 mg showed effects that were statistically significantly better than placebo on prolonging wakefulness in the MWT (placebo-adjusted average effect was 22.3 min [p<0.001]) and reducing sleepiness in the KSS (placebo-adjusted average effect was −2.5 [p<0.001]).

Both Compound A 44 mg and 112 mg showed effects that were statistically significantly better than placebo on prolonging wakefulness in the MWT (placebo-adjusted average effects were 16.8 and 30.2 min, respectively, with p-values<0.001) and reducing sleepiness in the KSS (placebo-adjusted average effects were −1.2 [p<0.001] and −3.3 [p<0.05], respectively).

Compound A systemic exposure increased approximately dose proportionately between 44 and 112 mg dose levels.

Compound A 44 and 112 mg regimens were well tolerated overall.

Incidence of AEs in the Compound A treatments was generally higher than in the placebo treatment, but most AEs were mild and no SAEs were observed.

A minimal effect on BP and pulse was observed with Compound A 44 mg. The effects on BP and pulse were similar between Compound A 112 mg and modafinil.

All the publications, patents, and the patent applications cited herein are incorporated herein by reference in their entireties.

### Example II-1: Human Multiple Dose Study to Evaluate Safety, Tolerability, Pharmacokinetics and Pharmacodynamics of Multiple Doses of an Orexin Type-2 Receptor (OX2R) Agonist in NT2 Patients

The purpose of the study was to evaluate the safety, tolerability, pharmacokinetics and pharmacodynamics of Compound A after multiple days of IV administration in patients with NT2. NT2 patients were evaluated in Cohort C1 and C2 (Table 30). Compound A or placebo were administered via IV infusion over 9 hours once daily for 7 days. A total of 14 patients with NT2 were treated with Compound A or placebo (5, 4, and 5 subjects in the placebo, Compound A 44 mg, and Compound A 112 mg groups, respectively). In this part, potential efficacy of Compound A was evaluated with MWT, ESS, KSS, night-time PSG (NPSG), and PGI-C as an exploratory PD assessment.

TABLE 30
Outline of the Study Parts and Dosing Cohorts

| Subject | Cohort/Panel | Daily dose level | Dosing regimen |
|---|---|---|---|
| NT2 | C1 | 44 mg | IV infusion over 9 hours |
| patients | C2 | 112 mg | for 7 days |

Abbreviations:
IV, intravenous;
NT2, narcolepsy type 2

Overview of Key Study Procedures

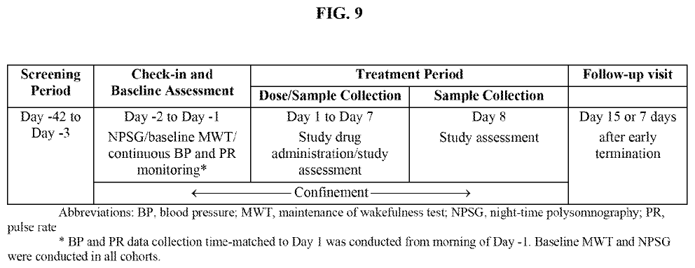

An overview of study schedule and PD testing in NT2 Patients is illustrated in FIG. 9. After screening, patients eligible for these parts had to discontinue any medication for narcolepsy including medications used for EDS or cataplexy. The medications had to be stopped for a minimum of 7 days or at least 5 half-lives of each medication, whichever was longer, before the first day of dosing (Day 1). In all cohorts of these parts, subjects underwent NPSG on Day −2 and baseline MWT sessions 4 times on Day −1 (at around 10:00, 12:00, 14:00, and 16:00). Study drug dosing via IV infusion was started at around 08:00 on Days 1-7. MWT was conducted on Day 1 at around 10:00, 12:00, 14:00, and 16:00. Subjects were allowed to take a nap on the days with no MWT assessment. At night of Day 6, the subjects underwent NPSG once again to evaluate the effect of multiple daytime dosing on night-time sleep structure. On Day 7, the subjects underwent MWT at the same hours as on Day −1. The subjects were discharged from the study site on Day 8.

Main Criteria for Selection of Study Population

Subject eligibility was confirmed according to the following criteria

Inclusion Criteria:
The patient must be aged 18 to 80 years, inclusive, at the time of informed consent.
The patient weighs at least 40 kg inclusive at Screening.
The patient must have a diagnosis of NT2, as defined by the International Classification of Sleep Disorders, Third Edition (ICSD-3).
The patient's Epworth sleepiness scale (ESS) is ≥10 at baseline.

Exclusion Criteria:
The patients consume excessive amounts, defined as greater than 6 servings (1 serving is approximately equivalent to 120 mg of caffeine) of coffee, tea, cola, energy drinks, or other caffeinated beverages per day.
The patients have a moderate to severe substance use disorder.
The patients have a risk of suicide according to endorsement of item 4 or 5 with Screening/Baseline visit C-SSRS or has made a suicide attempt in the previous 6 months.
The patients have a lifetime history of major psychiatric disorder, such as bipolar disorder or schizophrenia. Subject who has history of major depressive disorder (MDD) may be included but a subject having active MDD currently or in the past 6 months is excluded.
The patients experienced sleep wake cycle disturbance with external factors such as irregular work hours.

### Example II-2: Multiple-Dose PK of Intravenous Compound a in Patients with NT2

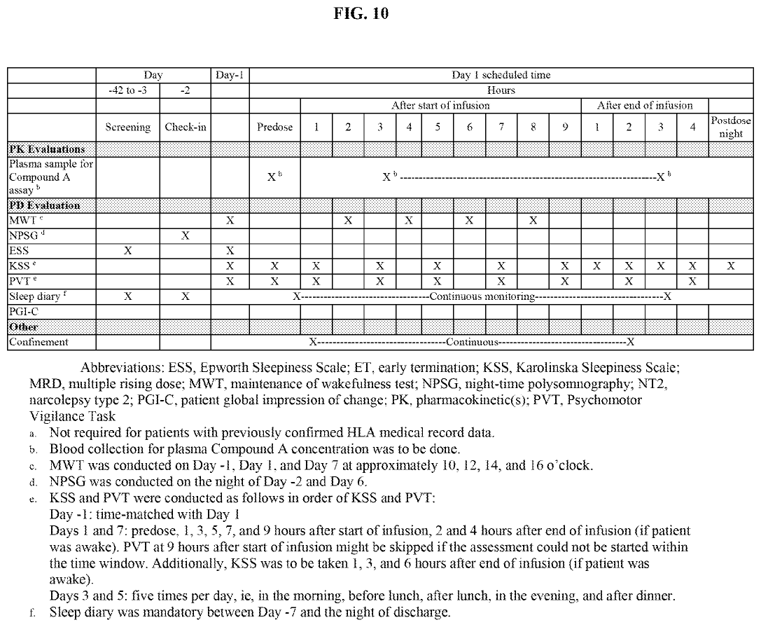

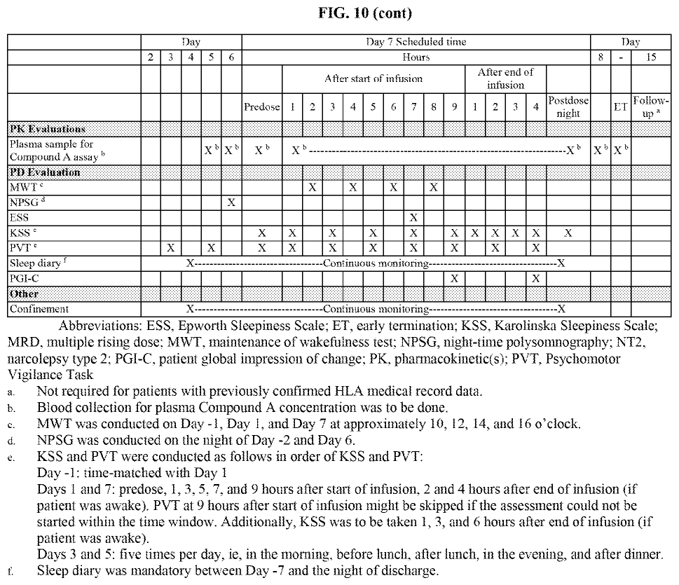

Blood samples used for PK analysis of Compound A was collected according to Table 31. See also FIG. 10 for whole schedules of study procedures.

TABLE 31
Blood Sampling for Pharmacokinetic analysis (NT2 Patients)

| Analyzed | | Study | |
|---|---|---|---|
| substances | Samples | date | Blood sampling time |
| Compound A | Plasma | Days | C1 and C2 |
| | | 1-8 | Days 1 and 7: Predose, 1, 2, 4, 6, |
| | | | and 9 hours after start of infusion, |
| | | | 0.17, 0.5, 2, 6, 10, and 15 hours |
| | | | after end of infusion. |
| | | | C1 and C2 |
| | | | Days 5 and 6: Predose, 9 hours |
| | | | after start of infusion |

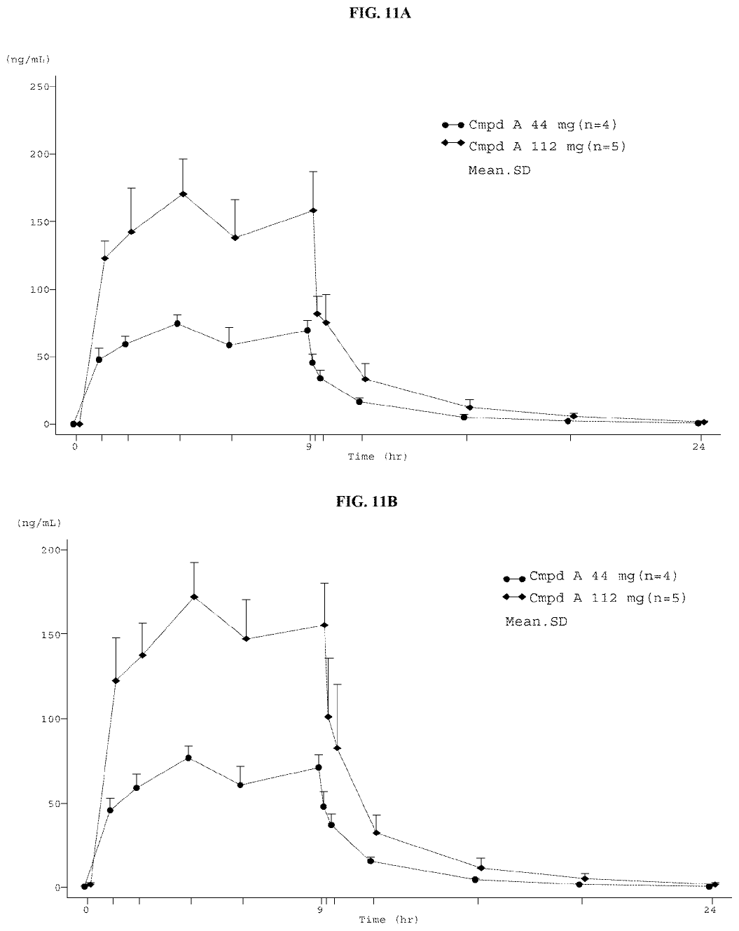

Summaries of plasma concentrations of Compound A are presented in Table 32. Mean and standard deviation plots of plasma Compound A concentrations on Day 1 and Day 7 are presented in FIGS. 11A and B, respectively. Summaries of PK parameters of Compound A on Day 1 and Day 7 are presented in Table 33 and Table 34 respectively. Based on R_{ac(AUC)} and R_{ac(Cmax)} values of approximately 1, no drug accumulation with QD dosing after a 9-hour IV infusion daily for 7 days was observed.

TABLE 32
Summary of Plasma Concentration by Visit by Treatment Group (Cohort C1, C2) (PK set)

| | | Visit | | | | | |
|---|---|---|---|---|---|---|---|
| | | | 1 Hour | 2 Hours | 4 Hours | 6 Hours | 9 Hours |
| Analyte/ | | Predose | Postdose | Postdose | Postdose | Postdose | Postdose |
| Treatment | Statistics | at Day 1 | at Day 1 | at Day 1 | at Day 1 | at Day 1 | at Day 1 |
| Cmpd A (ng/mL) | N | 4 | 4 | 4 | 4 | 4 | 4 |
| Cmpd A 44 mg | Mean | 0.000 | 47.40 | 59.05 | 74.13 | 50.05 | 69.05 |
| | SD | 0.0000 | 9.1917 | 6.2386 | 6.6445 | 13.490 | 7.3623 |
| Cmpd A 112 mg | N | 5 | 5 | 5 | 5 | 5 | 5 |
| | Mean | 0.000 | 122.6 | 142.5 | 170.4 | 138.2 | 150.2 |
| | SD | 0.0000 | 13.293 | 32.114 | 25.755 | 27.878 | 25.210 |

| | | Visit | | | | |
|---|---|---|---|---|---|---|
| | | 0.17 Hour | 0.5 Hour | 2 Hours | 6 Hours | 10 Hours |
| | | after End | after End | after End | after End | after End |
| Analyte/ | | of Infusion | of Infusion | of Infusion | of Infusion | of Infusion |
| Treatment | Statistics | at Day 1 | at Day 1 | at Day 1 | at Day 1 | at Day 1 |
| Cmpd A (ng/mL) | N | 4 | 4 | 4 | 4 | 4 |
| Cmpd A 44 mg | Mean | 45.48 | 33.98 | 16.33 | 5.020 | 2.515 |
| | SD | 6.4717 | 6.1419 | 2.9307 | 1.8060 | 0.47592 |
| Cmpd A 112 mg | N | 5 | 5 | 5 | 5 | 5 |
| | Mean | 81.03 | 75.26 | 32.46 | 12.03 | 5.610 |
| | SD | 12.578 | 20.870 | 11.337 | 5.9786 | 2.3393 |

| | | Visit | | | | | |
|---|---|---|---|---|---|---|---|
| | | 14 Hours | | | | | |
| | | after End | | 9 Hours | | 9 Hours | |
| Analyte / | | of Infusion | Predose | Postdose | Predose | Postdose | Predose |
| Treatment | Statistics | at Day 1 | at Day 5 | at Day 5 | at Day 6 | at Day 6 | at Day 7 |
| Cmpd A (ng/mL) | N | 4 | 4 | 4 | 4 | 4 | 4 |
| Cmpd A 44 mg | Mean | 0.7016 | 0.9273 | 73.15 | 0.7163 | 65.80 | 0.5635 |
| | SD | 0.18688 | 0.40396 | 0.3686 | 0.47250 | 6.3156 | 0.29104 |
| Cmpd A 113 mg | N | 5 | 5 | 5 | 5 | 5 | 5 |
| | Mean | 1.514 | 1.820 | 162.4 | 1.684 | 162.2 | 1.601 |
| | SD | 1.0115 | 1.2506 | 31.911 | 1.3258 | 34.325 | 1.2829 |

| | | Visit | | | | |
|---|---|---|---|---|---|---|
| | | 1 Hours | 2 Hours | 4 Hours | 6 Hours | 9 Hours |
| Analyte / | | Postdose | Postdose | Postdose | Postdose | Postdose |
| Treatment | Statistics | at Day 7 | at Day 7 | at Day 7 | at Day 7 | at Day 7 |
| Cmpd A (ng/mL) | N | 4 | 4 | 4 | 4 | 4 |
| Cmpd A 44 mg | Mean | 45.63 | 59.00 | 76.70 | 60.95 | 71.15 |
| | SD | 7.1723 | 8.2369 | 7.2310 | 10.602 | 7.4375 |
| Cmpd A 113 mg | N | 5 | 5 | 5 | 5 | 5 |
| | Mean | 122.8 | 137.8 | 172.4 | 147.6 | 155.4 |
| | SD | 25.412 | 18.992 | 20.305 | 23.104 | 24.876 |

| | | Visit | | | | | |
|---|---|---|---|---|---|---|---|
| | | 0.17 Hour | 0.5 Hour | 2 Hours | 6 Hours | 10 Hours | 15 Hours |
| | | after End | after End | after End | after End | after End | after End |
| Analyte / | | of Infusion | of Infusion | of Infusion | of Infusion | of Infusion | of Infusion |
| Treatment | Statistics | at Day 7 | at Day 7 | at Day 7 | at Day 7 | at Day 7 | at Day 7 |
| Cmpd A (ng/mL) | N | 4 | 4 | 4 | 4 | 4 | 4 |
| Cmpd A 44 mg | Mean | 47.73 | 37.00 | 15.65 | 4.440 | 1.763 | 0.5318 |
| | SD | 8.8703 | 6.3943 | 2.3345 | 0.69153 | 0.74038 | 0.27555 |
| Cmpd A 112 mg | N | 5 | 5 | 5 | 5 | 5 | 5 |
| | Mean | 101.0 | 87.90 | 32.16 | 11.67 | 5.440 | 1.563 |
| | SD | 35.025 | 37.476 | 10.696 | 5.5454 | 2.6071 | 1.0423 |

Note:
The 0.17 hours postdose data on Day 1 of one subject dosed 112 mg was excluded because of suspect of contamination.

TABLE 33
Summary of Pharmacokinetic Parameters of Compound
A (Cohort C1, C2) on Day 1 (PK set)

| | | | | N of | | | |
|---|---|---|---|---|---|---|---|
| Variable | Treatment | N | Mean | Geo Mean | Geo mean | SD | CV |
| AUClast | Cmpd A 44 mg | 4 | 652.0 | 4 | 648.1 | 80.519 | 12.3 |
| (h*ng/mL) | Cmpd A 112 mg | 5 | 1516 | 5 | 1496 | 279.16 | 18.4 |
| AUCinf | Cmpd A 44 mg | 4 | 655.0 | 4 | 651.2 | 79.796 | 12.2 |
| (h*ng/mL) | Cmpd A 112 mg | 5 | 1520 | 5 | 1500 | 283.99 | 18.7 |
| AUCtau | Cmpd A 44 mg | 4 | 652.3 | 4 | 648.3 | 80.748 | 12.4 |
| (h*ng/mL) | Cmpd A 112 mg | 5 | 1516 | 5 | 1496 | 279.16 | 18.4 |
| Cmax | Cmpd A 44 mg | 4 | 74.13 | 4 | 73.89 | 6.6445 | 9.0 |
| (ng/mL) | Cmpd A 112 mg | 5 | 172.2 | 5 | 171.0 | 22.610 | 13.1 |
| Ceoi | Cmpd A 44 mg | 4 | 69.05 | 4 | 68.74 | 7.3623 | 10.7 |
| (ng/mL) | Cmpd A 112 mg | 5 | 158.2 | 5 | 156.1 | 29.210 | 18.5 |

| Variable | Treatment | N | Mean | SD |
|---|---|---|---|---|
| tmax (h) | Cmpd A 44 mg | 4 | 3.875 | 0.1443 |
| | Cmpd A 112 mg | 5 | 4.926 | 2.2130 |

| Variable | Treatment | N | Mean | SD | CV |
|---|---|---|---|---|---|
| t½z (h) | Cmpd A 44 mg | 4 | 2.708 | 0.2133 | 7.9 |
| | Cmpd A 112 mg | 5 | 2.800 | 0.6642 | 23.7 |
| Vss (L) | Cmpd A 44 mg | 4 | 122.2 | 25.564 | 20.9 |
| | Cmpd A 112 mg | 5 | 121.3 | 32.950 | 27.2 |
| Vz (L) | Cmpd A 44 mg | 4 | 287.5 | 56.783 | 21.2 |
| | Cmpd A 112 mg | 5 | 298.8 | 46.997 | 15.7 |
| CL (L/h) | Cmpd A 44 mg | 4 | 68.00 | 8.9870 | 13.2 |
| | Cmpd A 112 mg | 5 | 75.66 | 13.241 | 17.5 |
| Cmax D | Cmpd A 44 mg | 4 | 1.685 | 0.14799 | 8.8 |
| (ng/mL/mg) | Cmpd A 112 mg | 5 | 1.540 | 0.20224 | 13.1 |
| AUCtau D | Cmpd A 44 mg | 4 | 14.83 | 1.8464 | 12.5 |
| (h*ng/mL/mg) | Cmpd A 112 mg | 5 | 13.52 | 2.5054 | 18.5 |

TABLE 34
Summary of Pharmacokinetic Parameters of Compound
A (Cohort C1, C2) on Day 7 (PK set)

| | | | | N of | | | |
|---|---|---|---|---|---|---|---|
| Variable | Treatment | N | Mean | Geo Mean | Geo Mean | SD | CV |
| AUClast | Cmpd A 44 mg | 4 | 660.0 | 4 | 657.1 | 72.833 | 11.0 |
| (h*ng/mL) | Cmpd A 112 mg | 5 | 1534 | 5 | 1519 | 248.46 | 16.2 |
| UCtau | Cmpd A 44 mg | 4 | 660.0 | 4 | 657.1 | 72.833 | 11.0 |
| (h*ng/mL) | Cmpd A 112 mg | 5 | 1534 | 5 | 1519 | 248.46 | 16.2 |
| Cmax (mg/mL) | Cmpd A 44 mg | 4 | 76.70 | 4 | 76.44 | 7.2310 | 9.4 |
| | Cmpd A 112 mg | 5 | 173.4 | 5 | 172.3 | 31.244 | 12.3 |
| Cmin (mg/mL) | Cmpd A 44 mg | 4 | 0.5233 | 4 | 0.4542 | 0.30402 | 53.1 |
| | Cmpd A 112 mg | 5 | 1.523 | 5 | 1.192 | 1.0535 | 69.2 |
| Ceoi (mg/mL) | Cmpd A 44 mg | 4 | 71.15 | 4 | 70.87 | 7.4375 | 10.5 |
| | Cmpd A 112 mg | 5 | 155.4 | 5 | 153.9 | 24.876 | 16.0 |

| Variable | Treatment | N | Mean | SD | CV |
|---|---|---|---|---|---|
| PTR | Cmpd A 44 mg | 4 | 202.0 | 130.03 | 64.4 |
| | Cmpd A 112 mg | 5 | 201.1 | 210.74 | 104.8 |
| Rac AUC | Cmpd A 44 mg | 4 | 1.017 | 0.098127 | 9.7 |
| | Cmpd A 112 mg | 5 | 1.018 | 0.030760 | 3.0 |
| Rac Cmax | Cmpd A 44 reg | 4 | 1.035 | 0.050169 | 4.8 |
| | Cmpd A 112 mg | 5 | 1.009 | 0.032784 | 3.3 |

| Variable | Treatment | N | Mean | SD |
|---|---|---|---|---|
| tmax (h) | Cmpd A 44 mg | 4 | 3.908 | 0.1312 |
| | Cmpb A 112 mg | 5 | 4.924 | 2.2085 |

| Variable | Treatraent | N | Mean | SD | CV |
|---|---|---|---|---|---|
| t½z (h) | Cmpd A 44 mg | 4 | 2.643 | 0.3625 | 13.7 |
| | Cmpd A 112 mg | 5 | 3.136 | 0.7548 | 24.1 |
| Vss (L) | Cmpd A 44 mg | 4 | 114.5 | 30.506 | 26.6 |
| | Cmpd A 112 mg | 5 | 123.5 | 23.163 | 20.4 |
| Vz (L) | Cmpd A 44 mg | 4 | 258.3 | 56.346 | 21.8 |
| | Cmpd A 112 mg | 5 | 338.0 | 93.683 | 27.7 |
| CL (L/h) | Cmpd A 44 mg | 4 | 67.25 | 6.8927 | 10.2 |
| | Cmpd A 112 mg | 5 | 74.46 | 11.332 | 15.2 |
| Cmax D | Cmpd A 44 mg | 4 | 1.745 | 0.16422 | 9.4 |
| (ng/mL/mg) | Cmpd A 112 mg | 5 | 1.550 | 0.18960 | 12.2 |
| AUCtau D | Cmpd A 44 mg | 4 | 15.00 | 1.6269 | 10.8 |
| (h*ng/mL/mg) | Cmpd A 112 mg | 5 | 13.68 | 2.2163 | 16.2 |

### Example II-3: MWT

The MWT is a validated objective measure that evaluates a person's ability to remain awake under soporific conditions for a defined period of time. As there is no biological measure of wakefulness, this was measured indirectly by the inability or delayed tendency to fall asleep. This tendency to fall asleep was measured via EEG-derived sleep latency in MWT.

On Day −1, Day 1 and Day 7, 40-minute session (1 session) of MWTs was performed 4 times (approximately at 10:00, 12:00, 14:00, and 16:00) per day. Sleep latency of each session was recorded. Subjects were required to stay awake during an interval between sessions.

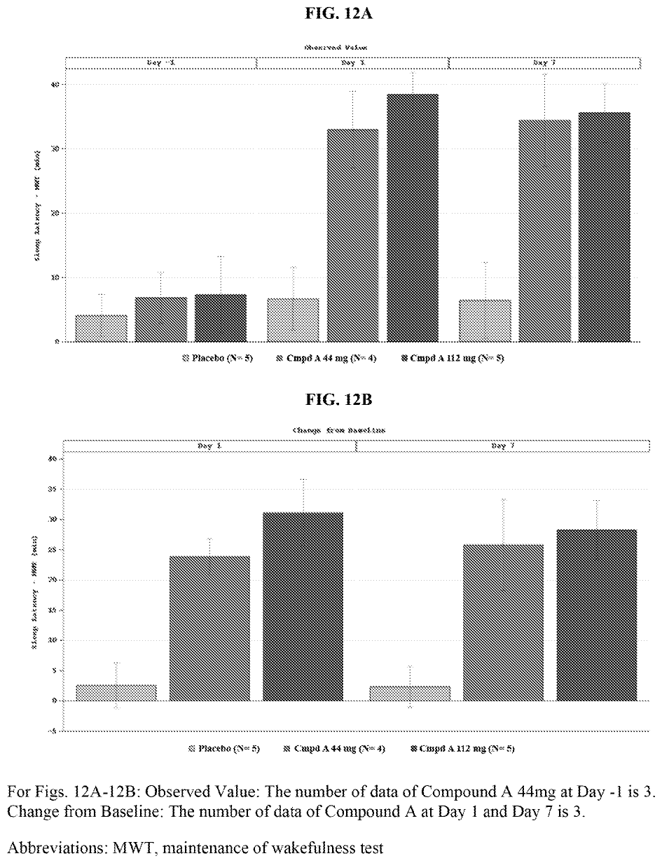

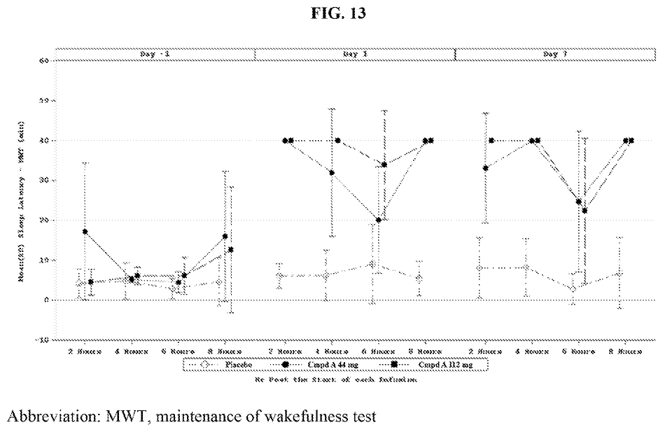

Average sleep latency in MWT and change from baseline by visit are displayed in FIGS. 12A and 12B, respectively. Mean and standard deviation plots of sleep latency in each session in MWT by visit are shown in FIG. 13.

In patients with NT2, the average sleep latency in MWT increased in the Compound A 44 mg group and Compound A 112 mg group compared with the placebo group. On Day 1, the mean sleep latency was 33.03 minutes and 38.48 minutes in the Compound A 44 mg group and Compound A 112 mg group, respectively, compared with 6.70 minutes in the placebo group. On Day 7, the mean sleep latency was 34.47 minutes and 35.60 minutes in the Compound A 44 mg group and Compound A 112 mg group, respectively, compared with 6.48 minutes in the placebo group. The mean changes from baseline in average sleep latency in MWT (min) were 2.58, 23.88, and 31.15 on Day 1 and 2.35, 25.79, and 28.28 on Day 7 in the placebo, Compound A 44 mg, and Compound A 112 mg groups, respectively.

During 9-hour IV infusion of Compound A, a significant increase in sleep latency was observed in all active groups compared with placebo. The maximum sleep latency of 40 minutes was achieved in the Compound A 44 mg group at 2 and 8 hours on Day 1 and at 4 and 8 hours on Day 7, and in the Compound A 112 mg group at 2, 4, and 8 hours both on Day 1 and Day 7.

### Example II-4: ESS

Summaries of ESS and change from baseline are presented in

Table 35.

In patients with NT2, ESS was improved in the Compound A 44 mg and Compound A 112 mg groups compared with the placebo group. On Day 7, the mean ESS score was 13.3 and 6.0 in the Compound A 44 mg group and Compound A 112 mg group, respectively, compared with 16.2 in the placebo group. The mean changes from baseline in ESS were −1.4, −3.8, and −12.2 on Day 7 in the placebo, Compound A 44 mg, and Compound A 112 mg groups, respectively.

TABLE 35
Summary of ESS and Change from Baseline by Visit (Cohorts C1, C2) (PD Set)

| Placebo | | Compound A 44 mg | | Compound A 112 mg | |
|---|---|---|---|---|---|
| (N = 5) | | (N = 4) | | (N = 5) | |
| | Change | | Change | | Change |
| Observed | from | Observed | from | Observed | from |
| Value | Baseline | Value | Baseline | Value | Baseline |

Day −1

| | | | |
|---|---|---|---|
| N | 5 | 4 | 5 |
| Mean | 17.6 | 17.0 | 18.2 |
| SD | 3.05 | 2.16 | 2.17 |
| Median | 17.0 | 17.5 | 18.0 |
| Min, max | 14, 22 | 14, 19 | 15, 21 |

Day 7

| | | | | | | |
|---|---|---|---|---|---|---|
| N | 5 | 5 | 4 | 4 | 5 | 5 |
| Mean | 16.2 | −1.4 | 13.3 | −3.8 | 6.0 | −12.2 |
| SD | 3.96 | 2.51 | 5.62 | 3.50 | 5.83 | 6.30 |
| Median | 16.0 | 0.0 | 14.0 | −3.5 | 4.0 | −13.0 |
| Min, max | 11, 22 | −5, 1 | 6, 19 | −8, 0 | 0, 14 | −21, −4 |

Abbreviation:
ESS, Epworth Sleepiness Scale

### Example II-5: KSS

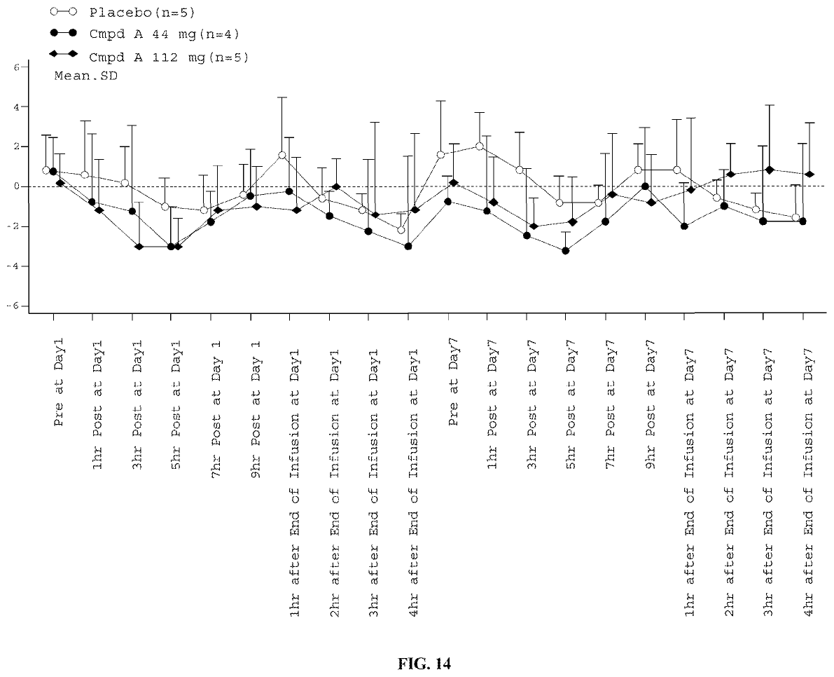

Mean and standard deviation plots of change from time-matched baseline in KSS are provided in FIG. 14.

In patients with NT2, lower KSS values in active groups was observed during infusion. The mean changes from baseline in KSS at 9 hours postdose were −0.4, −0.5, and −1.0 on Day 1 and 0.8, 0.0, and −0.8 on Day 7 in the placebo, Compound A 44 mg, and Compound A 112 mg groups, respectively.

### CONCLUSIONS

Compound A was safe and well tolerated in the multiple IV administrations of up to 112 mg for patients with NT2. There was no SAE, and no TEAE leading to study drug discontinuation. Dose dependent increase in frequency of TEAEs and drug-related TEAEs was generally observed.

A trend for the observed BP and PR increases with increasing Compound A plasma exposure was observed in NT2 patient populations. There was 1 TEAE related to BP (blood pressure increased in Compound A 112 mg group), which was of mild intensity and did not lead to drug discontinuation.

Following daily 9-hour IV infusions, mean plasma systemic exposures of Compound A, which were similar between healthy adults and the patients with NT2, generally increased in a dose proportional manner over the dose range studied. Consistent with the short t_{1/2z}, no drug accumulation was observed with QD dosing of 9-hour IV infusions.

In patients with NT2, the mean sleep latency of MWT (min) on Day 7 was 34.47 and 35.60 in the Compound A 44 mg group and the Compound A 112 mg group, respectively, compared with 6.48 in the placebo group. The maximum sleep latency, ie, 40 minutes of wakefulness, was achieved in the Compound A 44 mg group at 2 and 8 hours after start of IV infusion on Day 1 and at 4 and 8 hours on Day 7, and in the Compound A 112 mg group at 2, 4, and 8 hours both on Day 1 and Day 7.

The results of ESS/KSS, PGI-C and number of naps during the day generally supported the wakefulness PD effect observed in MWT.

### Example III-1: Human Study to Evaluate Safety, Tolerability, Pharmacokinetics and Pharmacodynamics of a Single Dose of an Orexin Type-2 Receptor (OX2R) Agonist in Subjects with Obstructive Sleep Apnea Who were Experiencing Excessive Daytime Sleepiness Despite Adequate Use of Continuous Positive Airway Pressure (CPAP)

The purpose of the study was to evaluate the pharmacokinetics (PK), pharmacodynamics (PD), safety and tolerability of a single intravenous (IV) dose of Compound A in adults who have been diagnosed with obstructive sleep apnea (OSA) and who are experiencing excessive daytime sleepiness (EDS) despite adequate use of CPAP. Twenty-five patients were randomized, and 23 patients completed the study. All patients at entry were having EDS despite being adequately controlled by CPAP for their sleep apnea.

The treatment periods began on Day 1 of Treatment Period 1 (Study Day 1) with Treatment Periods 2 and 3 commencing on Study Days 3 and 5, respectively. In the morning of Day 1 of Treatment Period 1, eligible subjects were randomized to 1 of the 6 sequence groups as listed in Table 36. After randomization, the subject was dosed in 3 treatment periods according to the order defined by the sequence group to which he/she was randomized. On Day 1 of each treatment period, Compound A or placebo was administered as a single 9-hour IV infusion commencing at approximately 08:00. The infusion was terminated at approximately 17:00. On Day 1 of each treatment period, four 40-minute maintenance of wakefulness test (MWT) sessions was performed at 2, 4, 6, and 8 hours after the start of the infusion. Subjects were required to stay awake in between the MWT sessions.

TABLE 36
Schematic of Study Design

| Sequence | Treatment Period 1 | Treatment Period 2 | Treatment Period 3 |
|---|---|---|---|
| 1 | Compound A 44 mg | Placebo | Compound A 112 mg |
| 2 | Compound A 112 mg | Compound A 44 mg | Placebo |
| 3 | Placebo | Compound A 112 mg | Compound A 44 mg |
| 4 | Compound A 112 mg | Placebo | Compound A 44 mg |
| 5 | Placebo | Compound A 44 mg | Compound A 112 mg |
| 6 | Compound A 44 mg | Compound A 112 mg | Placebo |

On Day 1 of Treatment Period 1, all eligible subjects were randomized and received the first dose of study drug. Compound A or placebo was administered as a single 9-hour IV infusion commencing at approximately 08:00. On each dosing day (Study Days 1, 3, and 5), 40-minute MWT sessions were performed at 2, 4, 6, and 8 hours after the start of the infusion. There was a minimum 24-hour washout interval between the end of the infusion and the commencement of treatment in subsequent treatment periods to allow for complete elimination of the preceding treatment effect. The KSS and PVT were collected per the Schedule of Study Procedures. Blood samples for determination of Compound A plasma concentrations were collected predose and at specified time points postdose, Safety assessments were recorded. During the study, AEs were recorded, clinical laboratory tests, vital signs, and safety ECGs were obtained. Study Days 2, 4, and 6 were washout days. Subjects were discharged from the unit following completion of study exit procedures in the afternoon of Study Day 6 (Day 2 of Treatment Period 3). Subjects were contacted by telephone approximately 7 days (±2 days) following unit discharge for a safety check. An overview of inpatient unit study schedule is shown in Table 37.

TABLE 37
Overview of inpatient unit study schedule

| Screening | Check-in and | Treatment Periods 1 to 3 | | End of |
|---|---|---|---|---|
| Period | Baseline | Dosing | Washout | Study Visit |
| Study Days −28 | Study Days −2 | Study Days 1, 3, | Study Days 2, 4, | Study Day 13 (±2 days) |
| to −3 | (NPSG ^{a}) | and 5 Study drug | and 6 ^{b} PK and | Follow-up (by phone |
| | and −1 (baseline) | administration/ | safety assessments | or if needed in clinic) |
| | | PD, PK, and | | |
| | | safety assessments | | |

← Confinement Study Day −2 to Day 6 →

NPSG: nocturnal polysomnography;
PD: pharmacodynamic;
PK: pharmacokinetic
^{a} NPSG only on Study Day −2.
^{b} Discharge from unit on Study Day 6.

Subject Population

Male and Female Subjects Diagnosed as OSA but are Experiencing EDS with Current Use of CPAP as Primary OSA Therapy

Main Criteria for Inclusion
Has OSA diagnosed according to the international classification of sleep disorders-3 (ICSD-3) criteria and with current use of CPAP.
Has a complaint of EDS despite “consistent use” of CPAP as defined by machine tracking time as having at least 4 hours of CPAP use/night on at least 70% during the approximately 1 month before randomization.
If taking a stimulant medication for the treatment of excessive daytime sleepiness must be willing to discontinue medication before randomization into the study.
Has a regular bedtime between 21:00 and 24:00 as verified by history and regular time in bed averaging between 7.5 and 9.0 hours/night and gets at least 6.5 hours/night on average of sleep, as defined by approximately 7 days of actigraphy supported by a sleep diary, which are completed at least 1 week before Study Day-2.
Has a Epworth sleepiness scale (ESS) score of ≥10 at screening and Study Day −2, with or without stimulants.
Nocturnal polysomnography (NPSG) demonstrates that the participant does not have other comorbid sleep disorders or clinically significant nocturnal hypoxemia (O2 saturation≤80% for ≥5% of total sleep time) and that their apnea-hypopnea index (AHI) is ≤10.
Has an average (of 4 sessions) baseline MWT sleep latency less than or equal to 20 minutes and no single session has a sleep latency of greater than 30 minutes as determined by the site investigator.

Main Criteria for Exclusion
Has supine or standing average systolic blood pressure (SBP)≥140 millimeters of mercury (mm Hg) or average diastolic blood pressure (DBP)≥90 mm Hg at screening or Study Day-2; blood pressures will be averaged over 3 readings done 10 minutes (min) apart.
A screening electrocardiogram (ECG) reveals a QT interval with Fridericia correction method>450 milliseconds (ms) (men) or >470 ms (women).
Has a usual bedtime later than 01:00 or an occupation requiring nighttime shift work or variable shift work within the past 6 months or travel with significant jet lag within 14 days before Study Day-2.
Short sleepers with chronic sleep deprivation who get on average less than 7.5 hours/night time in bed and/or less than 6.5 hours of sleep per night as defined by approximately 1 week of nocturnal actigraphy testing and supported by a sleep diary, both of which are completed at least 1 week before Study Day −2 admission to the clinical unit.
Has a history of a sleep disorder other than OSA that is associated with EDS on the basis of interviews at the screening visit, such as, for example, restless legs syndrome, confirmed by prior pretreatment polysomnography (PSG) data demonstrating periodic limb movement during sleep (PLMS)>15.
Has used any over-the-counter (OTC) or prescription medications with stimulating properties within 7 days before dosing or 5 half-lives (whichever is longer) that could affect the evaluation of EDS or any use of sodium oxybate within 3 months of screening.
Has nicotine dependence that is likely to have an effect on sleep (e.g., a participant who routinely awakens at night to smoke) or challenge the conduct of this study (smokes≥10 cigarettes/day) and/or the participant is unwilling to discontinue all smoking and nicotine use during the study.
Has a caffeine consumption of more than 600 mg/day for 7 days before Study Day-1 (1 serving of coffee is approximately equivalent to 120 mg of caffeine).
History or presence of any acutely unstable medical condition, behavioral or psychiatric disorder (including active suicidal ideation), or surgical history that could affect the safety of the subject or interfere with study efficacy, safety, PK assessments, or the ability of the subject to complete the study per the judgment of the investigator.

### Example III-2: Compound a Single Dose PK in Subjects with OSA

Blood samples used for PK analysis of Compound A was collected according to Table 38.

TABLE 38
Blood Sampling for Pharmacokinetic analysis

| Analyzed | | | |
|---|---|---|---|
| Substances | Samples | Study Date | Blood Sampling Time |
| Cmpd A | Plasma | Days 1-2 of | Day 1: predose, 1, 3, 5, 7, and 9 |
| | | each treatment | hours after start of infusion, and |
| | | period | at 0.17, 1, 4, and 15 hours after |
| | | | each end point. |

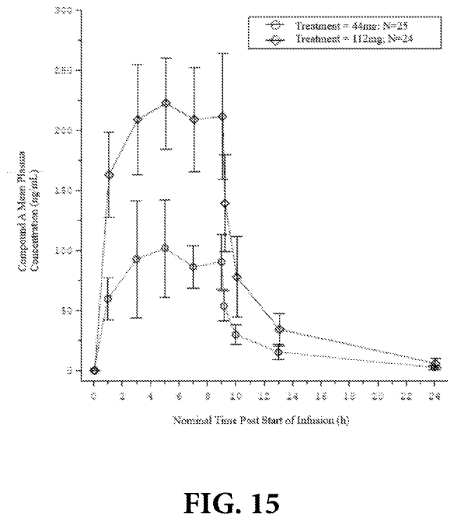

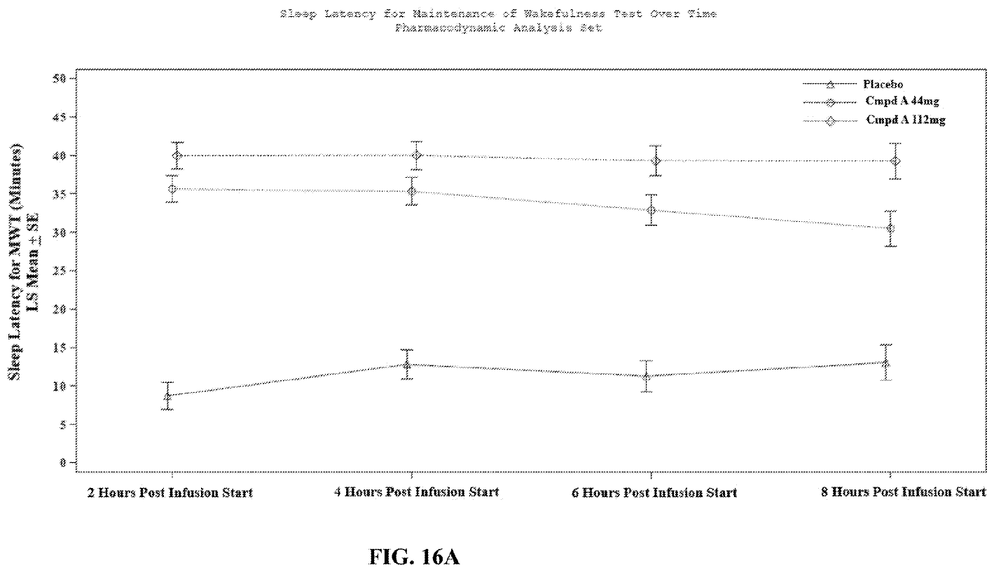

Summary of plasma concentrations of Compound is presented in Table 39. Mean and standard deviation plots of plasma Compound A concentrations are presented in FIG. 15. Summary of PK parameters of Compound A is presented in Table 40.

TABLE 39
Summary of Plasma Concentration by Visit by Treatment Group (PK set)

| | Nominal Time (h) | | | | | | | | | |
|---|---|---|---|---|---|---|---|---|---|---|
| Treatment/ | After Start of Infusion | | | | | | From End of Infusion | | | |
| Statistics | 0 | 1 | 3 | 5 | 7 | 9 | 0.17 | 1 | 4 | 15[a] |

Cmpd A (ng/mL) 44 mg (N = 25)

| | | | | | | | | | | |
|---|---|---|---|---|---|---|---|---|---|---|
| n | 25 | 24 | 25 | 25 | 25 | 22 | 18 | 24 | 24 | 25 |
| Mean | BLQ | 59.80 | 92.43 | 101.7 | 86.37 | 90.42 | 53.69 | 29.90 | 15.22 | 2.462 |
| SD | ND | 17.72 | 48.45 | 40.63 | 17.42 | 22.86 | 12.38 | 8.039 | 6.229 | 1.502 |

Cmpd A (ng/mL) 112 mg (N = 24)

| | | | | | | | | | | |
|---|---|---|---|---|---|---|---|---|---|---|
| n | 23 | 24 | 24 | 23 | 24 | 18 | 15 | 21 | 22 | 24 |
| Mean | BLQ | 162.9 | 208.7 | 222.3 | 208.9 | 211.6 | 139.3 | 77.87 | 34.12 | 5.962 |
| SD | ND | 35.32 | 45.55 | 37.80 | 43.12 | 52.40 | 40.27 | 33.75 | 13.77 | 3.746 |

Note:
CV % = coefficient of variation;
ND = not determined;
SD = standard deviation;
BLQ = values below the lower limit of quantification
(LLOQ: 0.200 ng/mL) were set to zero for the calculation of descriptive statistics.
[a]15 hours from post end of infusion is 24 hours from start of infusion.

TABLE 40
Summary of Pharmacokinetic Parameters of Compound A (PK set)

| | | | | | | | | | |
|---|---|---|---|---|---|---|---|---|---|
| Treatment/ | AUCinf | AUClast | Ceoi | Cmax | tmax | t½, z | CL | Vz | Vss |
| Statistics | (ng*h/mL) | (ng*h/mL) | (ng/mL) | (ng/mL) | (h) | (h) | (L/h) | (L) | (L) |

Cmpd A 44 mg (N = 25)

| | | | | | | | | | |
|---|---|---|---|---|---|---|---|---|---|
| n | 23 | 23 | 22 | 24 | 24 | 23 | 23 | 23 | 23 |
| Mean | 898.9 | 884.5 | 90.42 | 101.9 | ND | 3.794 | 51.62 | 277.7 | 128.6 |
| SD | 186.6 | 179.1 | 22.86 | 20.48 | ND | 0.7553 | 11.03 | 58.10 | 30.21 |

Cmpd A 112 mg (N = 24)

| | | | | | | | | | |
|---|---|---|---|---|---|---|---|---|---|
| n | 18 | 19 | 18 | 24 | 24 | 18 | 18 | 18 | 18 |
| Mean | 2055 | 2036 | 211.6 | 230.1 | ND | 3.927 | 57.21 | 318.7 | 139.1 |
| SD | 399.3 | 371.4 | 52.4 | 41.92 | ND | 0.8209 | 9.321 | 65.32 | 30.46 |

Note:
CV % = coefficient of variation;
ND = not determined;
SD = standard deviation.
Only Minimum, Maximum, and Median presented for tmax.

### Example III-3: MWT

On each day of dosing, four 40-minute Maintenance of Wakefulness Test (MWT) sessions were performed at 2, 4, 6, and 8 hours after the start of the infusion to evaluate the effect of Compound A on sleep latency. MWT is a validated objective measure that evaluates a person's ability to remain awake under soporific conditions.

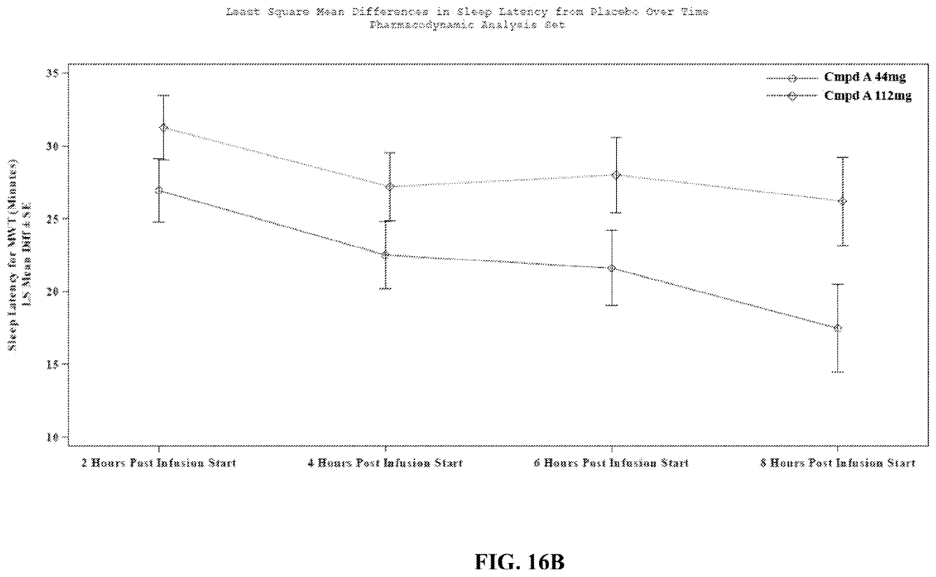

Least Square Mean (±SE) of Sleep Latency of MWT over time is shown in FIG. 16A. Least Square Mean Difference (±SE) of Sleep Latency of MWT from placebo over time is shown in FIG. 16B.

At baseline, the average sleep onset latency in the MWT was 8.2 minutes across all subjects. A large increase in mean sleep latency in the MWT was observed at 2 hours post infusion and sustained for 8 hours after start of the infusion, with mean placebo-adjusted increases of 22 minutes and 28 minutes in Compound A 44 and Compound A 112 mg mg dose groups, respectively.

### Example III-4: KSS

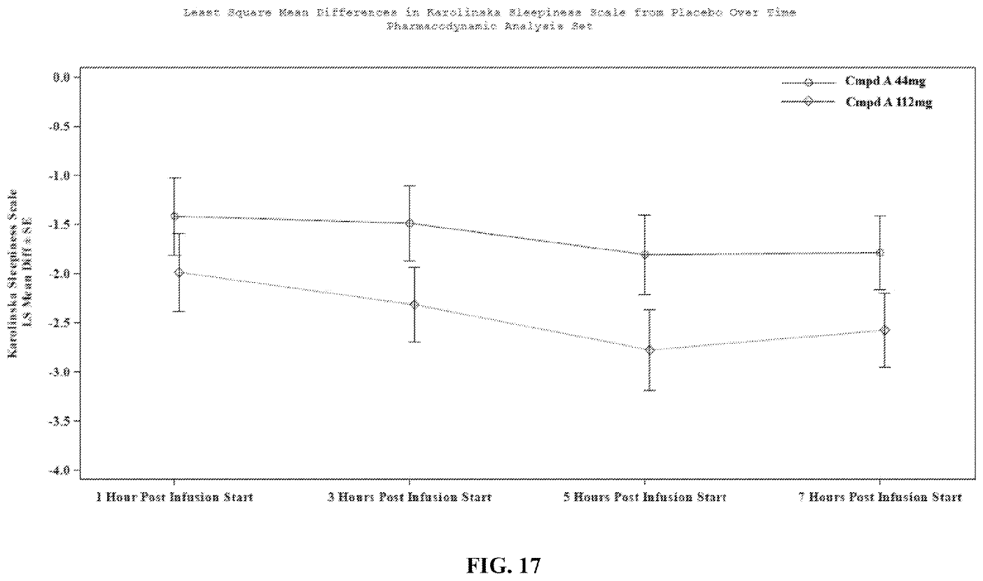

Least Square Mean Difference in KSS from placebo over time is provided FIG. 17.

KSS data supported the MWT results. At baseline, the average KSS score for all subjects was 6.3. The mean placebo-adjusted decreases in KSS were statistically significant for both Compound A 44 mg and Compound A 112 mg dose levels at −1.6 and −2.4, respectively.

### CONCLUSIONS

Single doses of Compound A 44 mg and 112 mg administered as an infusion over 9 hours were safe and well tolerated, and demonstrated remarkable positive effects on objective and subjective measures of daytime wakefulness in patients with OSA who were experiencing residual excessive daytime sleepiness despite adequate use of CPAP.

What is claimed is:

1. A method for decreasing or treating excessive sleepiness in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof,
wherein plasma concentration for Compound (I) is about 50.90 ng/mL or more for about 1 hour or more.

2. The method of claim 1, wherein orexin level in the subject is not compromised or partially compromised.

3. The method of claim 1, wherein the subject suffers from the diseases or disorders or symptoms associated with excessive sleepiness.

4. The method of claim 1, wherein the subject is sleep-deprived subject, subject with excessive sleepiness, subject with disruptive regular sleep cycle, or subject with a need to decrease sleepiness.

5. The method of claim 1, wherein the excessive sleepiness is caused by narcolepsy type 2 or idiopathic hypersomnia.

6. The method of claim 1, wherein the excessive sleepiness is caused by narcolepsy type 2.

7. The method of claim 1, wherein the excessive sleepiness is excessive daytime sleepiness.

8. The method of claim 7, wherein the excessive daytime sleepiness is caused by obstructive sleep apnea despite use of continuous positive airway pressure (CPAP).

9. The method of claim 1, wherein plasma concentration for Compound (I) is about 60.54 ng/mL or more for about 1 hour or more.

10. The method of claim 1, wherein plasma concentration for Compound (I) is about 60.54 ng/mL or more for about 4 hours or more.

11. The method of claim 1, wherein plasma concentration for Compound (I) is about 150 ng/mL or more for about 4 hours or more.

12. The method of claim 1, wherein further plasma concentration for Compound (I) is about a half of Cmax for administration of Compound (I) or less at about 1 hour prior to sleep time.

13. The method of claim 1, wherein further plasma concentration for Compound (I) is about a quarter of Cmax for administration of Compound (I) or less at about 1 hour prior to sleep time.

14. The method of claim 1, wherein further plasma concentration for Compound (I) is about a half of 50.90 ng/mL or less at about 1 hour prior to sleep time.

15. The method of claim 1, wherein further plasma concentration for Compound (I) is about a quarter of 50.90 ng/mL or less at about 1 hour prior to sleep time.

16. The method of claim 1, wherein Cmax for administration of Compound (I) is about 94.66 ng/mL or more.

17. The method of claim 1, wherein AUC∞ for administration of Compound (I) is about 829 ng*h/mL or more.

18. The method of claim 1, wherein the excessive sleepiness is excessive daytime sleepiness or excessive sleepiness during working hours.

19. The method of claim 1, wherein the administration is non-oral administration.

20. The method of claim 19, wherein the non-oral administration is intravenous administration, subcutaneous administration, transdermal administration, intradermal administration or transmucosal administration.

21. The method of claim 1, wherein the administration is a single daily administration or a multiple daily administration.

22. A method for treating narcolepsy type 2 or idiopathic hypersomnia in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof,
wherein plasma concentration for Compound (I) is about 50.90 ng/mL or more for about 1 hour or more.

23. A method for treating shift work disorder, shift work sleep disorder or jet lag syndrome in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof,
wherein plasma concentration for Compound (I) is about 50.90 ng/mL or more for about 1 hour or more.

24. The method of claim 22 or claim 23, wherein Cmax for administration of Compound (I) is about 94.66 ng/mL or more.

25. The method of claim 22 or claim 23, wherein AUC∞ for administration of Compound (I) is about 829 ng*h/mL or more.

26. The method of claim 22 or claim 23, wherein the administration is non-oral administration.

27. The method of claim 26, wherein the non-oral administration is intravenous administration, subcutaneous administration, transdermal administration, intradermal administration or transmucosal administration.

28. The method of claim 22 or claim 23, wherein the administration is a single daily administration or a multiple daily administration.

29. A method for increasing wakefulness in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof,
wherein plasma concentration for Compound (I) is about 50.90 ng/mL or more for about 1 hour or more.

30. The method of claim 29, wherein orexin level in the subject is not compromised or partially compromised.

31. The method of claim 29, wherein the subject is sleep-deprived subject, subject with excessive sleepiness, subject with disruptive regular sleep cycle or subject with a need to decrease sleepiness.

32. The method of claim 29, wherein the administration is non-oral administration.

33. A method for increasing sleep latency in maintenance of wakefulness test (MWT) in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof,
wherein plasma concentration for Compound (I) is about 50.90 ng/mL or more for about 1 hour or more.

34. The method of claim 33, wherein orexin level in the subject is not compromised or partially compromised.

35. The method of claim 33, wherein the subject is sleep-deprived subject, subject with excessive sleepiness, subject with disruptive regular sleep cycle or subject with a need to decrease sleepiness.

36. The method of claim 33, wherein the administration is non-oral administration.

37. A method for decreasing or improving objective sleepiness or sleepiness measured by EEG in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof,
wherein plasma concentration for Compound (I) is about 50.90 ng/mL or more for about 1 hour or more.

38. The method of claim 37, wherein orexin level in the subject is not compromised or partially compromised.

39. A method for improving Karolinska Sleepiness Scale (KSS) rating in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof,
wherein plasma concentration for Compound (I) is about 50.90 ng/mL or more for about 1 hour or more.

40. The method of claim 39, wherein orexin level in the subject is not compromised or partially compromised.

41. The method of claim 39, wherein the subject is sleep-deprived subject, subject with excessive sleepiness, subject with disruptive regular sleep cycle or subject with a need to decrease sleepiness.

42. The method of claim 39, wherein the administration is non-oral administration.

43. A method for decreasing or improving subjective sleepiness in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein plasma concentration for Compound (I) is about 50.90 ng/mL or more for about 1 hour or more.

44. The method of claim 43, wherein orexin level in the subject is not compromised or partially compromised.

45. A method for increasing wakefulness or decreasing excessive sleepiness for about 4 hours or more in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof,
wherein orexin level in the subject is not compromised or partially compromised; and plasma concentration for Compound (I) is maintained at about 50.90 ng/mL or more.

46. The method of claim 45, wherein the subject is sleep-deprived subject, subject with excessive sleepiness, subject with disruptive regular sleep cycle or subject with a need to decrease sleepiness.

47. The method of claim 45, which is the method for increasing wakefulness or decreasing excessive sleepiness for about 6 hours or more.

48. The method of claim 45, which is the method for increasing wakefulness or decreasing excessive sleepiness for about 8 hours or more.

49. The method of claim 45, wherein plasma concentration for Compound (I) is maintained at about 150 ng/mL or more.

50. The method of claim 45, wherein further plasma concentration for Compound (I) is about a half of Cmax for administration of Compound (I) or less at about 1 hour prior to sleep time.

51. The method of claim 45, wherein further plasma concentration for Compound (I) is about a quarter of Cmax for administration of Compound (I) or less at about 1 hour prior to sleep time.

52. The method of claim 45, wherein further plasma concentration for Compound (I) is about a half of 50.90 ng/mL or less at about one hour prior to sleep time.

53. The method of claim 45, wherein further plasma concentration for Compound (I) is about a quarter of 50.90 ng/mL or less at about one hour prior to sleep time.

54. The method of any of claims 1-53, wherein the effective amount is between about 20 mg to about 500 mg.

55. The method of claim 54, wherein the effective amount is between about 30 mg to about 300 mg.

56. The method of claim 54, wherein the effective amount is between about 40 mg to about 300 mg.

57. The method of claim 54, wherein the effective amount is between about 40 mg to about 200 mg.

58. The method of claim 54, wherein the effective amount is gradually increased within the range from about 20 mg to about 500 mg.

59. The method of claim 54, wherein the effective amount is gradually increased within the range from about 40 mg to about 200 mg.

60. The method of any of claims 1-53, wherein Compound (I) is administered at least once per day.

61. The method of any of claims 1-53, further comprising administering one or more additional therapies.

62. The method of claim 61, wherein the one or more additional therapies is selected from a stimulant, antidepressant, central nervous system depressant, and histamine 3 (H3) receptor antagonist.

63. A method for improving Epworth Sleepiness Scale (ESS) rating in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein the plasma concentration for Compound (I) is about 38.21 ng/mL or more for about 1 hour or more.

64. A method for treating narcolepsy type 2 in a subject in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein, the plasma concentration for Compound (I) is about 38.21 ng/mL or more for about 1 hour or more.

65. A method for decreasing or treating excessive daytime sleepiness in a subject with obstructive sleep apnea who uses continuous positive airway pressure (CPAP) in need thereof, comprising administering to the subject an effective amount of methyl 3-((methylsulfonyl)amino)-2-(((4-phenylcyclohexyl)oxy)methyl)piperidine-1-carboxylate (Compound (I)), or a salt thereof, wherein the plasma concentration for Compound (I) is about 42.08 ng/mL or more for about 1 hour or more.

66. The method of any of claims 1-65, wherein Compound (I) is optical active compound.

67. The method of any of claims 1-65, wherein Compound (I) is methyl (2R,3S)-3-((methylsulfonyl)amino)-2-(((cis-4-phenylcyclohexyl)oxy)methyl)piperidine-1-carboxylate (Compound A).

68. A pharmaceutical composition comprising (a) methyl 3-((methylsulfonyl)amino)-2-(((4-phenylcyclohexyl)oxy)methyl)piperidine-1-carboxylate (Compound (I)), or a salt thereof, and (b) a pharmaceutically acceptable carrier therefor, which provides a plasma concentration for Compound (I) of about 50.90 ng/mL or more for about 1 hour or more.

69. The pharmaceutical composition of claim 68, which provides a Cmax for Compound (I) of about 94.66 ng/mL or more.

70. The pharmaceutical composition of claim 68, which provides an AUC∞ for Compound (I) of about 829 ng*h/mL or more.

71. The pharmaceutical composition of claim 68, which is formulated for non-oral administration.

72. The pharmaceutical composition of any of claims 68-71, wherein Compound (I) is optical active compound.

73. The pharmaceutical composition of any of claims 68-71, wherein Compound (I) is methyl (2R,3S)-3-((methylsulfonyl)amino)-2-(((cis-4-phenylcyclohexyl)oxy)methyl)piperidine-1-carboxylate (Compound A).
